(19) United States
(12) Patent Application Publication
(10) Pub. No.: US 2010/0022527 A1
(43) Pub. Date: Jan. 28, 2010

(54) BIPHENYL SUBSTITUTED SPIROTETRONIC ACIDS AND THEIR USE FOR THE TREATMENT OF RETROVIRAL DISEASES

(75) Inventors: Dirk HEIMBACH, Duesseldorf (DE); Adrian TERSTEEGEN, Wuppertal (DE); Kai THEDE, Berlin (DE); Reinhold WELKER, Bensheim (DE); Beate FAST, Ludwigshafen (DE); Arnold PAESSENS, Haan (DE); Frank DITTMER, Duesseldorf (DE); Rudolph SCHOHE-LOOP, Wuppertal (DE); Axel HARRENGA, Wuppertal (DE); Alexander HILLISCH, Velbert (DE); Kerstin HENNINGER, Wuppertal (DE); Walter HUEBSCH, Wuppertal (DE); Marcus BAUSER, Wuppertal (DE); Daniela PAULSEN, Wuppertal (DE); Alexander BIRKMANN, Wuppertal (DE); Thomas BRETSCHNEIDER, Lohmar (DE); Reiner FISCHER, Monheim (DE); Susanne GRESCHAT, Duesseldorf (DE); Andreas URBAN, Hattingen (DE); Steffen WILDUM, Schwelm (DE)

Correspondence Address: MORRISON & FOERSTER LLP, 12531 HIGH BLUFF DRIVE, SUITE 100, SAN DIEGO, CA, 92130-2040, US

(73) Assignee: AiCuris GMBH & Co. KG, Wuppertal (DE)

(21) Appl. No.: 12/392,844

(22) Filed: Feb. 25, 2009

Related U.S. Application Data
(63) Continuation of application No. PCT/EP2007/007130, filed on Aug. 13, 2007.

(30) Foreign Application Priority Data
Aug. 25, 2006 (DE) 10 2006 039 912.9

(51) Int. Cl. A61K 31/5377; C07D 491/10; A61K 31/438; C07D 413/12; C07D 307/94; A61K 31/34; A61P 31/12; A61P 31/18

(52) U.S. Cl. 514/231.5; 546/16; 514/278; 544/70; 549/265; 514/462

(57) ABSTRACT

The present invention relates to novel substituted spirotetronic acids (I) in which R¹ and R², together with the carbon atom to which they are bonded, form a group of the formula (1), (2), (3) or (4), whereby * represents the carbon atom to which R¹ and R² are bonded, to methods for their preparation, their use for the treatment and/or prophylaxis of diseases, as well as their use for the manufacture of medicaments for the treatment and/or prophylaxis of diseases, in particular of retroviral disorders, in people and/or animals.

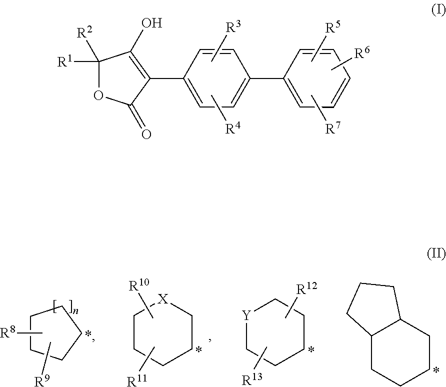

### CROSS-REFERENCE TO RELATED APPLICATIONS

This application is a continuation of co-pending international patent application PCT/EP2007/007130 filed on Aug. 13, 2007, and claims priority of German patent application 10 2006 039 912.9 filed on Aug. 25, 2006. The contents of the above-referenced applications are incorporated herein by this reference in their entireties.

### BACKGROUND OF THE INVENTION

### Field of the Invention

The present invention relates to novel substituted spirotetronic acids, methods for their preparation, their use for the treatment and/or prophylaxis of diseases as well as their use for the manufacture of medicaments for the treatment and/or prophylaxis of diseases, in particular retroviral diseases, in humans and/or animals.

HIV (virus of human immune deficiency) causes a chronically persistent progressive infection. The disease runs through different stages from the asymptomatic infection up to the clinical picture AIDS (Acquired Immunodeficiency Syndrome). AIDS is the final stage of the disease caused by infection. Characteristic of the HIV AIDS disease is the long clinical latency period with persistent viremia which in the final stage leads to the failure of the immune defense.

Through the introduction of anti-HIV combination therapy in the 1990s it was possible to sustainably slow the progression of the disease and thus to substantially increase the life expectance of HIV-infected patients (Palella, et al., N. Engl. J. Med. 1998, 238, 853-860).

The anti-HIV substances currently on the market inhibit the replication of the HI virus by inhibition of the essential viral enzymes reverse transcriptase (RT), the protease or the HIV fusion (review in Richman, Nature 2001, 410, 995-1001). There are two classes of RT inhibitors: nucleosidic RT inhibitors (NRTI) act through competitive inhibition or chain termination during DNA polymerization. Non-nucleosidic RT inhibitors (NNRTI) bind allosterically to a hydrophobic pocket in the vicinity of the active center of the RT and mediate a conformational change in the enzyme. The currently available protease inhibitors (PI) on the other hand block the active center of the viral protease and thus prevent the maturation of newly formed particles to infections virions.

Since monotherapy with the currently available anti-HIV medicaments leads within a very short time to therapy failure through the selection of resistant viruses, normally a combination therapy with several anti-HIV substances from different classes is undertaken (highly active antiretroviral therapy=HAART; Carpenter, et al., J. Am. Med. Assoc. 2000, 283, 381-390).

In spite of the advances in anti-retroviral chemotherapy more recent studies show that an eradication of HIV and an associated cure of the HIV infection is not to be expected with the available medicaments: latent virus remains in dormant lymphocytes and represents a reservoir for a reactivation and thus for a renewed virus proliferation (Finzi, et al., Nature Med. 1999, 5, 512-517; Ramratnam, et al., Nature Med. 2000, 6, 82-85). HIV-infected patients are thus dependent on an efficient antiviral therapy throughout their lifetime. In spite of combination therapy a selection of resistant viruses occurs after a certain time. Since characteristic resistance mutations accumulate for every therapeutic class the failure of one therapy often means a loss of efficacy of the complete substance class.

The occurrence of resistance is usually favored by the poor compliance of the patient, which is brought about by an unfavorable side effect profile and complicated dosing regime of the anti-HIV medicaments.

Thus there is urgent need for new therapeutic options for combating HIV infections. For this the identification of new chemical lead structures is important and a pressing objective of HIV therapy research, which address either a new target in the replication of HIV and/or are active against the growing number of resistant clinical HIV isolates.

WO 99/55673, DE 4014420 and WO 2006/000355 describe i.a. spirotetronic acids as pesticides and herbicides. WO 96/29333 and WO 95/07901 describe tetronic acids for the treatment of HIV.

The invention relates to compounds of formula

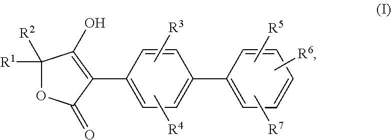

in which

R¹ and R² together with the carbon atom to which they are bonded form a group of formula

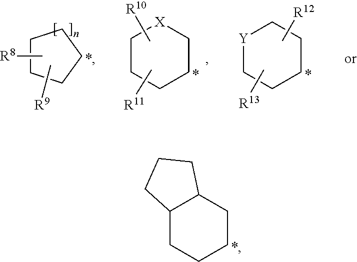

whereby

represents the carbon atom to which R¹ and R² are bonded,

n represents the number 1, 2 or 3,

X represents an oxygen atom, a sulfur atom or NR¹⁴,
whereby
R¹⁴ represents C₁-C₆-alkyl, C₂-C₄-alkenyl, C₁-C₄-alkylsulfonyl, benzylsulfonyl, —(CH₂)ₒCOR¹⁶, —(CH₂)ₚCONR¹⁷R¹⁸, (CH₂)_{q}NR²⁴COR²⁵ or —(CH₂)ᵥNR²⁶SO₂R²⁷,
   whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²²,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
      wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
      and
      R²² represents C₃-C₇-cycloalkyl, phenyl, 5- to 10-membered heterocyclyl or 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   and
   whereby
   o represents a number 0, 1, 2 or 3,
   p represents a number 0, 1, 2 or 3,
   q represents a number 2 or 3,
   v represents a number 2 or 3,
   R¹⁶ represents C₁-C₆-alkyl, C₂-C₄-alkenyl, C₁-C₆-alkoxy, phenyl, benzyloxy or 5- to 10-membered heterocyclyl,
      whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   R¹⁷ represents hydrogen, C₁-C₄-alkyl or phenyl, whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
      wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   R¹⁸ represents hydrogen or C₁-C₄-alkyl,
   R²⁴ represents hydrogen or C₁-C₄-alkyl,
   R²⁵ represents C₁-C₆-alkyl, C₂-C₄-alkenyl, C₁-C₆-alkoxy, phenyl, benzyloxy or 5- to 10-membered heterocyclyl,
      whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   R²⁶ represents hydrogen or C₁-C₄-alkyl,
   R²⁷ represents C₁-C₆-alkyl, C₂-C₄-alkenyl, phenyl or 5- to 10-membered heterocyclyl,
      whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,

Y represents an oxygen atom, a sulfur atom or NR¹⁵,
whereby
R¹⁵ represents C₁-C₆-alkyl, C₂-C₄-alkenyl, C₁-C₄-alkylsulfonyl, benzylsulfonyl, —(CH₂)ᵣCOR¹⁹, —(CH₂)ₛCONR²⁰R²¹, —(CH₂)ₜNR²⁸COR²⁹ or —(CH₂)_{w}NR³⁰SO₂R³¹,
   whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²³,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
      wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   and
   R²³ represents C₃-C₇-cycloalkyl, phenyl, 5- to 10-membered heterocyclyl or 5- to 10-membered heteroaryl, wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
r represents a number 0, 1, 2 or 3,
s represents a number 0, 1, 2 or 3,
t represents a number 2 or 3,
w represents a number 2 or 3,
R¹⁹ represents C₁-C₆-alkyl, C₂-C₄-alkenyl, C₁-C₆-alkoxy, phenyl, benzyloxy or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²⁰ represents hydrogen, C₁-C₄-alkyl or phenyl,
   whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
      wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²¹ represents hydrogen or C₁-C₄-alkyl,
R²⁸ represents hydrogen or C₁-C₄-alkyl,
R²⁹ represents C₁-C₆-alkyl, C₂-C₄-alkenyl, C₁-C₆-alkoxy, phenyl, benzyloxy or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R³⁰ represents hydrogen or C₁-C₄-alkyl,
R³¹ represents C₁-C₆-alkyl, C₂-C₄-alkenyl, phenyl or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R⁸ represents hydrogen, oxo, trifluoromethyl, trifluoromethoxy, C₁-C₄-alkyl, C₁-C₄-alkoxy or C₁-C₄-alkylthio,
R⁹ represents hydrogen, C₁-C₄-alkyl or C₁-C₄-alkoxy,
R¹⁰ represents hydrogen or C₁-C₄-alkyl,
R¹¹ represents hydrogen or C₁-C₄-alkyl,
R¹² represents hydrogen or C₁-C₄-alkyl,
R¹³ represents hydrogen or C₁-C₄-alkyl,

R³ represents hydrogen, halogen, cyano, methyl, ethyl, methoxy, ethoxy or phenoxy,

R⁴ represents hydrogen, halogen, methyl, ethyl, methoxy or ethoxy,

R⁵ represents hydrogen, halogen, cyano, nitro, hydroxy, amino, trifluoromethyl, trifluoromethoxy, hydroxycarbonyl, aminocarbonyl, hydroxymethyl, aminomethyl, C₁-C₄-alkyl, C₁-C₄-alkoxy, C₁-C₄-alkylamino, C₁-C₄-alkylthio, C₁-C₄-alkylcarbonyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonyl, C₁-C₄-alkylsulfonylamino, C₂-C₄-alkenylsulfonylamino, C₁-C₄-alkylsulfonyl(C₁-C₄-alkyl)amino, benzylsulfonylamino, 5- or 6-membered heteroarylsulfonylamino or 5- to 7-membered heterocyclyl,

whereby alkylaminocarbonyl, alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino,

R⁶ represents hydrogen, halogen, C₁-C₄-alkyl or C₁-C₄-alkoxy,

R⁷ represents hydrogen, halogen, C₁-C₄-alkyl or C₁-C₄-alkoxy,

or

R⁵ and R⁶ are bonded to neighboring carbon atoms and together with the carbon atoms to which they are bonded form a 1,3-dioxolane,

and their salts, their solvates and the solvates of their salts,

for the treatment and/or prophylaxis of diseases.

Compounds of the invention are the compounds of formula (I) and their salts, solvates and solvates of the salts; the compounds encompassed by formula (I) of the formulae named in the following and their salts, solvates and solvates of their salts as well as the compounds encompassed by formula (I) named in the following as exemplary embodiments and their salts, solvates and solvates of the salts insofar as the compounds encompassed by formula (I) named in the following are not already salts, solvates and solvates of the salts.

The compounds of the invention can depending on their structure exist in stereoisomeric forms (enantiomer, diastereomers). The invention therefore comprises the enantiomers or diastereomers and their respective mixtures. The stereoisomerically uniform components can be isolated from such mixtures of enantiomers and/or diastereomers by known methods.

Where the compounds of the invention can exist in tautomeric forms the present invention encompasses all tautomeric forms.

Salts preferred for the purpose of the present invention are physiologically acceptable salts of the compounds of the invention. However, also included are salts which themselves are not suitable for pharmaceutical applications but can be used, for example, for the isolation or purification of the compounds of the invention.

Physiologically acceptable salts of the compounds of the invention include acid addition salts of mineral acids, carboxylic acids and sulfonic acids, e.g., salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartatic acid, malic acid, citric acid, fumaric acid, maleic acid and benzoic acid.

Physiologically acceptable salts of the compounds of the invention also include salts of common bases, such as, by way of example and preferably, alkali metal salts (e.g., sodium and potassium salts), alkaline earth metal salts (e.g., calcium and magnesium salts) and ammonium salts, derived from ammonia or organic amines with 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates for the purpose of the invention refer to these forms of the compounds of the invention which in the solid or liquid state form a complex through coordination with solvent molecules. Hydrates are a special form of the solvates in which coordination takes place with water.

For the purpose of the present invention the substituents have the following meaning unless otherwise specified.

Alkyl as well the alkyl parts in alkoxy, alkylamino, alkylthio, alkylcarbonyl, alkylsulfonyl, alkoxycarbonyl, alkylaminocarbonyl, alkylaminosulfonyl, alkylcarbonylamino, alkoxycarbonylamino, alkylsulfonylamino and alkylsulfonyl(C₁-C₄-alkyl)amino represent linear or branched alkyl and unless otherwise stated comprise C₁-C₆-alkyl, in particular C₁-C₄-alkyl, such as for example methyl, ethyl, propyl, isopropyl, butyl, isobutyl.

Alkenyl represents a straight-chain or branched alkenyl radical having 2 to 4 carbon atoms. Preferred is a straight-chain alkenyl radical having 2 to 3 carbon atoms. Named by way of example and preferably are: vinyl, allyl, n-prop-1-en-1-yl and n-but-2-en-1-yl.

For the purpose of the invention alkoxy preferably represents a straight-chain or branched alkoxy radical in particular having 1 to 6, 1 to 4 or 1 to 3 carbon atoms. Preferred is a straight-chain or branched alkoxy radical having 1 to 3 carbon atoms. Named by way of example and preferably are: methoxy, ethoxy, n-propoxy, isopropoxy, t-butoxy, n-pentoxy and n-hexoxy.

For the purpose of the invention alkylamino represents an amino group having one or two straight-chain or branched alkyl substituents (selected independently of one another) preferably having 1 to 6, 1 to 4 or 1 to 2 carbon atoms. By way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino. C₁-C₃-Alkylamino represents for example a monoalkylamino radical having 1 to 3 carbon atoms or a dialkylamino radical having 1 to 3 carbon atoms each per alkyl substituent.

Alkylthio by way of example and preferably represents methylthio, ethylthio, n-propylthio, isopropylthio, tert.-butylthio, n-pentylthio and n-hexylthio.

Alkylcarbonyl by way of example and preferably represents methylcarbonyl, ethylcarbonyl, n-propylcarbonyl, iso-propylcarbonyl, n-butylcarbonyl and tert-butylcarbonyl.

Alkylsulfonyl by way of example and preferably represents methylsulfonyl, ethylsulfonyl, n-propylsulfonyl, isopropylsulfonyl, tert.-butylsulfonyl, n-pentylsulfonyl and n-hexylsulfonyl.

Alkoxycarbonyl by way of example and preferably represents methoxycarbonyl, ethoxycarbonyl, n-propoxycarbonyl, isopropoxycarbonyl, t-butoxycarbonyl, n-pentoxycarbonyl and n-hexoxycarbonyl.

For the purpose of the invention alkylaminocarbonyl represents an aminocarbonyl group having one or two straight-chain or branched alkyl substituents (selected independently of one another) preferably having 1 to 6, 1 to 4 or 1 to 2 carbon atoms. By way of example and preferably methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-tert-butyl-N-methylaminocarbonyl, N-ethyl-N-n-pentylaminocarbonyl and N-n-hexyl-N-methylaminocarbonyl. C₁-C₃-Alkylaminocarbonyl by way of example represents a monoalkylaminocarbonyl radical having 1 to 3 carbon atoms or a dialkylaminocarbonyl radical having 1 to 3 carbon atoms each per alkyl substituent.

For the purpose of the invention alkylaminosulfonyl represents an aminosulfonyl group having one or two straight-chain or branched alkyl substituents (selected independently of one another) preferably having 1 to 6, 1 to 4 or 1 to 2 carbon atoms. By way of example and preferably methylaminosulfonyl, ethylaminosulfonyl, n-propylaminosulfonyl, isopropylaminosulfonyl, tert-butylaminosulfonyl, n-pentyl-aminosulfonyl, n-hexylaminosulfonyl, N,N-dimethylaminosulfonyl, N,N-diethylaminosulfonyl, N-ethyl-N-methylaminosulfonyl, N-methyl-N-n-propylaminosulfonyl, N-isopropyl-N-n-propylaminosulfonyl, N-tert-butyl-N-methylaminosulfonyl, N-ethyl-N-n-pentylaminosulfonyl and N-n-hexyl-N-methylaminosulfonyl. C₁-C₃-Alkylaminosulfonyl, by way of example, represents a monoalkylaminosulfonyl radical having 1 to 3 carbon atoms or a dialkylaminosulfonyl radical having 1 to 3 carbon atoms each per alkyl substituent.

Alkylcarbonylamino by way of example and preferably represents methylcarbonylamino, ethylcarbonylamino, n-propylcarbonylamino, isopropylcarbonylamino, n-butylcarbonylamino and tert-butylcarbonylamino.

Alkoxycarbonylamino by way of example and preferably represents methoxycarbonylamino, ethoxycarbonylamino, n-propoxycarbonylamino, isopropoxycarbonylamino, t-butoxycarbonylamino, n-pentoxycarbonylamino and n-hexoxycarbonylamino.

Alkylsulfonylamino by way of example and preferably represents methylsulfonylamino, ethylsulfonylamino, n-propylsulfonylamino, isopropylsulfonylamino, tert.-butylsulfonylamino, n-pentylsulfonylamino and n-hexylsulfonylamino.

Alkenylsulfonylamino by way of example and preferably represents vinylsulfonylamino, allylsulfonylamino, n-prop-1-en-1-ylsulfonylamino and n-but-2-en-1-ylsulfonylamino.

Cycloalkyl represents a cycloalkyl group usually having 3 to 7 carbon atoms, by way of example and preferably cyclopropyl, cyclobutyl, cyclopentyl, cyclohexyl and cycloheptyl.

Cycloalkylaminocarbonyl by way of example and preferably represents cyclopropylaminocarbonyl, cyclobutylaminocarbonyl, cyclopentylaminocarbonyl, cyclohexylaminocarbonyl and cycloheptylaminocarbonyl.

Heterocyclyl represents a mono or bicyclic heterocyclic radical usually having 3 to 10, preferably 5 to 8 ring atoms and up to 3, preferably up to 2 heteroatoms and/or hetero groups from the series N, O, S, SO, SO₂, whereby a nitrogen atom can also form an N-oxide. The heterocyclyl radicals can be saturated or partially unsaturated. Preferred are 5- to 8-membered, monocyclic saturated heterocyclyl radicals having up to two heteroatoms from the series O, N and S, by way of example and preferably oxetan-3-yl, pyrrolidin-2-yl, pyrrolidin-3-yl, pyrrolinyl, tetrahydrofuranyl, tetrahydrothienyl, pyranyl, piperidin-1-yl, piperidin-2-yl, piperidin-3-yl, piperidin-4-yl, thiopyranyl, morpholin-1-yl, morpholin-2-yl, morpholin-3-yl, perhydroazepinyl, piperazin-1-yl, piperazin-2-yl.

Heteroaryl represents a 5- to 10-membered aromatic mono- or bicyclic heterocycle, preferably a 5- or 6-membered aromatic monocyclic heterocycle having up to 3 heteroatoms from the series S, O and/or N, whereby the heterocycle can also exist in the form of the N-oxide, for example indolyl, 1H-indazolyl, 1H-1,2,3-benzotriazolyl, 1H-benzimidazolyl, pyridyl, pyrimidyl, thienyl, furyl, pyrrolyl, thiazolyl, pyrazolyl, thiadiazolyl, N-triazolyl, isoxazolyl, oxazolyl or imidazolyl. Preferred are pyridyl, thienyl, furyl and thiazolyl.

Halogen represents fluorine, chlorine, bromine or iodine, whereby fluorine and chlorine are preferred unless otherwise stated.

The radical definitions given above generally or in preferred ranges apply both for the final products of formula (I) and in each case for the corresponding starting materials and intermediates required for the preparation.

The radical definitions stated individually in the respective combinations and preferred combinations of radicals are also arbitrarily replaced by radical definitions of other combinations independently of the respectively stated combinations of radicals.

The invention also relates to compounds of formula (I) in which R¹ and R² together with the carbon atom to which they are bonded form a group of formula

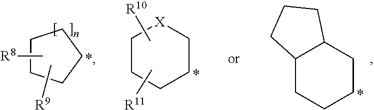

whereby

* represents the carbon atom to which R¹ and R² are bonded,

n represents the number 2,

X represents an oxygen atom, a sulfur atom or NR¹⁴,
whereby
R¹⁴ represents C₁-C₆-alkyl, C₂-C₄-alkenyl, C₁-C₄-alkylsulfonyl, benzylsulfonyl, —(CH₂)ₒCOR¹⁶ or —(CH₂)ₚCONR¹⁷R¹⁸,
   whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₃-C₇-cycloalkyl, phenyl, 5- to 10-membered heterocyclyl and 5- to 10-membered heteroaryl,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   and
   whereby
   o represents a number 0, 1, 2 or 3,
   p represents a number 0, 1, 2 or 3,
   R¹⁶ represents C₁-C₆-alkyl, C₂-C₄-alkenyl, C₁-C₆-alkoxy, phenyl, benzyloxy or 5- to 10-membered heterocyclyl,
      whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   R¹⁷ represents hydrogen, C₁-C₄-alkyl or phenyl,
      whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
         wherein phenyl and heteroaryl for their part can be substituted with 1 to 3 substituents, whereby the substituents are selected independently of one another from the group consisting of halogen, cyano, trifluoromethyl, trifluoromethoxy and C₁-C₄-alkyl,
   R¹⁸ represents hydrogen or C₁-C₄-alkyl,
R⁸ represents hydrogen, oxo, trifluoromethyl, trifluoromethoxy, C₁-C₄-alkyl, C₁-C₄-alkoxy or C₁-C₄-alkylthio,
R⁹ represents hydrogen, C₁-C₄-alkyl or C₁-C₄-alkoxy,
R¹⁰ represents hydrogen,
R¹¹ represents hydrogen,

R³ represents hydrogen, halogen, methyl, ethyl, methoxy, ethoxy or phenoxy,

R⁴ represents hydrogen, halogen, methyl, ethyl, methoxy or ethoxy,

R⁵ represents hydrogen, halogen, cyano, nitro, hydroxy, amino, trifluoromethyl, trifluoromethoxy, hydroxycarbonyl, aminocarbonyl, hydroxymethyl, aminomethyl, C₁-C₄-alkyl, C₁-C₄-alkoxy, C₁-C₄-alkylamino, C₁-C₄-alkylthio, C₁-C₄-alkylcarbonyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonyl, C₁-C₄-alkylsulfonylamino, C₂-C₄-alkenylsulfonylamino, C₁-C₄-alkylsulfonyl(C₁-C₄-alkyl)amino, benzylsulfonylamino, 5- or 6-membered heteroarylsulfonylamino or 5- to 7-membered heterocyclyl,
whereby alkylaminocarbonyl, alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino,

R⁶ represents hydrogen, halogen, C₁-C₄-alkyl or C₁-C₄-alkoxy,

R⁷ represents hydrogen,

or

R⁵ and R⁶ are bonded to neighboring carbon atoms and together with the carbon atoms to which they are bonded form a 1,3-dioxolane,

and their salts, their solvates and the solvates of their salts,

for the treatment and/or prophylaxis of diseases.

The invention also relates to compounds of formula (I) in which

R¹ and R² together with the carbon atom to which they are bonded form a group of formula

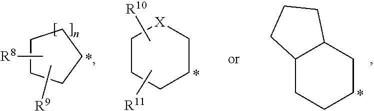

whereby
* represents the carbon atom to which R¹ and R² are bonded,
n represents the number 2,
X represents NR¹⁴,
   whereby
R¹⁴ represents C₁-C₄-alkyl, C₂-C₄-alkenyl, benzylsulfonyl, —(CH₂)ₒCOR¹⁶ or —(CH₂)ₚCONR¹⁷R¹⁸,
   whereby alkyl and alkenyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₃-C₇-cycloalkyl, phenyl, 5- to 10-membered heterocyclyl and 5- to 10-membered heteroaryl,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   and
   whereby
   o represents a number 1 or 2,
   p represents a number 1 or 2,
   R¹⁶ represents C₁-C₄-alkyl, C₁-C₄-alkoxy, phenyl or benzyloxy,
   R¹⁷ represents hydrogen, C₁-C₄-alkyl or phenyl,
      whereby alkyl can be substituted with a substituent, whereby the substituent is selected from the group consisting of methoxy, phenyl and 5- or 6-membered heteroaryl,
         wherein phenyl for its part can be substituted with 1 to 3 substituents, whereby the substituents are selected independently of one another from the group consisting of halogen, cyano, trifluoromethyl, trifluoromethoxy and C₁-C₄-alkyl,
   R¹⁸ represents hydrogen,
R⁸ represents hydrogen, C₁-C₄-alkyl or C₁-C₄-alkoxy,
R⁹ represents hydrogen or C₁-C₄-alkyl,
R¹⁰ represents hydrogen,
R¹¹ represents hydrogen,

R³ represents hydrogen, halogen, methyl, ethoxy or phenoxy,

R⁴ represents hydrogen, halogen or methyl,

R⁵ represents hydrogen, halogen, cyano, hydroxy, trifluoromethyl, trifluoromethoxy, hydroxycarbonyl, aminocarbonyl, hydroxymethyl, C₁-C₄-alkyl, C₁-C₄-alkoxy, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonyl, C₁-C₄-alkylsulfonylamino, C₂-C₄-alkenylsulfonylamino, C₁-C₄-alkylsulfonyl(C₁-C₄-alkyl)amino, benzylsulfonylamino or 5- or 6-membered heteroarylsulfonylamino,
whereby alkylaminocarbonyl, alkylcarbonylamino and alkylsulfonylamino can be substituted with a substituent, whereby the substituted is selected from the group consisting of amino, C₁-C₄-alkylamino, morpholinyl and pyrrolidinyl,

R⁶ represents hydrogen, halogen, C₁-C₄-alkyl or C₁-C₄-alkoxy,

R⁷ represents hydrogen,

and their salts, their solvates and the solvates of their salts,

for the treatment and/or prophylaxis of diseases.

The invention furthermore relates to compounds of formula (I), in which

R¹ and R² together with the carbon atom to which they are bonded form a group of formula

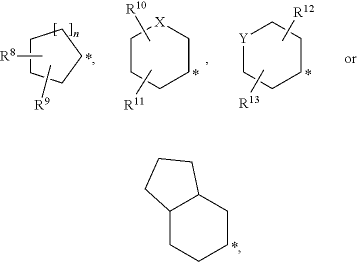

whereby
* represents the carbon atom to which R¹ and R² are bonded,
n represents the number 1, 2 or 3,
X represents an oxygen atom, a sulfur atom or NR¹⁴,
   whereby
R¹⁴ represents C₁-C₆-alkyl, C₂-C₄-alkenyl, C₁-C₄-alkylsulfonyl, benzylsulfonyl, —(CH₂)ₒCOR¹⁶, —(CH₂)ₚCONR¹⁷R¹⁸, —(CH₂)_{q}NR²⁴COR²⁵ or —(CH₂)ᵥNR²⁶SO₂R²⁷,
   whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²²,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
      wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   and
   R²² represents C₃-C₇-cycloalkyl, phenyl, 5- to 10-membered heterocyclyl or 5- to 10-membered heteroaryl,
      wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
o represents a number 0, 1, 2 or 3,
p represents a number 0, 1, 2 or 3,
q represents a number 2 or 3,
v represents a number 2 or 3,
R¹⁶ represents C₁-C₆-alkyl, C₂-C₄-alkenyl, C₁-C₆-alkoxy, phenyl, benzyloxy or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R¹⁷ represents hydrogen, C₁-C₄-alkyl or phenyl,
   whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
      wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R¹⁸ represents hydrogen or C₁-C₄-alkyl,
R²⁴ represents hydrogen or C₁-C₄-alkyl,
R²⁵ represents C₁-C₆-alkyl, C₂-C₄-alkenyl, C₁-C₆-alkoxy, phenyl, benzyloxy or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²⁶ represents hydrogen or C₁-C₄-alkyl,
R²⁷ represents C₁-C₆-alkyl, C₂-C₄-alkenyl, phenyl or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,

Y represents an oxygen atom, a sulfur atom or NR¹⁵,
whereby
R¹⁵ represents C₁-C₆-alkyl, C₂-C₄-alkenyl, C₁-C₄-alkylsulfonyl, benzylsulfonyl, —(CH₂)ᵣCOR¹⁹, —(CH₂)ₛCONR²⁰R²¹, —(CH₂)ₜNR²⁸COR²⁹ or —(CH₂)_{w}NR³⁰SO₂R³¹,
   whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²³,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
      wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
         wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxy-sulfonyl, C₁-C₄-alkyl, C₁-C₄-alkoxy, C₁-C₄-alkylamino, C₁-C₄-alkylsulfonyl and C₁-C₄-alkoxycarbonyl,
   and
   R²³ represents C₃-C₇-cycloalkyl, phenyl, 5- to 10-membered heterocyclyl or 5- to 10-membered heteroaryl,
      wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
r represents a number 0, 1, 2 or 3,
s represents a number 0, 1, 2 or 3,
t represents a number 2 or 3,
w represents a number 2 or 3,
R¹⁹ represents C₁-C₆-alkyl, C₂-C₄-alkenyl, C₁-C₆-alkoxy, phenyl, benzyloxy or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²⁰ represents hydrogen, C₁-C₄-alkyl or phenyl,
   whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
      wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²¹ represents hydrogen or C₁-C₄-alkyl,
R²⁸ represents hydrogen or C₁-C₄-alkyl,
R²⁹ represents C₁-C₆-alkyl, C₂-C₄-alkenyl, C₁-C₆-alkoxy, phenyl, benzyloxy or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R³⁰ represents hydrogen or C₁-C₄-alkyl,
R³¹ represents C₁-C₆-alkyl, C₂-C₄-alkenyl, phenyl or 5- to 10-membered heterocyclyl,
   whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
      wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,

R⁸ represents hydrogen, oxo, trifluoromethyl, trifluoromethoxy, C₁-C₄-alkyl, C₁-C₄-alkoxy or C₁-C₄-alkylthio,
R⁹ represents hydrogen, C₁-C₄-alkyl or C₁-C₄-alkoxy,
R¹⁰ represents hydrogen or C₁-C₄-alkyl
R¹¹ represents hydrogen or C₁-C₄-alkyl
R¹² represents hydrogen or C₁-C₄-alkyl
R¹³ represents hydrogen or C₁-C₄-alkyl

R³ represents hydrogen, halogen, cyano, methyl, ethyl, methoxy, ethoxy or phenoxy,

R⁴ represents hydrogen, halogen, methyl, ethyl, methoxy or ethoxy,

R⁵ represents hydroxy, amino, trifluoromethyl, trifluoromethoxy, hydroxycarbonyl, aminocarbonyl, hydroxymethyl, aminomethyl, C₁-C₄-alkylamino, C₁-C₄-alkylcarbonyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonylamino, C₂-C₄-alkenylsulfonylamino, C₁-C₄-alkylsulfonyl(C₁-C₄-alkyl)amino, benzylsulfonylamino, 5- or 6-membered heteroarylsulfonylamino or 5- to 7-membered heterocyclyl,
whereby alkylaminocarbonyl, alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino,

R⁶ represents hydrogen, halogen, C₁-C₄-alkyl or C₁-C₄-alkoxy,

R⁷ represents hydrogen, halogen, C₁-C₄-alkyl or C₁-C₄-alkoxy,

or

R⁵ and R⁶ are bonded to neighboring carbon atoms and together with the carbon atoms to which they are bonded form a 1,3-dioxolane,

and their salts, their solvates and the solvates of their salts.

The invention also relates to compounds of formula (I) in which

R¹ and R² together with the carbon atom to which it is bonded form a group of formula

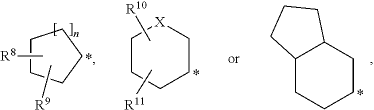

whereby

* represents the carbon atom to which R¹ and R² are bonded,
n represents the number 2,

X represents an oxygen atom, a sulfur atom or NR¹⁴,
whereby
R¹⁴ represents C₁-C₆-alkyl, C₂-C₄-alkenyl, C₁-C₄-alkylsulfonyl, benzylsulfonyl, —(CH₂)ₒCOR¹⁶ or —(CH₂)ₚCONR¹⁷R¹⁸
   whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₃-C₇-cycloalkyl, phenyl, 5- to 10-membered heterocyclyl and 5- to 10-membered heteroaryl,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   and
   whereby
   o represents a number 0, 1, 2 or 3,
   p represents a number 0, 1, 2 or 3,
   R¹⁶ represents C₁-C₆-alkyl, C₂-C₄-alkenyl, C₁-C₆-alkoxy, phenyl, benzyloxy or 5- to 10-membered heterocyclyl,
      whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
         wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   R¹⁷ represents hydrogen, C₁-C₄-alkyl or phenyl,
      whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
         wherein phenyl and heteroaryl for their part can be substituted with 1 to 3 substituents, whereby the substituents are selected independently of one another from the group consisting of halogen, cyano, trifluoromethyl, trifluoromethoxy and C₁-C₄-alkyl,
   R¹⁸ represents hydrogen or C₁-C₄-alkyl,

R⁸ represents hydrogen, oxo, trifluoromethyl, trifluoromethoxy, C₁-C₄-alkyl, C₁-C₄-alkoxy or C₁-C₄-alkylthio,
R⁹ represents hydrogen, C₁-C₄-alkyl or C₁-C₄-alkoxy,
R¹⁰ represents hydrogen,
R¹¹ represents hydrogen,

R³ represents hydrogen, halogen, methyl, ethyl, methoxy, ethoxy or phenoxy,

R⁴ represents hydrogen, halogen, methyl, ethyl, methoxy or ethoxy,

R⁵ represents hydroxy, amino, trifluoromethyl, trifluoromethoxy, hydroxycarbonyl, aminocarbonyl, hydroxymethyl, aminomethyl, C₁-C₄-alkylamino, C₁-C₄-alkylcarbonyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonylamino, C₂-C₄-alkenylsulfonylamino, C₁-C₄-alkylsulfonyl(C₁-C₄-alkyl)amino, benzylsulfonylamino, 5- or 6-membered heteroarylsulfonylamino or 5- to 7-membered heterocyclyl,
whereby alkylaminocarbonyl, alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino,

R⁶ represents hydrogen, halogen, C₁-C₄-alkyl or C₁-C₄-alkoxy,

R⁷ represents hydrogen,

or

R⁵ and R⁶ are bonded to neighboring carbon atoms and together with the carbon atoms to which they are bonded form a 1,3-dioxolane.

and their salts, their solvates and the solvates of their salts.

The invention also relates to compounds of formula (I) in which

R¹ and R² together with the carbon atom to which they are bonded form a group of formula

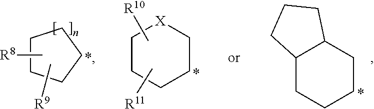

whereby
* represents the carbon atom to which R¹ and R² are bonded,
n represents the number 2,
X represents NR¹⁴,
   whereby
R¹⁴ represents C₁-C₄-alkyl, C₂-C₄-alkenyl, benzylsulfonyl, —(CH₂)ₒCOR¹⁶ or —(CH₂)ₚCONR¹⁷R¹⁸,
   whereby alkyl and alkenyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₃-C₇-cycloalkyl, phenyl, 5- to 10-membered heterocyclyl and 5- to 10-membered heteroaryl,
      wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
   and
   whereby
   o represents a number 1 or 2,
   p represents a number 1 or 2,
   R¹⁶ represents C₁-C₄-alkyl, C₁-C₄-alkoxy, phenyl or benzyloxy,
   R¹⁷ represents hydrogen, C₁-C₄-alkyl or phenyl,
      whereby alkyl can be substituted with a substituent, whereby the substituent is selected from the group consisting of methoxy, phenyl and 5- or 6-membered heteroaryl,
         wherein phenyl for its part can be substituted with 1 to 3 substituents, whereby the substituents are selected independently of one another from the group consisting of halogen, cyano, trifluoromethyl, trifluoromethoxy and C₁-C₄-alkyl,
   R¹⁸ represents hydrogen,
R⁸ represents hydrogen, C₁-C₄-alkyl or C₁-C₄-alkoxy,
R⁹ represents hydrogen or C₁-C₄-alkyl,
R¹⁰ represents hydrogen,
R¹¹ represents hydrogen,

R³ represents hydrogen, halogen, methyl, ethoxy or phenoxy,

R⁴ represents hydrogen, halogen or methyl,

R⁵ represents hydroxy, trifluoromethyl, trifluoromethoxy, hydroxycarbonyl, aminocarbonyl, hydroxymethyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonylamino, C₂-C₄-alkenylsulfonylamino, C₁-C₄-alkylsulfonyl(C₁-C₄-alkyl)amino, benzylsulfonylamino or 5- or 6-membered heteroarylsulfonylamino,
whereby alkylaminocarbonyl, alkylcarbonylamino and alkylsulfonylamino can be substituted with a substituent, whereby the substituent is selected from the group consisting of amino, C₁-C₄-alkylamino, morpholinyl and pyrrolidinyl,

R⁶ represents hydrogen, halogen, C₁-C₄-alkyl or C₁-C₄-alkoxy,

R⁷ represents hydrogen,

and their salts, their solvates and the solvates of their salts.

The invention also relates to compounds of formula (I) in which R⁵ represents hydroxy, amino, hydroxycarbonyl, aminocarbonyl, hydroxymethyl, aminomethyl, C₁-C₄-alkylamino, C₁-C₄-alkylcarbonyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonylamino, C₂-C₄-alkenylsulfonylamino, C₁-C₄-alkylsulfonyl(C₁-C₄-alkyl)amino, benzylsulfonylamino, 5- or 6-membered heteroarylsulfonylamino or 5- to 7-membered heterocyclyl,

whereby alkylaminocarbonyl, alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino.

The invention also relates to compounds of formula (I) in which R⁵ represents C₁-C₄-alkylcarbonylamino or C₁-C₄-alkylsulfonylamino,

whereby alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino.

The invention also relates to compounds of formula (I) in which R⁵ represents C₁-C₄-alkylsulfonylamino,

whereby alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino.

The invention also relates to compounds of formula (I) in which R⁵ represents C₁-C₄-alkylsulfonylamino,

whereby alkylsulfonylamino can be substituted with a substituent, whereby the substituent is selected from the group consisting of amino, C₁-C₄-alkylamino, morpholinyl and pyrrolidinyl.

The invention also relates to compounds of formula (I) in which R⁵ represents C₁-C₄-alkylsulfonyl.

The invention further relates to a method for the preparation of the compounds of formula (I), whereby according to method

[A] compounds of formula

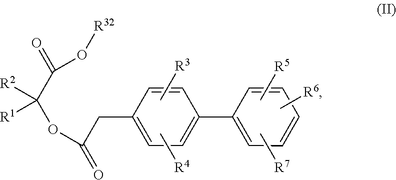

in which

R¹, R², R³, R⁴, R⁵, R⁶ and R⁷ have the meaning indicated above, and

R³² represents methyl or ethyl,

are reacted with a base,

or

[B] compounds of formula

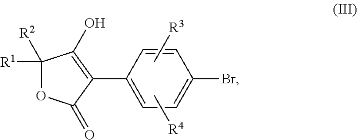

in which

R¹, R², R³ and R⁴ have the meaning indicated above,

are reacted under Suzuki coupling conditions with compounds of formula

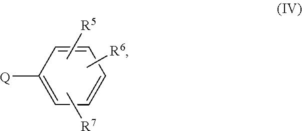

in which

R⁵, R⁶ and R⁷ have the meaning indicated above, and

Q represents —B(OH)₂, a boronic acid ester, preferably boronic acid pinacol ester, or —BF₃⁻K⁺.

If compounds with free amino functions are formed in the reactions according to method [A] or method [B] these amino functions can be reacted with carboxylic acids, carboxylic acid chlorides, alkyl halides, benzyl halides or sulfonyl chlorides by reaction methods known to the skilled person and further compounds of formula (I) can be prepared this way.

The reaction according to method [A] generally takes place in inert solvents, preferably in a temperature range from room temperature to the reflux of the solvent under atmospheric pressure.

Inert solvents are, for example, hydrocarbons such as toluene or benzene, or other solvents such as dioxan, dimethylformamide or acetonitrile. It is also possible to use mixtures of the solvents. Dimethylformamide is particularly preferred.

Bases are, for example, potassium tert.-butylate, sodium hydride, lithium diisopropylamide, sodium, potassium or lithium hexamethyldisilylamide. Potassium tert.-butylate is particularly preferred.

The reaction according to method [B] generally takes place in inert solvents in the presence of a catalyst, optionally in the presence of an auxiliary, preferably in a temperature range from room temperature to 130° C. under atmospheric pressure.

Catalysts are, for example, palladium catalysts usual for Suzuki reaction conditions, catalysts such as, for example, dichlorobis(triphenylphosphine) palladium, tetrakistriphenylphosphine palladium(0), palladium(II) acetate, palladium(II) acetate/triscyclohexylphosphine or bis(diphenylphosphaneferrocenyl)palladium(II) chloride or palladium(II) acetate with a ligand such as dicyclohexyl(2′,4′,6′-triisopropylbiphenyl-2-yl)phosphine are preferred.

Auxiliaries are, for example, potassium acetate, cesium, potassium or sodium carbonate, potassium tert.-butylate, cesium fluoride or potassium phosphate performed, auxiliaries such as, for example, potassium acetate and/or an aqueous sodium carbonate solution are preferred.

Inert solvents are, for example ethers such as dioxan, tetrahydrofuran or 1,2-dimethoxyethane, hydrocarbons such as benzene, xylene or toluene, or carboxamides such as dimethylformamide or dimethylacetamide, alkylsulfoxides such as dimethylsulfoxide, or N-methylpyrrolidone, or mixtures of the solvents with alcohols such as methanol or ethanol and/or water, 1,2-dimethoxyethane is preferred.

Compounds of formula (III) may be synthesized by method [A] from the corresponding starting materials.

Compounds of formula (IV) are known or may be synthesized by known methods from the corresponding starting materials.

Compounds of formula (II) are known or can be prepared by reacting compounds of formula

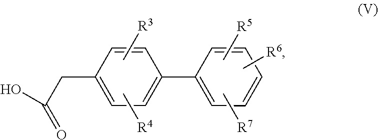

in which

R³, R⁴, R⁵, R⁶ and R⁷ have the meaning indicated above,

in first stage with thionyl chloride or oxalyl chloride and in the second stage with a compound of formula

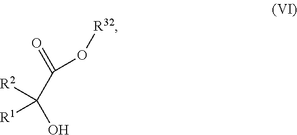

in which

R¹, R² and R³² have the meaning indicated above.

The reaction of the compound of formula (V) with thionyl chloride or oxalyl chloride in the first stage generally takes place in an inert solvent, preferably in a temperature range from room temperature to the reflux of the solvent under atmospheric pressure.

Inert solvents are, for example, halohydrocarbons such as dichloromethane or dichloroethane, hydrocarbons such as benzene, xylene or toluene or other solvents such as chlorobenzene, toluene is preferred.

The reaction of the resulting acid chloride with a compound of formula (VI) in the second stage generally takes place in inert solvents, preferably in a temperature range from 50° C. to the reflux of the solvent under atmospheric pressure.

Inert solvent are, for example, hydrocarbons such as benzene, xylene or toluene, or other solvents such as chlorobenzene, toluene is preferred.

The compounds of formulae (V) and (VI) are known or may be synthesized by known methods from the corresponding starting materials.

In an alternative method the reaction of the compounds of formula (V) with compounds of formula (VI) can also proceed via the thiocarbonic esters of the compounds of formula (V).

The preparation of the compounds of the invention can be illustrated by the following synthesis scheme.

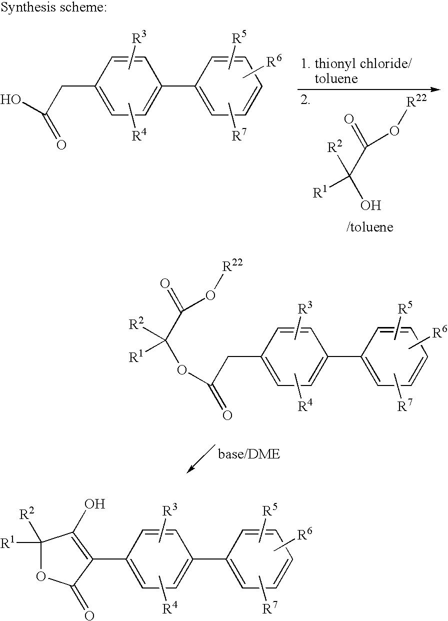

The compounds of the invention show a valuable spectrum of pharmacological activity that could not have been predicted.

They are therefore suitable for use as medicament for the treatment and/or prophylaxis of diseases in humans and animals.

The compounds of the present invention are characterized in particular by an advantageous anti-retroviral spectrum of activity.

The present invention further relates to the use of the compounds of the invention for the treatment and/or prophylaxis of diseases that are caused by retroviruses, in particular HI viruses.

The present invention further relates to the use of the compounds of the invention for the treatment and/or prophylaxis of diseases, in particular the previously named diseases.

The present invention further relates to the use of the compounds of the invention for the manufacture of a medicament for the treatment and/or prophylaxis of diseases, in particular the previously named diseases.

The present invention further relates to a method for the treatment and/or prophylaxis of diseases, in particular the previously named diseases, using a therapeutically effective amount of the compounds of the invention.

Areas of indication in human medicine which may be mentioned by way of example are:
1.) The treatment and prophylaxis of human retrovirus infections.
2.) For the treatment and prophylaxis of HIV I-(virus of human immune deficiency; formerly called HTLV III/LAV) and HIV II-induced infections and diseases (AIDS) and the stages associated therewith such as ARC (AIDS related complex) and LAS (lymphadenophathy syndrome) and the immune deficiency and encephalopathy caused by this virus.
3.) For the treatment of HIV infections caused by single-, multiple- or multiresistant HIV viruses.

Resistant HI viruses means for example, viruses with resistances towards nucleosidic inhibitors (RTI), non-nucleosidic inhibitors (NNRTI) or protease inhibitors (PI) or viruses with resistances towards other activity principles, e.g., T20 (fusion inhibitors).
4.) For the treatment or prophylaxis of the AIDS carrier state.
5.) For the treatment or prophylaxis of an HTLV-I or HTLV-II infection

Indications in veterinary medicine which may be mentioned by way of example are:

Infections with
a) Maedivisna (in sheep and goats)
b) progressive pneumonia virus (PPV) (in sheep and goats)
c) caprine arthritis encephalitis virus (in sheep and goats)
d) Zwoegerziekte virus (in sheep)
e) infectious anemia virus (of the horse)
f) infections caused by the feline leukemia virus
g) infections caused by the feline immune deficiency virus (FIV)
h) infections caused by the simian immune deficiency virus (SIV)

Points 2, 3 and 4 listed above are preferred in the areas of indication in the human medicine.

The present invention further relates to medicaments comprising at least one compound of the invention and at least one or more further active substances, in particular for the treatment and/or prophylaxis of the previously named diseases.

The compounds of the invention can also be used advantageously, particularly in the points 2, 3 and 4 listed above, as components of a combination therapy with one or more other compounds active in these therapeutic areas. For example, these compounds can be used in combination with effective doses of antivirally active substances which are based on the activity principles listed below:

HIV protease inhibitors; named by way of example: saquinavir, indinavir, ritonavir, nelfinavir, amprenavir, tipranavir;

Nucleosidic and non-nucleosidic inhibitors of the HIV reverse transcriptase; named by way of example: zidovudin, lamivudin, didanosin, zalzitabin, stavudin, abacavir, tenofovir, adefovir, nevirapin, delavirdin, efavirenz, emtricitabin, etravirin, rilpivirin;

HIV integrase inhibitors named by way of example: S1360, L870810;

HIV fusion inhibitors; named by way of example: pentafuside, T1249.

Cytochrome P450 Monooxygenase Inhibitors; Named by Way of Example: Ritonavir.

This selection is to illustrate the combination possibilities, not, however, to restrict to the examples listed here; in principle every combination of the compounds of the invention with antivirally active substances is to be considered within the scope of the invention.

The compounds of the invention can act systemically and/or locally. For this purpose they can be applied in a suitable way, such as for example, orally, parenterally, pulmonally, nasally, sublingually, lingually, buccally, rectally, dermally, transdermally, conjunctivally, otically or as an implant or stent.

For these administration routes the compounds of the invention can be administered in suitable administration forms.

Suitable for oral administration are administration forms which function according to the prior art and release the compounds of the invention rapidly and/or in modified fashion and which contain the compounds of the invention in crystalline and/or amorphous and/or dissolved form, e.g., tablets (uncoated or coated tablets, for example having coatings which are resistant to gastric juice or dissolve with a delay or are insoluble and control the release of the compounds of the invention), tablets or films/wafers which disintegrate rapidly in the oral cavity, films/lyophilisates, capsules (for example hard or soft gelatin capsules), sugar coated tablets, granules, pellets, powders, emulsions, suspensions, aerosols or solutions.

Parenteral administration can take place with avoidance of an absorption step (e.g., intravenously, intraarterially, intracardially, intraspinally or intralumbally) or with inclusion of an absorption (e.g., intramuscularly, subcutaneously, intracutaneously, percutaneously or intraperitoneally). Administration forms suitable for parenteral administration are i.a. preparations for injection and infusion in the form of solutions, suspensions, emulsions, lyophilisates or sterile powders.

Suitable for other administration routes are, for example, pharmaceutical forms for inhalation (i.a. powder inhalators, nebulizers), nasal drops, solutions, sprays; tablets, films/wafers or capsules for lingual, sublingual or buccal administration, suppositories, preparations for ears or eyes, vaginal capsules, aqueous suspensions (lotions, shaking mixtures), lipophilic suspensions, ointments, creams, transdermal therapeutic systems (for example, plasters), milk, pastes, foams, dusting powders, implants or stents.

The compounds of the invention can be converted into the stated administration forms. This can take place in a manner known per se by mixing with inert, non-toxic, pharmaceutically acceptable excipients. These excipients include i.a. carriers (e.g., microcrystalline cellulose, lactose, mannitol), solvents (e.g., liquid polyethyleneglycols), emulsifiers and dispersants or wetting agents (for example, sodium dodecyl sulfate, polyoxysorbitanoleate), binding agents (for example polyvinylpyrrolidone), synthetic and natural polymers (for example, albumin), stabilizers (e.g., antioxidants such as for example ascorbic acid), colors (e.g., inorganic pigments such as for example iron oxides) and taste and/or odor corrigents.

The present invention further relates to medicaments, which comprise at least one compound of the invention, usually together with one or more inert, non-toxic, pharmaceutically acceptable excipients, and to their use for the previously described purposes.

In general it has proved advantageous in both human and veterinary medicine to administer the active compound(s) of the invention in total amounts of 0.1 to 200 mg/kg, preferably 1 to 100 mg/kg of body weight every 24 hours, where appropriate in the form of several individual doses to achieve the desired results. A single dose contains the active compound(s) in amounts of 1 to 80 mg/kg, in particular 1 to 30 mg/kg body weight.

It may nevertheless be necessary where appropriate to deviate from the amounts mentioned, in particular depending on body weight, administration route, individual behavior towards to the active ingredient, nature of the preparation and time or interval over which administration takes place. Thus it may be sufficient in some cases to make do with less than the aforementioned minimum amount, whereas in other cases the stated upper limit must be exceeded. In the case of an administration of larger amounts it may be advisable to divide these into a plurality of individual doses over the day.

The percentage data in the following tests and examples are percentages by weight, unless otherwise stated, parts are parts by weight. Solvent ratios, dilution ratios and concentration data of liquid/liquid solutions are in each case based on volume. The statement “w/v” means “weight/volume”. Thus, for example “10% w/v” means that 100 ml of solution or suspension contain 10 g of substance.

### A) EXAMPLES

### Abbreviations

aq. aqueous, aqueous solution
conc. concentrated
DCI direct chemical ionization (in MS)
DCM dichloromethane
DIPEA diisopropylethylamine

### DMA N,N-dimethylacetamide

DME dimethoxyethane

### DMF N,N-dimethylformamide

DMSO dimethylsulfoxide
EDC N′-(3-dimethylaminopropyl)-N-ethylcarbodiimide×HC1
eq. equivalent(s)
ESI electrospray ionization (in MS)
GWP general working procedure
h hour(s)
HATU O-(7-azabenzotriazol-1-yl)-N,N,N′,N′-tetramethyluronium hexafluorophosphate
HPLC high pressure, high performance liquid chromatography
LC-MS liquid chromatography-coupled mass spectrometry
min minute(s)
MS mass spectrometry
NMR nuclear magnetic resonance spectroscopy
PyBOP benzotriazol-1-yloxytris(pyrrolidino)phosphonium hexafluorophosphate
Rₜ retention time (in HPLC)
RT room temperature
TBTU O-(benzotriazol-1-yl)-N,N,N′,N′-tetramethyluronium tetrafluoroborate
TFA trifluoroacetic acid
th. of theory (with yields)
THF tetrahydrofuran
TMOF trimethylorthoformate

LC-MS and HPLC Methods:

Method 1 (LC-MS): Instrument: Micromass Quattro LCZ with HPLC Agilent Series 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

Method 2 (LC-MS): MS Instrument type: Micromass ZQ; HPLC Instrument type: Waters Alliance 2795; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 3 (LC-MS): MS Instrument type: Micromass ZQ; HPLC Instrument type: HP 1100 Series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.

Method 4 (LC-MS): Instrument: Micromass Platform LCZ with HPLC Agilent Series 1100; column: Thermo Hypersil GOLD 3μ 20 mm×4 mm; eluent A: 1 l of water+0.5 ml of 50% formic acid, eluent B: 1 l of acetonitrile+0.5 ml of 50% formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

Method 5 (LC-MS): Instrument: Micromass Quattro LCZ with HPLC Agilent Series 1100; column: Phenomenex Onyx Monolithic C18, 100 mm×3 mm. eluent A: 1 l of water+0.5 ml of 50% formic acid, eluent B: 1 l of acetonitrile+0.5 ml of 50% formic acid; gradient: 0.0 min 90% A→2 min 65% A→4.5 min 5% A→6 min 5% A; flow rate: 2 ml/min; oven: 40° C.; UV detection: 208-400 nm.

Method 6 (LC-MS): MS Instrument type: Micromass ZQ; HPLC Instrument type: HP 1100 Series; UV DAD; column: Phenomenex Gemini 3μ 30 mm×3.00 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min. 2 ml/min; oven: 50° C.; UV detection: 210 nm.

Method 7 (LC-MS): MS Instrument type: Waters ZQ; HPLC instrument type: Waters Alliance 2795; column: Phenomenex Onyx Monolithic C18, 100 mm×3 mm; eluent A: 1 l of water+0.5 ml of 50% formic acid, eluent B: 1 l of acetonitrile+0.5 ml of 50% formic acid; gradient: 0.0 min 90% A→2 min 65% A→4.5 min 5% A→6 min 5% A; flow rate: 2 ml/min; oven: 40° C.; UV detection: 210 nm.

GC/MS Methods:

Method 1 (GC-MS): Instrument: Micromass GCT, GC6890; column: Restek RTX-35MS, 30 m×250 μm×0.25 μm; constant flow with helium: 0.88 ml/min; oven: 60° C.; inlet: 250° C.; gradient: 60° C. (hold for 0.30 min), 50° C./min→120° C., 16° C./min→250° C., 30° C./min→300° C. (hold for 1.7 min).

Enantiomer Separation:

Method 1 (HPLC, chiral): Column: Daicel Chiralpak AS-H, 250 mm×20 mm, 5 μm; eluent: 1:1 iso-hexane:ethanol/0.2% glacial acetic acid/1% water; oven: 50° C.; flow rate: 15 ml/min; UV detection: 220 nm.

Starting Compounds:

### Example 1A

### Methyl 1-benzyl-4-hydroxypiperidine-4-carboxylate

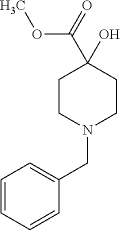

A solution of 10.52 g (48.64 mmol) of 1-benzyl-4-hydroxypiperidine-4-carbonitrile in 60 ml of conc. hydrochloric acid is stirred for one hour at 90° C. The reaction solution is concentrated on a rotary evaporator and dried under high vacuum. The residue obtained is taken up in 150 ml of methanol, 6 ml of conc. sulfuric acid are added and the mixture stirred for 1 hour at 50° C. After cooling the reaction mixture is diluted with ethyl acetate and rendered alkaline with a saturated sodium carbonate solution. The organic phase is washed with a sodium chloride solution, dried over sodium sulfate and concentrated on a rotary evaporator. 10.8 g (43.6 mmol, 90% th.) of product are obtained. LC-MS (method 4): Rₜ=2.08 min. MS (ESIpos): m/z=250 (M+H)^{+ 1}H NMR (400 MHz, DMSO-d₆): δ=7.35-7.2 (m, 5H), 5.28 (s, 1H), 3.63 (s, 3H), 3.45 (s, 2H), 2.53-2.4 (m, 2H, partly masked by DMSO), 2.38-2.2 (m, 2H), 1.9-1.78 (m, 2H), 1.59 (d, 2H).

### Example 2A

### Methyl 3-hydroxypiperidine-3-carboxylate

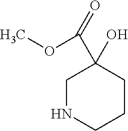

1.08 g (1.02 mmol) of palladium on activated carbon 10% and 12.84 g (203.6 mmol) of ammonium formate are added to a solution of 9 g (33.9 mmol) of methyl 1-benzyl-3-hydroxypiperidine-3-carboxylate in 100 ml of ethanol and 100 ml of ethyl acetate and the mixture is stirred for 3 hours at 80° C. After cooling the reaction solution is filtered over silica gel and washed with ethanol. The silica gel/product mixture is stirred with a solution of ethanol/ammonia 20:1, filtered with suction and the filtrate is concentrated on a rotary evaporator. 2.19 g (13.8 mmol, 57% th.) of product are obtained. GC-MS (Method 1): Rₜ=5.43 min. MS (ESIpos): m/z=159 (M+H)^{+ 1}H NMR (300 MHz, DMSO-d₆): δ=3.6 (s, 3H), 2.7-2.45 (m, 4H, partly masked by DMSO), 1.95-1.8 (m, 1H), 1.65-1.5 (m, 2H), 1.4-1.27 (m, 1H).

### Example 3A

### Methyl 4-hydroxypiperidine-4-carboxylate

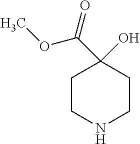

Starting from 15.5 g (62.2 mmol) of methyl 1-benzyl-4-hydroxypiperidine-4-carboxylate from example 1A, 0.662 g (0.62 mmol) of palladium on activated charcoal and 11.76 g (186.5 mmol) of ammonium formate 9.68 g (60.8 mmol, 98% th.) of product are obtained according to the method described in example 2A. GC-MS (Method 1): Rₜ=5.59 min. MS (ESIpos): m/z=160. ¹H NMR (300 MHz, DMSO-d₆): δ=3.67 (s, 3H), 2.86-2.73 (m, 2H), 2.73-2.6 (m, 2H), 1.85-1.7 (m, 2H), 1.5 (d, 2H).

### Example 4A

### 1-Benzyl-3-methyl-3-hydroxypiperidine-1,3-dicarboxylate

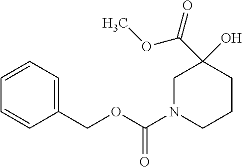

15.57 ml (89.4 mmol) of N,N-diisopropylethylamine are added to a solution of 5.27 g (29.8 mmol) of methyl 3-hydroxypiperidine-3-carboxylate from example 2A in 100 ml of DMF. With ice cooling a solution of 6.1 g (35.75 mmol) of benzyl chloroformate in 50 ml of DMF is added dropwise. Stirring is continued for 2 hours at room temperature. The reaction mixture is diluted with water and extracted with dichloromethane. The organic phase is washed with 1 molar hydrochloric acid and with a sat. sodium chloride solution, dried over sodium sulfate and concentrated on a rotary evaporator. The residue obtained is separated by preparative HPLC. The product mixture obtained is dissolved in 200 ml of methanol, conc. sulfuric acid is added and the mixture is stirred overnight under reflux. After cooling the reaction mixture is concentrated on a rotary evaporator and dried under high vacuum. 4.9 g (16.7 mmol, 54% th.) of product are obtained. LC-MS (Method 3): Rₜ=1.98 min. MS (ESIpos): m/z=294 (M+H)⁺. ¹H NMR (400 MHz, DMSO-d₆): δ=7.4-7.27 (m, 5H), 5.68-5.53 (m, 1H), 5.13-4.95 (m, 2H), 3.72-3.48 (m, 4H), 3.4-3.25 (m, 2H, partly masked by water), 3.2-3.05 (m, 1H), 1.92-1.75 (m, 1H), 1.75-1.6 (m, 2H), 1.5-1.48 (m, 1H).

### Example 5A

### 1-Benzyl-4-methyl-4-hydroxypiperidine-1,4-dicarboxylate

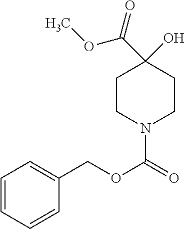

Starting from 9.7 g (60.75 mmol) of methyl 4-hydroxypiperidine-4-carboxylate from example 3A and 11.4 g (66.82 mmol) of benzyl chloroformate 8.27 g (28.2 mmol, 45% th.) of product are obtained according to the method described in example 4 and after purification on a silica gel column (eluent: cyclohexane/ethyl ester 1:1). LC-MS (Method 2): Rₜ=1.7 min. MS (ESIpos): m/z=294 (M+H)^{+ 1}H NMR (400 MHz, DMSO-d₆): δ=7.4-7.28 (m, 5H), 5.6 (s, 1H), 5.08 (s, 2H), 3.8-3.7 (m, 2H), 3.64 (s, 3H), 3.25-3.08 (m, 2H), 1.8-1.7 (m, 2H), 1.6 (d, 2H).

### Example 6A

### (1-Ethoxycarbonylcyclohexyl) 4-bromo-5-chloro-2-methylphenylacetate

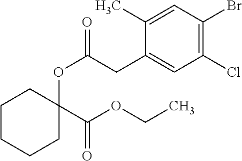

3.00 g (11.4 mmol) of 4-bromo-5-chloro-2-methylphenylacetic acid (example XXIII-8 from WO 97/01535) are provided in 30 ml of toluene, 2.5 ml (34.3 mmol) of thionyl chloride are added and the mixture is stirred for 7 hours at 80° C. until hydrogen chloride generation has ceased. After cooling the mixture is concentrated and the acid chloride generated is heated for two days under reflux with 1.96 g (11.4 mmol) of ethyl 1-hydroxy-cyclohexanecarboxylate in 30 ml of toluene. The mixture is concentrated and the residue is purified by flash chromatography (eluent: cyclohexane/ethyl ester 95:5). 4.20 g (88% th.) of product are obtained. LC-MS (Method 1): Rₜ=3.26 min. MS (ESIpos): m/z 417 (M+H)⁺. ¹H NMR (300 MHz, DMSO-d₆): δ=7.63 (s, 1H), 7.52 (s, 1H), 4.04 (q, 2H), 3.78 (s, 2H), 2.23 (s, 3H), 2.02-1.92 (m, 2H), 1.75-1.63 (m, 2H), 1.59-1.49 (m, 3H), 1.45-1.20 (m, 3H), 1.10 (t, 3H).

GWP1: Esterification

The phenylacetic acid is provided in toluene, thionyl chloride (3 eq.) is added and the mixture is stirred at 80° C. until hydrogen chloride generation has ceased. After cooling the mixture is concentrated and the acid chloride obtained is heated under reflux for two days with the hydroxycarboxylic acid ester in toluene. The mixture is concentrated and purified or where appropriate diastereomers are separated by flash chromatography (eluent: cyclohexane/ethyl acetate gradient). Alternatively the purification or diastereomer separation can take place by column chromatography on silica gel 60 (eluent: cyclohexane/ethyl acetate gradient) or by preparative HPLC (RP18 column, eluent: acetonitrile-water gradient, 0.1% formic acid).

GWP2:

The phenylacetic acid is provided in toluene and oxalyl chloride (5 eq.) is added and the mixture is stirred at 80° C. until hydrogen chloride generation has ceased. After cooling the mixture is concentrated and the acid chloride formed is heated overnight with the hydroxycarboxylic acid ester in toluene at 140° C. The mixture is concentrated and purified or where appropriate diastereomers are separated by flash chromatography (eluent: cyclohexane/ethyl acetate gradient). Alternatively purification or diastereomer separation can take place by column chromatography on silica gel 60 (eluent: cyclohexane/ethyl acetate gradient) or by preparative HPLC (RP18 column, eluent: acetonitrile-water gradient, 0.1% formic acid).

### Example 7A

### (4-Bromo-2-ethoxy-5-methylphenyl)acetic acid

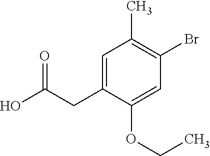

1 g (4.05 mmol) of (4-bromo-2-fluoro-phenyl)acetic acid is heated in 12 ml of a 21% solution of sodium ethylate in ethanol in a microwave for 3 h at 180° whereby a pressure of about 14 bar is generated. After cooling a sat. sodium chloride solution is added and the mixture is extracted three times with ethyl acetate. The combined organic phases are dried over sodium sulfate and concentrated in vacuum. The residue is purified by preparative HPLC (RP18 column; eluent: acetonitrile-water gradient, 0.1% formic acid). Yield: 565 mg (49% th.) of crystals. LC-MS (Method 1): Rₜ=2.10 min. MS (ESIneg): m/z=271 (M−H)⁺. ¹H NMR (300 MHz, DMSO-d₆): δ=12.2 (b, 1H), 7.04 (s, 1H), 7.02 (s, 1H), 4.0 (q, 2H), 3.43 (s, 2H), 2.24 (s, 3H), 1.26 (t, 3H).

The following compounds are prepared in analogy to example 6A, the respective GWP and the general preparative information. The phenylacetic acids are known in part from WO 97/01535 or WO 99/55673 or are prepared in analogy thereto, the hydroxycarboxylic acid esters can be obtained from the corresponding cyanohydrins according to T. Bretschneider, J. Benet-Buchholz, R. Fischer, R. Nauen, Chimia 2003, 57, 697-701.

| | | Prepared | Analysis |
|---|---|---|---|
| | | according to | LC-MS (Method) |
| Ex. | | GWP | Rₜ [min] |
| No. | Structure | Yield [th.] | MS: m/z |
| 8A | 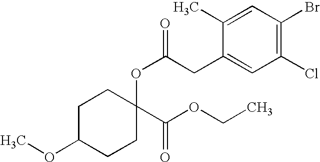 | GWP 2007-06-15-1- 44% | LC-MS (3) 3.03, 3.10 ESI+: 447 (M + H)⁺ |
| 9A | 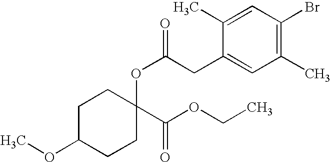 | GWP 2007-06-15-1- 75% | LC-MS (2) 2.82, 2.90 ESI+: 427 (M + H)⁺ |
| 10A | 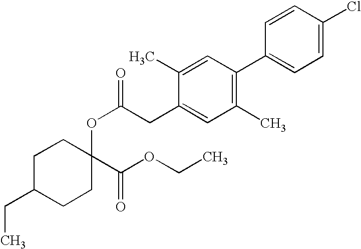 | GWP 2007-06-15-1- 61% | LC-MS (3) 3.59 ESI+: 457 (M + H)⁺ |
| 11A | 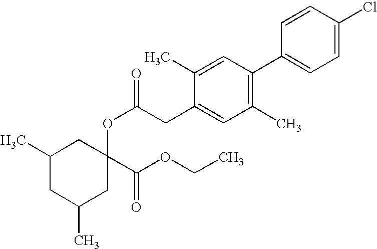 | GWP 2007-06-15-1- 53% | LC-MS (3) 3.58 ESI+: 457 (M + H)⁺ |
| 12A | 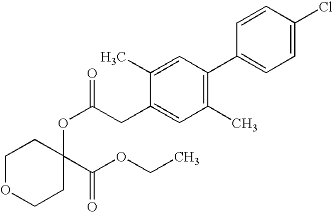 | GWP 2007-06-15-1- 31% | LC-MS (1) 3.18 ESI+: 431 (M + H)⁺ |
| 13A | 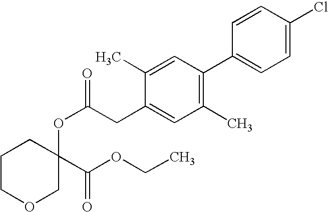 | GWP 2007-06-15-1- 48% | LC-MS (1) 3.21 ESI+: 431 (M + H)⁺ |
| 14A | 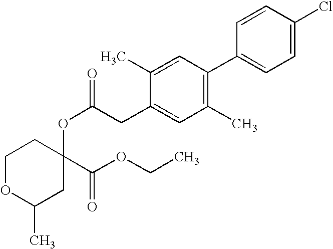 | GWP 2007-06-15-1- 46% | LC-MS (1) 3.28 ESI+: 445 (M + H)⁺ |
| 15A | 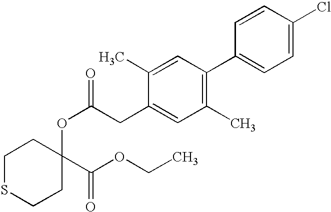 | GWP 2007-06-15-1- 48% | LC-MS (1) 3.35 ESI+: 447 (M + H)⁺ |
| 16A | 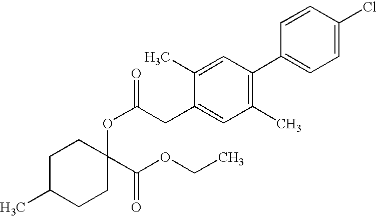 | GWP 2007-06-15-1- 43% | LC-MS (1) 3.51 ESI+: 443 (M + H)⁺ |
| 17A | 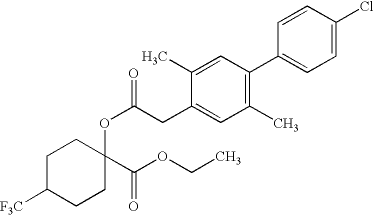 | GWP 2007-06-15-1- 58% | LC-MS (1) 3.28 ESI+ : 445 (M + H)⁺ |
| 18A | 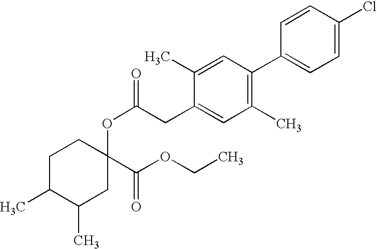 | GWP 2007-06-15-1- 46% | LC-MS (1) 3.56 ESI+: 457 (M + H)⁺ |
| 19A | 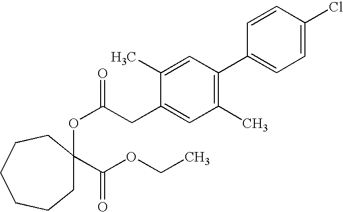 | GWP 2007-06-15-1- 56% | LC-MS (1) 3.50 ESI+: 443 (M + H)⁺ |
| 20A | 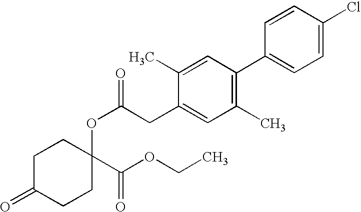 | GWP 2007-06-15-1- 49% | LC-MS (1) 3.11 ESI+: 443 (M + H)⁺ |
| 21A | 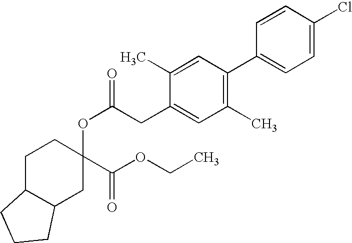 | GWP 2007-06-15-1- 33% | LC-MS (1) 3.60 ESI+: 469 (M + H)⁺ |
| 22A | 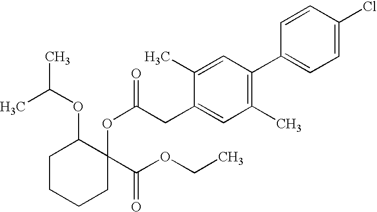 | GWP 2007-06-15-1- 9% (Diastereomer1) | LC-MS (1) 3.46 ESI+: 487 (M + H)⁺ |
| 23A | 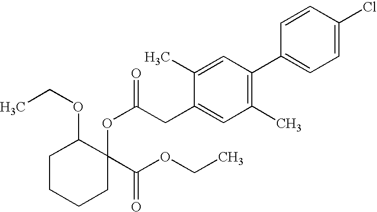 | GWP 2007-06-15-1- 13% | LC-MS (2) 3.21 ESI+: 473 (M + H)⁺ |
| 24A | 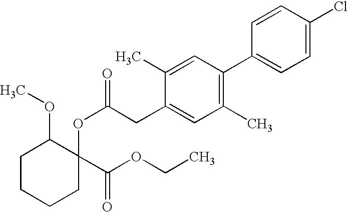 | GWP 2007-06-15-1- 32% | LC-MS (1) 3.32 ESI+: 459 (M + H)⁺ |
| 25A | 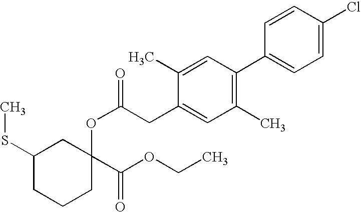 | GWP 2007-06-15-1- 26% | LC-MS (2) 3.26 ESI+: 475 (M + H)⁺ |
| 26A | 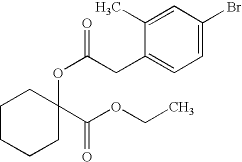 | GWP 2007-06-15-1- 29% | LC-MS (3) 3.19 ESI+: 383 (M + H)⁺ |
| 27A | 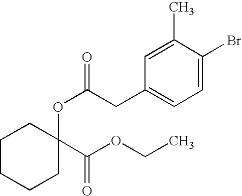 | GWP 2007-06-15-1- 59% | LC-MS (2) 2.99 ESI+: 383 (M + H)⁺ |
| 28A | 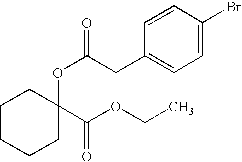 | GWP 2007-06-15-1- 72% | LC-MS (3) 3.09 ESI+: 369 (M + H)⁺ |
| 29A | 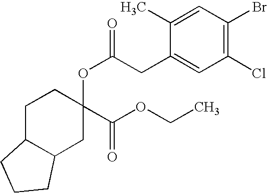 | GWP 2007-06-15-1- 49% | LC-MS (1) 3.42 |
| 30A | 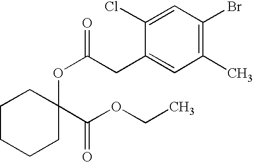 | GWP 2007-06-15-1- 60% | LC-MS (1) 3.51 ESI+: 443 (M + H)⁺ |
| 31A | 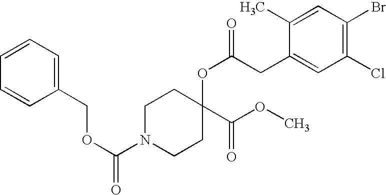 | GWP 2007-06-15-1- 18% | LC-MS (1) 3.05 ESI+: 538 (M + H)⁺ |
| 32A | 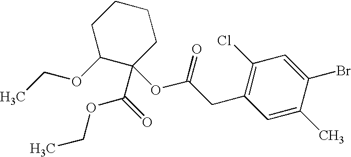 | GWP2 42% | LC-MS (1) 3.35 ESI+: 460 (M + H)⁺ |
| 33A | 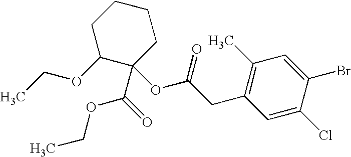 | GWP2 81% | LC-MS (2) 2.92 ESI+: 463 (M + H)⁺ |
| 34A | 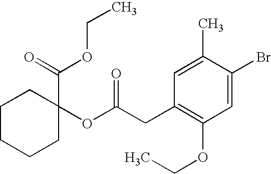 | GWP1 (10 eq. SOCl₂, 4 eq. DIEA 7% | LC-MS (1) 3.18 MS (DCI): 444 (M + NH₄)⁺ |

### Example 35A

### 1-Benzyl-3-methyl-3-{[(4-bromo-5-chloro-2-methylphenyl)acetyl]oxy}piperidine-1,3-dicarboxylate

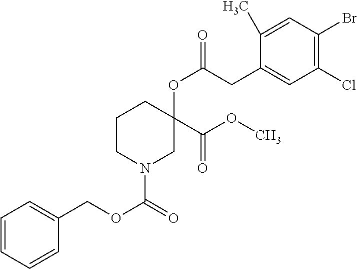

13.1 g (56.23 mmol) of thiocarbonic acid-O,O-di-(2-pyridyl ester), 0.624 g (5.11 mmol) of 4-dimethylaminopyridine and 15.3 g (51.12 mmol) of 1-benzyl-3-methyl-3-hydroxypiperidine-1,3-dicarboxylate from example 4A are added to a solution of 14.8 g (56.23 mmol) of 4-bromo-5-chloro-2-methylphenyl)acetic acid in 250 ml of toluene and the mixture is stirred for 12 hours at 80° C. After cooling the mixture is concentrated on a rotary evaporator and the residue obtained is separated by preparative HPLC. 5.8 g (20% th.) of product are obtained. LC-MS (Method 2): Rₜ=2.96 min. MS (ESIpos): m/z=538 (M+H)⁺. ¹H NMR (300 MHz, DMSO-d₆): δ=7.34 (d, 7H), 5.14-5.02 (m, 2H), 4.42-4.28 (m, 1H), 3.94 (d, 1H), 3.69-3.51 (m, 5H), 3.45-3.23 (m, 1H, masked by water), 3.07-2.85 (m, 1H), 2.15 (s, 3H), 2.07-1.78 (m, 2H), 1.65-1.51 (m, 2H).

### Example 36A

### 3-(4-Bromo-5-chloro-2-methylphenyl)-4-hydroxy-1-oxaspiro[4.5]dec-3-en-2-one

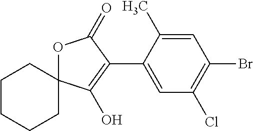

1.29 g (11.5 mmol) of potassium-tert.-butylate are provided in 30 ml of DMF under argon at 0° C., a solution of 3.20 g (7.66 mmol) of 1-ethoxycarbonylcyclohexyl 4-bromo-5-chloro-2-methylphenylacetate (example 6A) in 30 ml of DMF is added dropwise and the mixture is stirred overnight at RT. The reaction mixture is subsequently poured into an ice-cold 1N aqueous hydrochloride solution, and the precipitate is collected by suction filtration, washed with water and dried. 2.73 g (96% th.) of product are obtained. LC-MS (Method 1): Rₜ=2.53 min. MS (ESIpos): m/z=371 (M+H)⁺. ¹H NMR (300 MHz, DMSO-d₆): δ=12.4 (s, 1H), 7.68 (s, 1H), 7.36 (s, 1H), 2.13 (s, 3H), 1.89 (dt, 2H), 1.78-1.67 (m, 3H), 1.66-1.52 (m, 4H), 1.34-1.16 (m, 1H).

GWP3: Dieckmann Condensation

Potassium tert.-butylate (1.5 eq) is provided in DMF at 0° C. under argon, a solution of the phenylacetic acid ester in DMF is added dropwise and the reaction mixture is stirred overnight at RT. The reaction mixture is subsequently poured into an ice-cold 1N aqueous hydrochloride solution, the precipitate is collected by suction filtration, washed with water and dried. Purification or where appropriate separation of the diastereomers is carried out by preparative HPLC (RP18 column; eluent: acetonitrile-water gradient, 0.1% formic acid). Alternatively the purification or separation of the diastereomers can take place by column chromatography on silica gel 60 (eluent: cyclohexane/ethyl acetate gradient) or flash chromatography (eluent: cyclohexane/ethyl acetate gradient).

If no precipitate forms on addition onto the ice-cold 1N aqueous hydrochloride solution the aqueous solution may alternatively be extracted with ethyl acetate. The combined organic phases are dried over sodium sulfate, filtered, concentrated and purified as described.

The following compounds are prepared in analogy to example 36A, GWP 3 and the general preparative information. Some of the products are obtained after chromatographic separation of the diastereomeric or enantiomeric mixtures.

| | | | Analysis |
|---|---|---|---|
| | | Prepared from | LC-MS (Method) |
| Ex. | | according to GWP | Rₜ [min] |
| No. | Structure | Yield [th.] | MS: m/z |
| 37A | 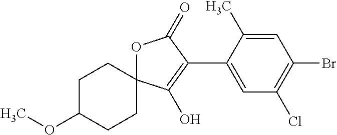 | Example 8A GWP3 35% (Diastereomer 1) | LC-MS (1) 2.27 ESI+: 401 (M + H)⁺ |
| 38A | 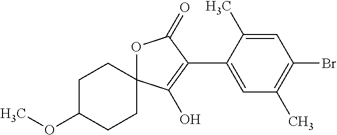 | Example 9A GWP3 28% (Diastereomer 1) | LC-MS (3) 2.26 ESI+: 381 (M + H)⁺ |
| 39A | 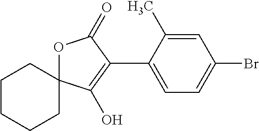 | Example 26A GWP3 85% | LC-MS (1) 2.40 ESI+: 337 (M + H)⁺ |
| 40A | 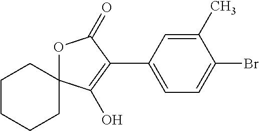 | Example 27A GWP3 96% | LC-MS (3) 2.54 ESI+: 337 (M + H)⁺ |
| 41A | 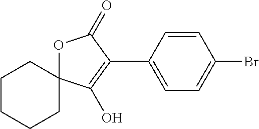 | Example 28A GWP3 94% | LC-MS (1) 2.36 ESI+: 323 (M + H)⁺ |
| 42A | 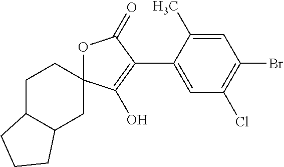 | Example 29A GWP3 97% | LC-MS (1) 2.86 ESI+: 411 (M + H)⁺ |
| 43A | 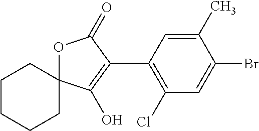 | Example 30A GWP3 89% | LC-MS (1) 2.50 ESI+: 371 (M + H)⁺ |
| 44A | 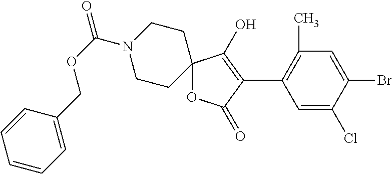 | Example 31A GWP3 98% | LC-MS (2) 2.42 ESI+: 506 (M + H)⁺ |
| 45A | 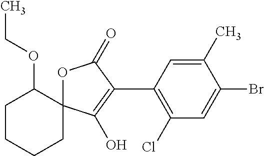 | Example 32A GWP3 18% | LC-MS (2) 2.30 ESI+: 415 (M + H)⁺ |
| 46A | 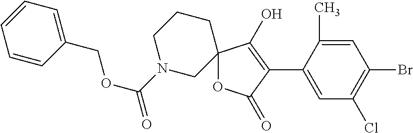 | Example 34A GWP3 100% | LC-MS (3) 2.64 ESI+: 506 (M + H)⁺ |
| 47A | 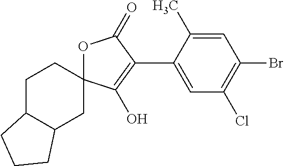 | Example 29A Stereoisomer 1 26% | LC-MS (2) 2.65 ESI+: 411 (M + H)⁺ |
| 48A | 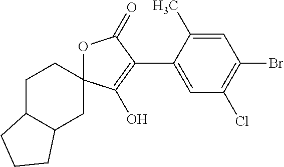 | Example 29A Stereoisomer 2 27% | LC-MS (1) 2.87 ESI+: 411 (M + H)⁺ |
| 49A | 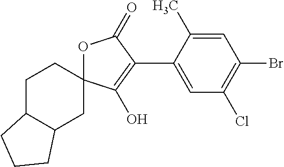 | Example 29A Stereoisomer 3 10% | LC-MS (1) 2.86 ESI+: 411 (M + H)⁺ |
| 50A | 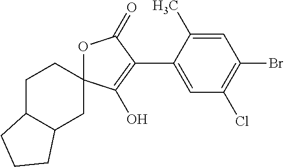 | Example 29A Stereoisomer 4 11% | LC-MS (1) 2.86 ESI+: 411 (M + H)⁺ |
| 51A | 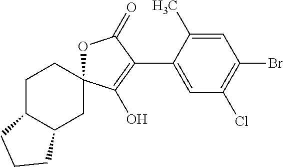 | Example 29A 11% | LC-MS (3) 2.94 ESI+: 411 (M + H)⁺ |
| 52A | 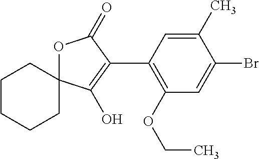 | Example 34A GWP3 21% | LC-MS (1) 2.81 ESI+: 381 (M + H)⁺ |

The following compound is prepared in analogy to Example 6A, the respective GWP and the general preparative information:

| | | | Analysis |
|---|---|---|---|
| | | Prepared | LC-MS |
| | | according | (Method) |
| Ex. | | to GWP | Rₜ [min] |
| No. | Structure | Yield [th.] | MS: m/z |
| 53A | 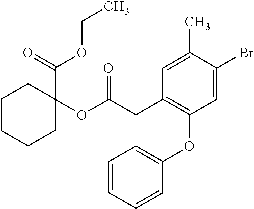 | GWP2 76% | LC-MS (2) 3.23 ESI+: 444 (M + H)⁺ |

The following compound is prepared in analogy to example 36A, GWP 3 and the general preparative information.

| | | Prepared | Analysis |
|---|---|---|---|
| | | from | LC-MS |
| | | according | (Method) |
| Ex. | | to GWP | Rₜ [min] |
| No. | Structure | yield [th.] | MS: m/z |
| 54A | 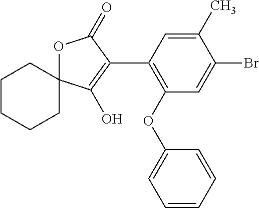 | Example 34A GWP3 48% | LC-MS (1) 2.52 ESI+: 448 (M + H)⁺ |

### Example 55A

### Ethyl 1-[2-(4-bromo-5-chloro-2-methylphenyl)acetoxy]cycloheptanoate

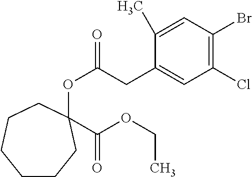

1.388 g (5.86 mmol) of thiocarbonic acid O,O-di-(2-pyridyl ester), 1.0 g (5.37 mmol) of ethyl 1-hydroxycycloheptanoate and 60 mg (0.49 mmol) of DMAP are added to a solution of 1.326 g (4.88 mmol) of (4-bromo-5-chloro-2-methylphenyl)acetic acid in 25 ml of MTBE and the mixture is boiled overnight at reflux. After cooling the precipitate is filtered off and the filtrate is evaporated in vacuum (2.5 g). After silica gel chromatography using iso-hexane/ethyl acetate 20:1 1.29 g (45% th.) of an oil are obtained. LC-MS (Method 5): Rₜ=4.80 min. MS (ESIpos): m/z=507 (M+77)⁺.

### Prepared in Analogy to the Method for Example 55A

| | | | Analysis |
|---|---|---|---|
| | | | LC-MS |
| | | | (Method) |
| Ex. | | | Rₜ [min] |
| No. | Structure | Yield [th.] | MS: m/z |
| 56A | 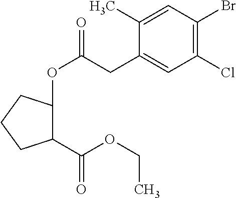 | Reacted further as crude product | LC-MS (6) 3.19 ESI+: 479 (M + 77)⁺ |

The following compounds are prepared in analogy to Example 36A, GWP3 and the general preparative information.

| | | Prepared | Analysis |
|---|---|---|---|
| | | from | LC-MS |
| | | according | (Method) |
| Ex. | | to GWP | Rₜ [min] |
| No. | Structure | Yield [th.] | MS: m/z |
| 57A | 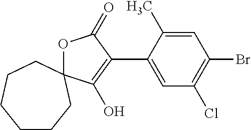 | Example 55A GWP3, (4 eq. KOtBu) 34% | LC-MS (6) 2.97 ESI+: 385 (M + H)⁺ |
| 58A | 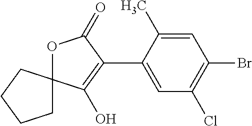 | Example 56A GWP3, (4 eq. KOtBu) 54% | LC-MS (7) 3.39 ESI+: 357 (M + H)⁺ |

### Exemplary Embodiments

The following compounds are prepared in analogy to Example 36A, GWP3 and the general preparative information.

| | | | Analysis |
|---|---|---|---|
| | | Prepared from | LC-MS (Method) |
| Ex. | | according to GWP | Rₜ [min] |
| No. | Structure | Yield [th.] | MS: m/z |
| 1 | 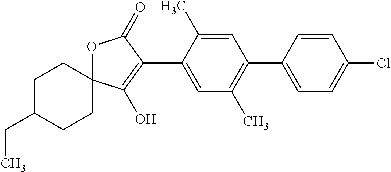 | Example 10A GWP3 80% | LC-MS (1) 3.12 ESI+: 411 (M + H)⁺ |
| 2 | 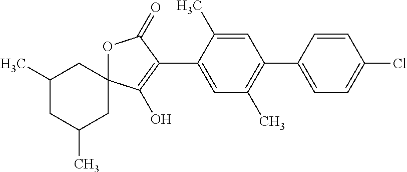 | Example 11A GWP3 31% (Diastereomer 1) | LC-MS (3) 3.11 ESI+: 411 (M + H)⁺ |
| 3 | 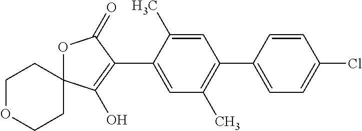 | Example 12A GWP3 80% | LC-MS (3) 2.58 ESI+: 385 (M + H)⁺ |
| 4 | 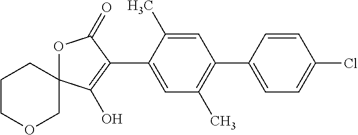 | Example 13A GWP3 79% | LC-MS (3) 2.54 ESI+: 385 (M + H)⁺ |
| 5 | 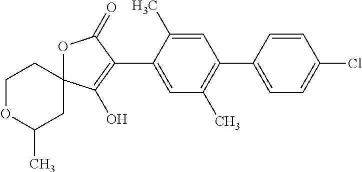 | Example 14A GWP3 76% | LC-MS (3) 2.66 ESI+: 399 (M + H)⁺ |
| 6 | 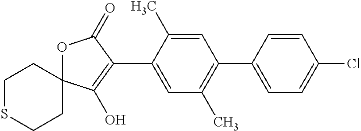 | Example 15A GWP3 80% | LC-MS (3) 2.79 ESI+: 401 (M + H)⁺ |
| 7 | 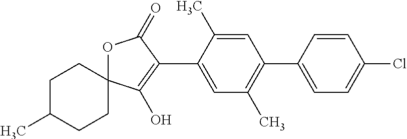 | Example 16A GWP3 88% | LC-MS (3) 3.04 ESI+: 397 (M + H)⁺ |
| 8 | 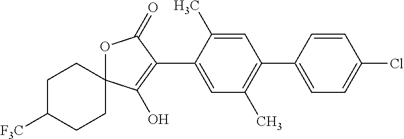 | Example 17A GWP3 94% | LC-MS (3) 3.01 ESI+: 451 (M + H)⁺ |
| 9 | 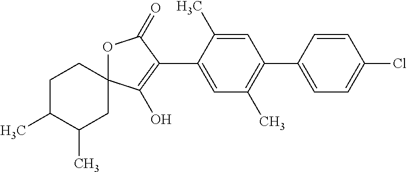 | Example 18A GWP3 90% | LC-MS (3) 3.12 ESI+: 411 (M + H)⁺ |
| 10 | 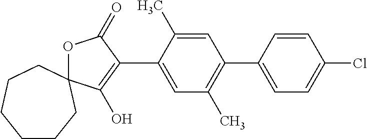 | Example 19A GWP3 83% | LC-MS (3) 3.02 ESI+: 397 (M + H)⁺ |
| 11 | 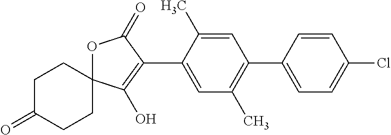 | Example 20A GWP3 57% | LC-MS (2) 2.27 ESI+: 397 (M + H)⁺ |
| 12 | 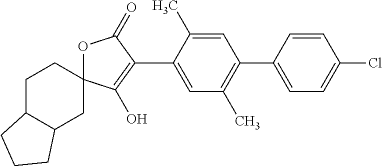 | Example 21A GWP3 83% | LC-MS (1) 3.10 ESI+: 423 (M + H)⁺ |
| 13 | 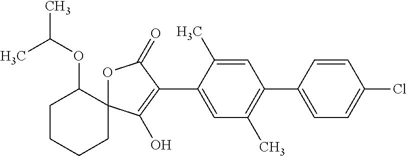 | Example 22A GWP3 66% | LC-MS (3) 2.95 ESI+: 441 (M + H)⁺ |
| 14 | 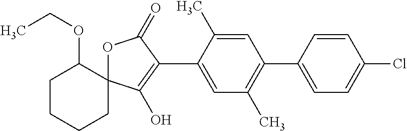 | Example 23A GWP3 92% | LC-MS (1) 2.84 ESI+: 427 (M + H)⁺ |
| 15 | 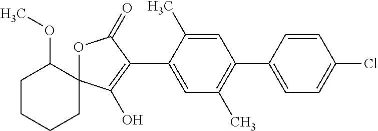 | Example 24A GWP3 28% | LC-MS (1) 2.72 ESI+: 413 (M + H)⁺ |
| 16 | 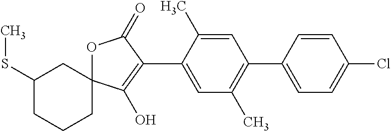 | Example 25A GWP3 93% | LC-MS (3) 2.92 ESI+: 429 (M + H)⁺ |

### Example 17

### 3-(2-Chloro-5-methylbiphenyl-4-yl)-4-hydroxy-1-oxaspiro[4.5]dec-3-en-2-one

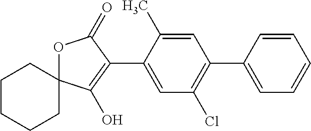

100 mg (0.27 mmol) of 3-(4-bromo-5-chloro-2-methylphenyl)-4-hydroxy-1-oxaspiro[4.5]dec-3-en-2-one (example 36A), 36.1 mg (0.30 mmol) of phenylboronic acid, 1.8 mg (0.01 mmol) of palladium(II) acetate, 9.0 mg (0.02 mmol) of dicyclohexyl-(2′,4′,6′-triisopropylbiphenyl-2-yl)phosphine and 263 mg (0.81 mmol) of cesium carbonate are mixed. The mixture is degassed and vented twice with argon, 1 ml of DME is added, the mixture is degassed and vented twice with argon and heated overnight at 50° C. After cooling the reaction mixture is poured into a 1N aqueous hydrochloride solution, the aqueous phase is extracted with DCM, and the combined organic phases are dried over sodium sulfate, filtered and concentrated. After preparative HPLC (RP18 column; eluent: acetonitrile-water gradient, 0.1% formic acid) 62 mg (63% th.) of product are obtained. LC-MS (Method 2): Rₜ=2.49 min. MS (ESIpos): m/z=369 (M+H)⁺. ¹H NMR (400 MHz, DMSO-d₆): δ=12.4 (s, 1H), 7.52-7.38 (m, 5H), 7.31 (s, 2H), 2.18 (s, 3H), 1.92 (dt, 2H), 1.79-1.68 (m, 3H), 1.67-1.52 (m, 4H), 1.34-1.19 (m, 1H).

GWP4: Suzuki Coupling (1)

The aryl halide (1.0 eq), the boronic acid (1.1 eq), the catalyst palladium (II) acetate (0.03 eq), the ligand dicyclohexyl-(2′,4′,6′-triisopropylbiphenyl-2-yl)phosphine (0.07 eq) and the base cesium carbonate (3 eq) are mixed. The mixture is degassed and vented twice with argon, DME is added, the mixture is degassed and vented twice with argon and heated overnight at 50° C. After cooling the reaction mixture is poured into a 1N aqueous hydrochloride solution, the aqueous phase is extracted with DCM, and the combined organic phases are dried over sodium sulfate, filtered and concentrated. Purification is carried out by preparative HPLC (RP18 column; eluent: acetonitrile-water gradient, 0.1% formic acid). Alternatively the purification can take place by column chromatography on silica gel 60 (eluent: cyclohexane/ethyl acetate gradient) or flash chromatography (eluent: cyclohexane/ethyl acetate gradient).

GWP5: Suzuki Coupling (2)

The aryl halide (1.0 eq), the boronic acid (1.1 eq) and DME are mixed and degassed and vented with argon three times. The catalyst tetrakis(triphenylphosphine)palladium(0) (0.06 eq) and a degassed 20% aqueous sodium carbonate solution (10 eq) are added and the mixture is heated overnight at 80° C. After cooling the reaction mixture is poured into 1N aqueous hydrochloric acid, the aqueous phase is extracted with DCM, and the combined organic phases are dried over sodium sulfate, filtered and concentrated. Purification is carried out by preparative HPLC (RP18 column; eluent: acetonitrile-water gradient, 0.1% formic acid). Alternatively the purification can take place by column chromatography on silica gel 60 (eluent: cyclohexane/ethyl acetate gradient) or flash chromatography (eluent: cyclohexane/ethyl acetate gradient).

Alternatively a mixture of toluene and ethanol can also be used as solvent and the mixture can be heated under reflux.

GWP6: Suzuki Coupling (3)

The aryl halide (1.0 eq) and the boronic acid (1.1 eq) are mixed in DME, water and ethanol (3:2:1), degassed and vented with argon three times. The catalyst tetrakis(triphenylphosphine)palladium(0) (0.04 eq) and cesium carbonate (3 eq.) are added and the mixture is heated overnight at 50° C. After cooling the reaction mixture is poured into 1 molar aqueous hydrochloric acid, the aqueous phase is extracted with DCM, and the combined organic phases are dried over sodium sulfate, filtered and concentrated. Purification is carried out with preparative HPLC (RP18 column; eluent: acetonitrile-water gradient, 0.1% formic acid). Alternatively purification can be carried out by column chromatography on silica gel 60 (eluent: cyclohexane/ethyl acetate gradient) or flash chromatography (eluent: cyclohexane/ethyl acetate gradient).

The following compounds are prepared in analogy to example 17, the respective GWP and the general preparative information:

| | | | Analysis |
|---|---|---|---|
| | | Prepared from | LC-MS (Method) |
| Ex. | | by GWP | Rₜ [min] |
| No. | Structure | Yield [th.] | MS: m/z |
| 18 | 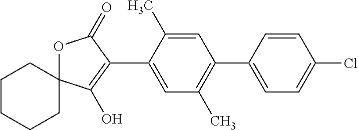 | Example 36A GWP5 37% | LC-MS (2) 2.68 ESI+: 403 (M + H)⁺ |
| 19 | 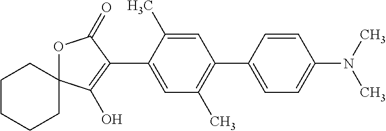 | Example 36A GWP4 20% | LC-MS (1) 2.55 ESI+: 412 (M + H)⁺ |
| 20 | 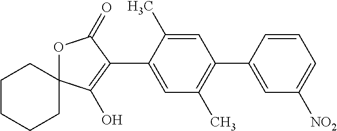 | Example 36A GWP4 28% | LC-MS (3) 2.74 ESI+: 414 (M + H)⁺ |
| 21 | 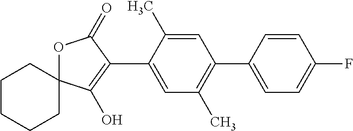 | Example 36A GWP4 47% | LC-MS (2) 2.53 ESI+: 387 (M + H)⁺ |
| 22 | 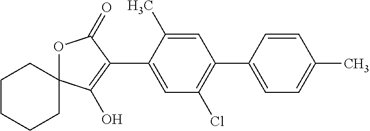 | Example 36A GWP4 50% | LC-MS (3) 2.85 ESI+: 383 (M + H)⁺ |
| 23 | 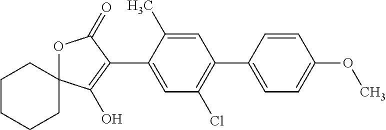 | Example 36A GWP4 47% | LC-MS (2) 2.49 ESI+: 399 (M + H)⁺ |
| 24 | 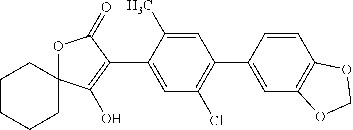 | Example 36A GWP4 50% | LC-MS (2) 2.45 ESI+: 413 (M + H)⁺ |
| 25 | 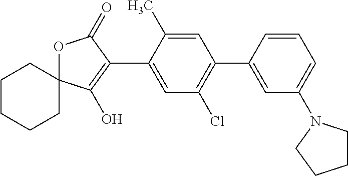 | Example 36A GWP4 22% | LC-MS (2) 1.52 ESI+: 438 (M + H)⁺ |
| 26 | 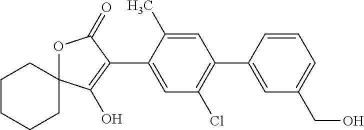 | Example 36A GWP4 23% | LC-MS (2) 2.12 ESI+: 399 (M + H)⁺ |
| 27 | 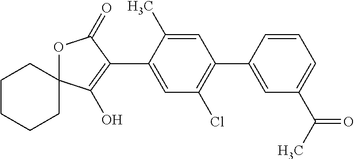 | Example 36A GWP4 49% | LC-MS (1) 2.60 ESI+: 411 (M + H)⁺ |
| 28 | 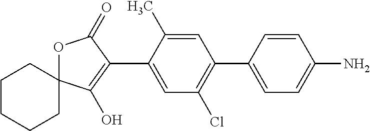 | Example 36A GWP4 20% | LC-MS (1) 2.28 ESI+: 384 (M + H)⁺ |
| 29 | 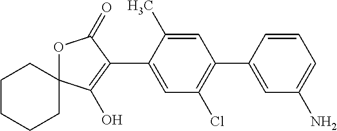 | Example 36A GWP4 6% | LC-MS (3) 2.69 ESI+: 384 (M + H)⁺ |
| 30 | 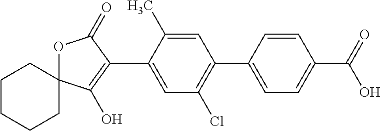 | Example 36A GWP5 49% | LC-MS (1) 2.36 ESI+: 413 (M + H)⁺ |
| 31 | 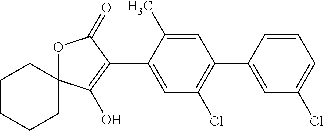 | Example 36A GWP4 16% | LC-MS (3) 2.55 ESI+: 403 (M + H)⁺ |
| 32 | 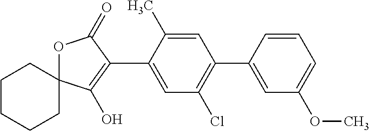 | Example 36A GWP4 44% | LC-MS (1) 2.74 ESI+: 399 (M + H)⁺ |
| 33 | 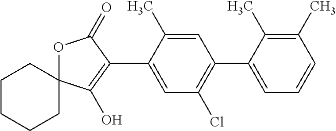 | Example 36A GWP4 63% | LC-MS (3) 2.92 ESI+: 397 (M + H)⁺ |
| 34 | 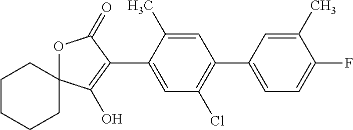 | Example 36A GWP4 66% | LC-MS (3) 2.89 ESI+: 401 (M + H)⁺ |
| 35 | 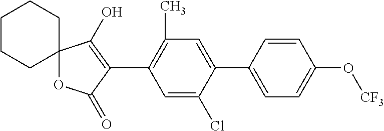 | Example 36A GWP4 41% | LC-MS (2) 2.78 ESI+: 453 (M + H)⁺ |
| 36 | 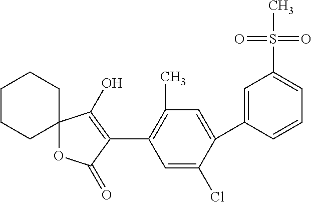 | Example 36A GWP4 71% | LC-MS (2) 2.21 ESI+: 447 (M + H)⁺ |
| 37 | 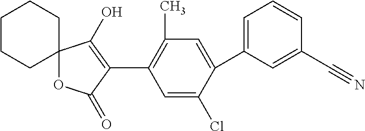 | Example 36A GWP4 21% | LC-MS (1) 2.48 ESI+: 394 (M + H)⁺ |
| 38 | 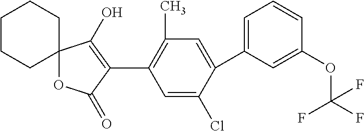 | Example 36A GWP4 29% | LC-MS (2) 2.78 ESI+: 453 (M + H)⁺ |
| 39 | 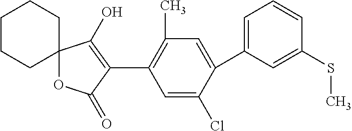 | Example 36A GWP4 18% | LC-MS (3) 2.87 ESI+: 415 (M + H)⁺ |
| 40 | 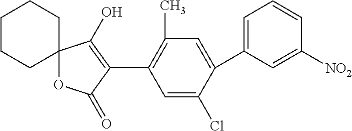 | Example 36A GWP4 70% | LC-MS (1) 2.66 ESI+: 414 (M + H)⁺ |
| 41 | 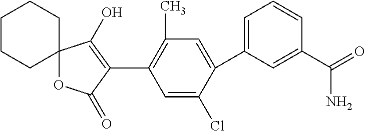 | Example 36A GWP4 70% | LC-MS (2) 2.02 ESI+: 412 (M + H)⁺ |
| 42 | 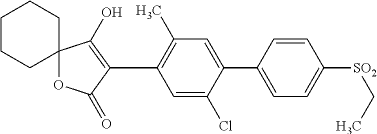 | Example 36A GWP4 27% | LC-MS (1) 2.45 ESI+: 461 (M + H)⁺ |
| 43 | 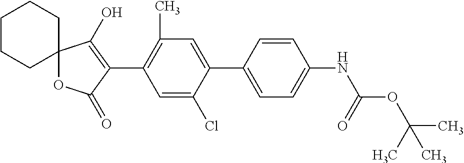 | Example 36A GWP4 22% | LC-MS (3) 2.0 ESI+: 484 (M + H)⁺ |
| 44 | 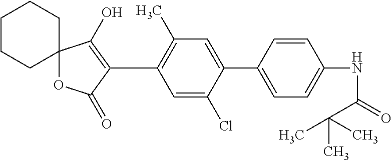 | Example 36A GWP4 17% | LC-MS (3) 2.73 ESI+: 468 (M + H)⁺ |
| 45 | 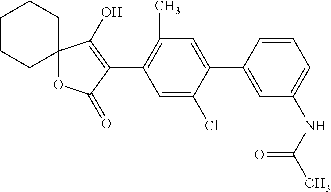 | Example 36A GWP4 26% | LC-MS (1) 2.34 ESI+: 426 (M + H)⁺ |
| 46 | 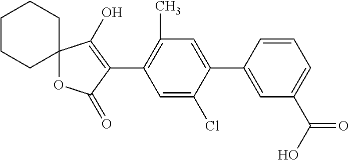 | Example 36A GWP4 61% | LC-MS (3) 2.43 ESI+: 413 (M + H)⁺ |
| 47 | 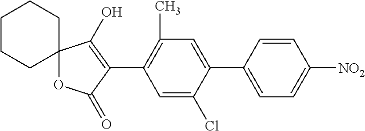 | Example 36A GWP4 52% | LC-MS (2) 2.52 ESI+: 414 (M + H)⁺ |
| 48 | 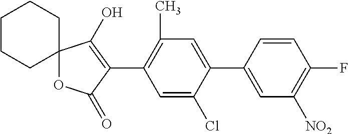 | Example 36A GWP4 8% | LC-MS (1) 2.51 ESI+: 432 (M + H)⁺ |
| 49 | 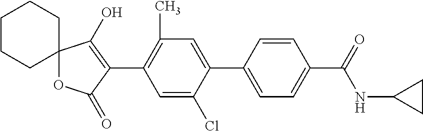 | Example 36A GWP6 21% | LC-MS (1) 2.37 ESI+: 452 (M + H)⁺ |
| 50 | 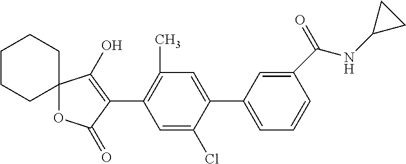 | Example 36A GWP6 39% | LC-MS (1) 2.4 ESI+: 452 (M + H)⁺ |
| 51 | 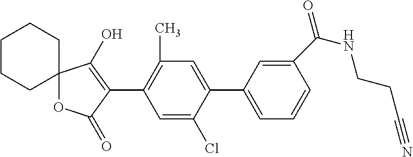 | Example 36A GWP6 23% | LC-MS (1) 2.31 ESI+: 465 (M + H)⁺ |
| 52 | 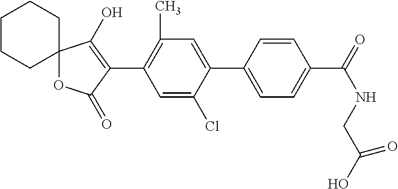 | Example 36A GWP6 44% | LC-MS (1) 2.13 ESI+: 470 (M + H)⁺ |
| 53 | 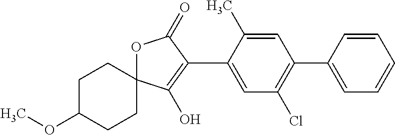 | Example 37A GWP4 26% | LC-MS (1) 2.47 ESI+: 399 (M + H)⁺ |
| 54 | 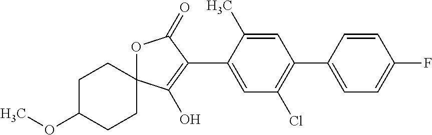 | Example 37A GWP4 28% | LC-MS (1) 2.50 ESI+: 417 (M + H)⁺ |
| 55 | 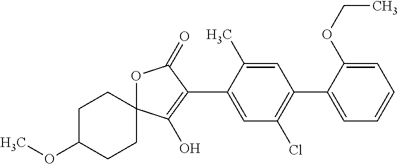 | Example 37A GWP4 38% | LC-MS (2) 2.34 ESI+: 443 (M + H)⁺ |
| 56 | 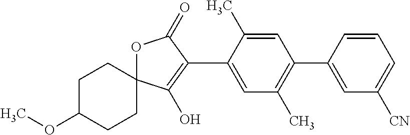 | Example 38A GWP4 6% | LC-MS (1) 2.34 ESI+: 404 (M + H)⁺ |
| 57 | 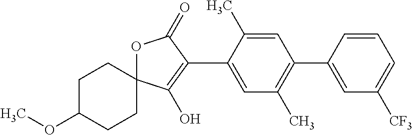 | Example 38A GWP4 18% | LC-MS (3) 2.71 ESI+: 447 (M + H)⁺ |
| 58 | 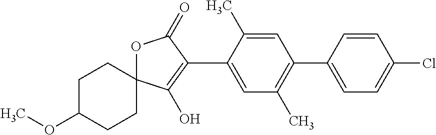 | Example 38A Epimer separation | LC-MS (2) 2.49 ESI+: 413 (M + H)⁺ |
| 59 | 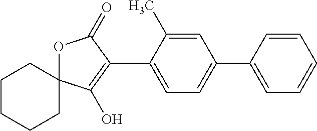 | Example 39A GWP4 44% | LC-MS (1) 2.63 ESI+: 335 (M + H)⁺ |
| 60 | 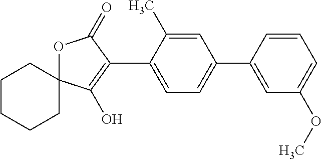 | Example 39A GWP4 47% | LC-MS (1) 2.62 ESI+: 365 (M + H)⁺ |
| 61 | 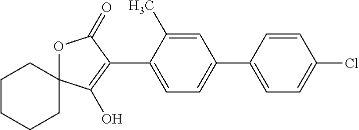 | Example 39A GWP4 49% | LC-MS (2) 2.56 ESI+: 369 (M + H)⁺ |
| 62 | 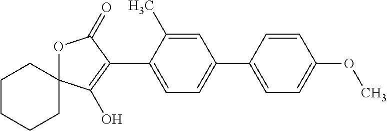 | Example 39A GWP4 22% | LC-MS (3) 2.61 ESI+: 365 (M + H)⁺ |
| 63 | 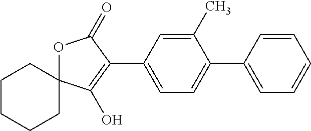 | Example 40A GWP4 19% | LC-MS (1) 2.67 ESI+: 335 (M + H)⁺ |
| 64 | 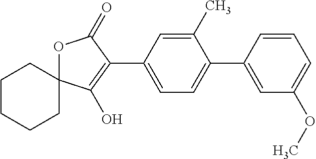 | Example 40A GWP4 44% | LC-MS (1) 2.65 ESI+: 365 (M + H)⁺ |
| 65 | 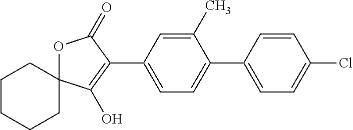 | Example 40A GWP4 35% | LC-MS (1) 2.85 ESI+: 369 (M + H)⁺ |
| 66 | 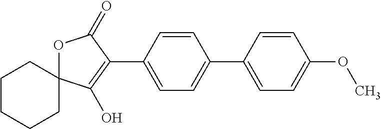 | Example 41A GWP4 39% | LC-MS (1) 2.56 ESI+: 351 (M + H)⁺ |
| 67 | 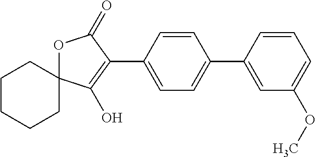 | Example 41A GWP4 63% | LC-MS (1) 2.57 ESI+: 351 (M + H)⁺ |
| 68 | 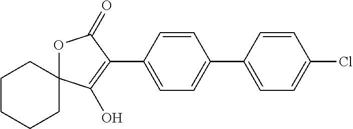 | Example 41A GWP4 24% | LC-MS (2) 2.52 ESI+: 355 (M + H)⁺ |
| 69 | 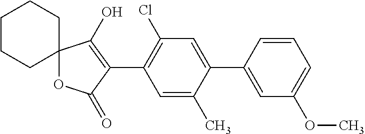 | Example 43A GWP4 30% | LC-MS (1) 2.71 ESI+: 399 (M + H)⁺ |
| 70 | 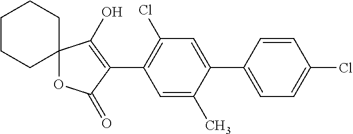 | Example 43A GWP4 38% | LC-MS (3) 2.90 ESI+: 403 (M + H)⁺ |
| 71 | 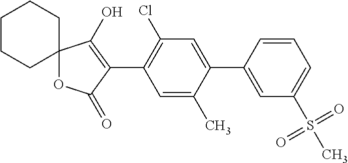 | Example 43A GWP4 21% | LC-MS (3) 2.42 ESI+: 447 (M + H)⁺ |
| 72 | 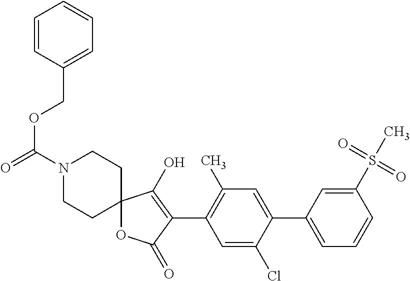 | Example 44A GWP 40% | LC-MS (3) 2.50 ESI+: 582 (M + H)⁺ |
| 73 | 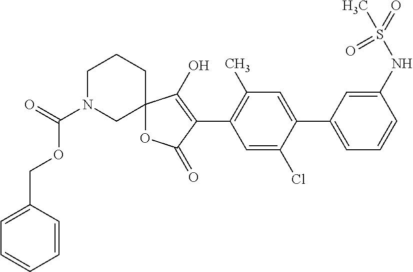 | Example 46A GWP4 25% | LC-MS (3) 2.55 ESI+: 597 (M + H)⁺ |
| 74 | 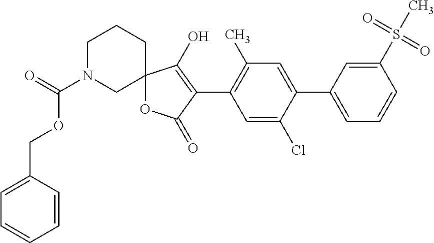 | Example 46A GWP4 69% | LC-MS (3) 2.55 ESI+: 582 (M + H)⁺ |
| 75 | 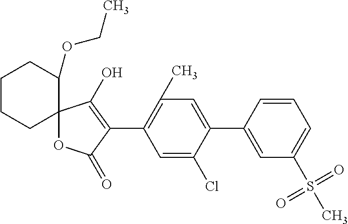 | Example 33A GWP6/GWP3 Diastereomer 1 74% | LC-MS (2) 2.15 ESI+: 491 (M + H)⁺ |
| 76 | 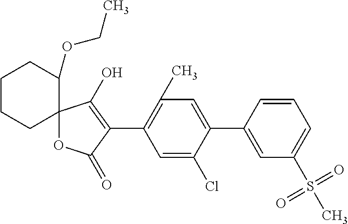 | Example 33A GWP6/GWP3 Diastereomer 2 66% | LC-MS (3) 2.59 ESI+: 491 (M + H)⁺ |
| 77 | 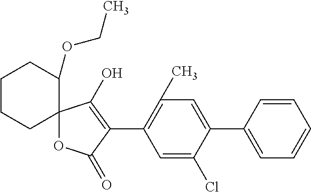 | Example 33A GWP6/GWP3 Diastereomer 1 92% | LC-MS (2) 2.48 ESI+: 413 (M + H)⁺ |
| 78 | 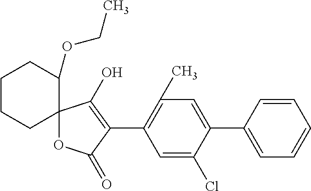 | Example 33A GWP6/GWP3 Diastereomer 2 65% | LC-MS (3) 2.94 ESI+: 413 (M + H)⁺ |
| 79 | 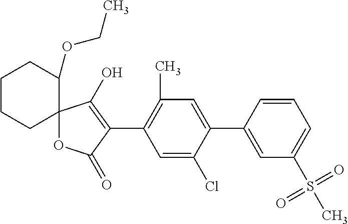 | Example 33A Enantiomer 2 34% | LC-MS (1) 2.38 ESI+: 491 (M + H)⁺ |
| 80 | 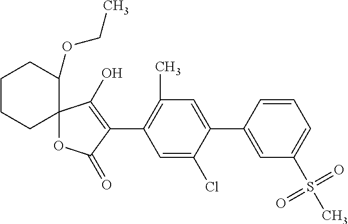 | Example 33A Enantiomer 1 35% | LC-MS (1) 491 ESI+: (M + H)⁺ |
| 81 | 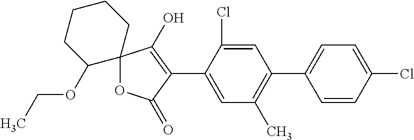 | Example 45A GWP4 Diastereomer 1 49% | LC-MS (3) 2.89 ESI+: 447 (M + H)⁺ |
| 82 | 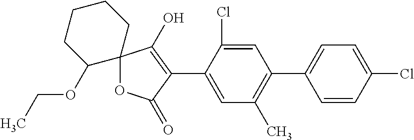 | Example 45A Diastereomer 2 100% | LC-MS (3) 2.89 ESI+: 447 (M + H)⁺ |
| 83 | 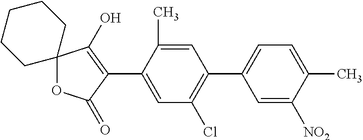 | Example 36A GWP4 40% | LC-MS (3) 2.83 ESI+: 428 (M + H)⁺ |
| 84 | 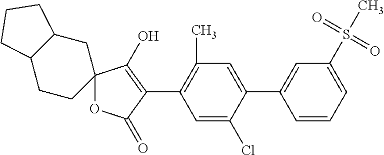 | Example 48A GWP4 51% | LC-MS (3) 2.79 ESI+: 487 (M + H)⁺ |
| 85 | 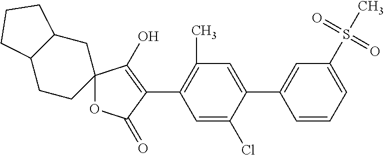 | Example 47A GWP4 79% | LC-MS (1) 2.72 ESI+: 487 (M + H)⁺ |
| 86 | 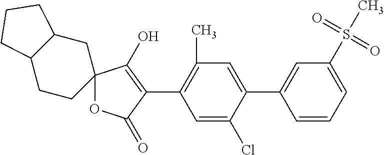 | Example 49A GWP4 70% | LC-MS (1) 2.70 ESI+: 487 (M + H)⁺ |
| 87 | 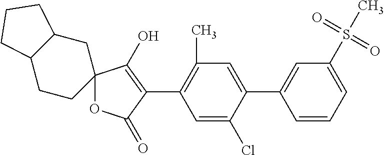 | Example 50A GWP4 47% | LC-MS (1) 2.70 ESI+: 487 (M + H)⁺ |
| 88 | 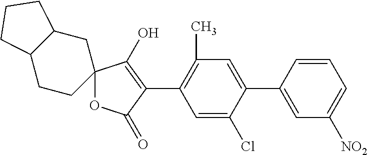 | Example 48A GWP4 79% | LC-MS (1) 2.87 ESI+: 454 (M + H)⁺ |
| 89 | 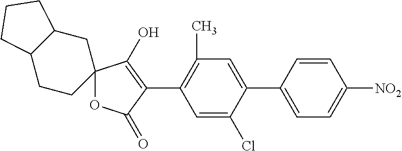 | Example 48A GWP4 73% | LC-MS (1) 2.87 ESI+: 454 (M + H)⁺ |
| 90 | 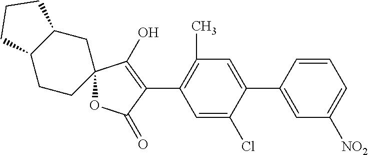 | Example 51A GWP4 59% | LC-MS (1) 3.01 ESI+: 454 (M + H)⁺ |
| 91 | 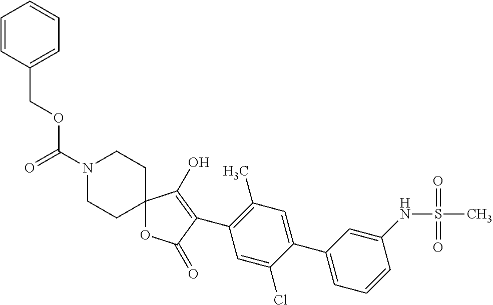 | Example 44A GWP6 58% | LC-MS (2) 2.27 ESI+: 597 (M + H)⁺ |
| 92 | 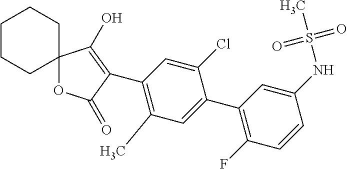 | Example 36A GWP6 14% | LC-MS (2) 2.21 ESI+: 480 (M + H)⁺ |
| 93 | 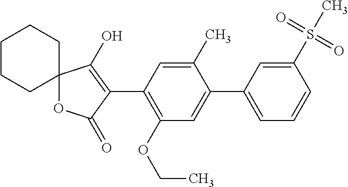 | Example 52A GWP6 83% | LC-MS (1) 2.64 ESI+: 457 (M + H)⁺ |

### Example 94

### N-[2′-Chloro-4′-(4-hydroxy-2-oxo-1-oxa-7-azaspiro[4.5]dec-3-en-3-yl)-5′-methylbiphenyl-3-yl]methanesulfonamide trifluoroacetate

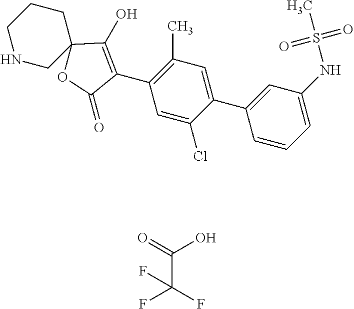

A solution of 44 mg (0.07 mmol) of benzyl 3-{2-chloro-5-methyl-3′-[(methylsulfonyl)amino]biphenyl-4-yl}-4-hydroxy-2-oxo-1-oxa-7-azaspiro[4.5] dec-3-ene-7-carboxylate from example 73 in 3 ml of trifluoroacetic acid is stirred for 12 hours at room temperature. The reaction solution is concentrated on a rotary evaporator and reacted further without purification. 50 mg (0.09 mmol, 83% th.) of product are obtained. LC-MS (Method 1): Rₜ=1.50 min. MS (ESIpos): m/z=463 (M+H)⁺

### Example 95

### 3-[2-Chloro-5-methyl-3′-(methylsulfonyl)biphenyl-4-yl]-4-hydroxy-1-oxa-7-azaspiro[4.5]dec-3-en-2-one hydrochloride

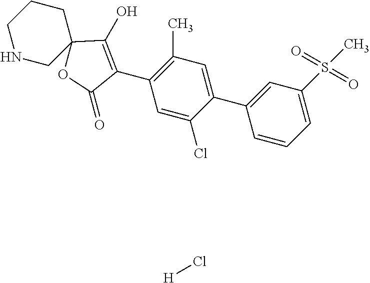

A solution of 1.4 g (2.41 mmol) of benzyl 3-[2-chloro-5-methyl-3′-(methylsulfonyl)biphenyl-4-yl]-4-hydroxy-2-oxo-1-oxa-7-azaspiro[4.5]dec-3-ene-7-carboxylate from example 74 in 15 ml of trifluoroacetic acid is stirred for 12 hours at room temperature. The reaction solution is concentrated on a rotary evaporator and the residue obtained is separated by preparative HPLC (eluent: acetonitrile/water+1 vol % 1N hydrochloric acid). 938 mg (1.9 mmol, 80% th.) of product are obtained. LC-MS (Method 1): Rₜ=2.93 min. MS (ESIpos): m/z=448 (M+H)⁺. ¹H NMR (400 MHz, DMSO-d₆): δ=9.84-9.67 (m, 1H), 9.0-8.78 (m, 1H), 8.05-7.94 (m, 2H), 7.9-7.72 (m, 2H), 7.44 (d, 2H), 3.66-3.53 (m, 1H), 3.42 (d, 1H), 3.26-3.2 (m, 1H, masked by water), 3.0-2.86 (m, 1H), 2.5 (s, 3H), 2.4-2.27 (m, 1H), 2.24 (s, 3H), 2.0-1.77 (m, 3H).

### Example 96

### 3-[2-Chloro-5-methyl-3′-(methylsulfonyl)biphenyl-4-yl]-4-hydroxy-1-oxa-8-azaspiro[4.5]dec-3-en-2-one hydrochloride

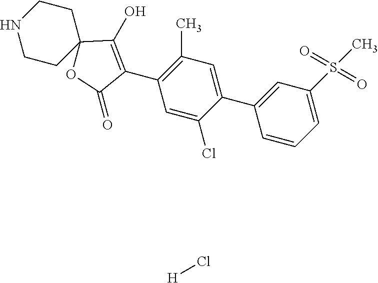

A solution of 1.15 g (1.97 mmol) of benzyl 3-[2-chloro-5-methyl-3′-(methylsulfonyl)biphenyl-4-yl]-4-hydroxy-2-oxo-1-oxa-8-azaspiro[4.5]dec-3-ene-8-carboxylate from example 72 in 10 ml of trifluoroacetic acid is stirred for 2 hours at 40° C. The reaction solution is concentrated on a rotary evaporator and the residue obtained is separated by preparative HPLC (eluent: acetonitrile/water+1 vol % 1N hydrochloric acid). 700 mg (1.4 mmol, 68% th.) of product are obtained. LC-MS (Method 1): Rₜ=1.49 min. MS (ESIpos): m/z=448 (M+H)⁺. ¹H NMR (300 MHz, DMSO-d₆): δ=8.9-8.67 (m, 1H), 8.67-8.47 (m, 1H), 7.99-7.9 (m, 2H), 7.86-7.7 (m, 2H), 7.4 (s, 1H), 7.29 (s, 1H), 3.28 (s, 3H), 3.18-3.0 (m, 3H), 2.3-2.07 (m, 6H), 1.72 (d, 2H).

GWP7:

The piperidine derivative (1 eq.), potassium carbonate (3 eq.) and the bromo derivative (1.1 eq.) are stirred in DMF for 12 hours at 50° C. After cooling the reaction solution is separated preparative HPLC.

GWP8:

The corresponding acid (1.6 eq.), HATU (1.5 eq.) and N,N-diisopropylethylamine are provided in DMF and the amine (1 eq.) is added. This solution is stirred for 2 hours at room temperature. The reaction mixture is quenched with 1 molar hydrochloric acid and separated by preparative HPLC.

GWP9:

The piperidine derivative (1 eq.) is dissolved in pyridine, the corresponding acid chloride (1.5 eq.) is added and the mixture is stirred for 2 hours at 80° C. The reaction solution is separated by preparative HPLC.

GWP10:

The piperidine derivative (1 eq.) is dissolved in DMF and N,N-diisopropylethylamine (3 eq.) is added. The corresponding acid chloride (1.3 eq.) is added dropwise and the mixture is stirred for 1 hour at RT. The reaction solution is separated by preparative HPLC.

The following compounds are prepared from example 94 to 96, the respective GWP and the general preparative information.

| | | | Analysis |
|---|---|---|---|
| | | Prepared from | LC-MS (Method) |
| Ex. | | by GWP | Rₜ [min] |
| No. | Structure | Yield [th.] | MS: m/z |
| 97 | 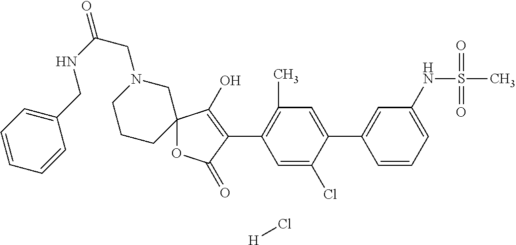 | Example 94 GWP7 60% | LC-MS (1) 1.83 ESI+: 610 (M + H)⁺ |
| 98 | 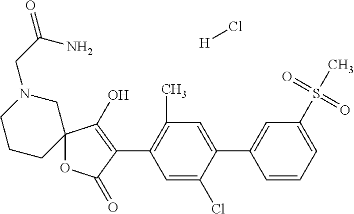 | Example 95 GWP7 14% | LC-MS (3) 1.58 ESI+: 505 (M + H)⁺ |
| 99 | 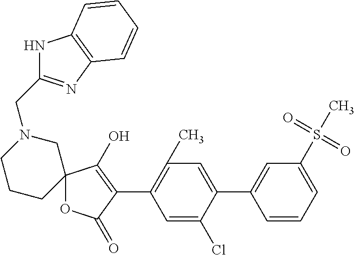 | Example 95 GWP7 62% | LC-MS (2) 1.57 ESI+: 578 (M + H)⁺ |
| 100 | 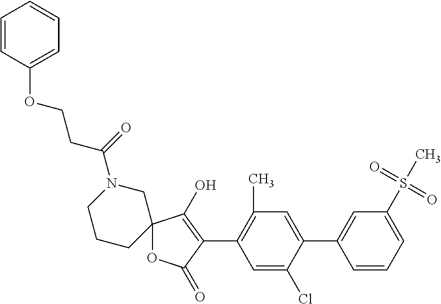 | Example 95 GWP8 80% | LC-MS (2) 2.12 ESI+: 596 (M + H)⁺ |
| 101 | 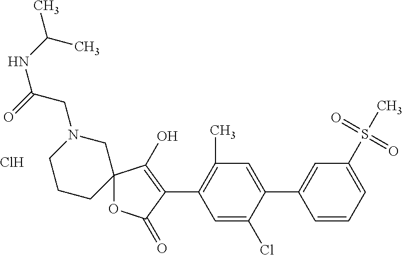 | Example 95 GWP7 88% | LC-MS (2) 1.50 ESI+: 547 (M + H)⁺ |
| 102 | 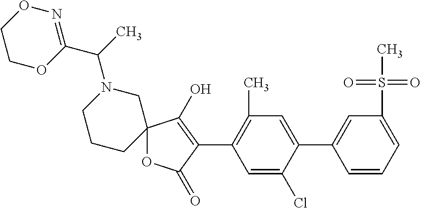 | Example 95 GWP7 23% | LC-MS (1) 1.63 ESI+: 561 (M + H)⁺ |
| 103 | 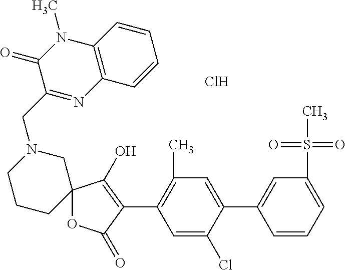 | Example 95 GWP8 23% | LC-MS (1) 1.76 ESI+: 620 (M + H)⁺ |
| 104 | 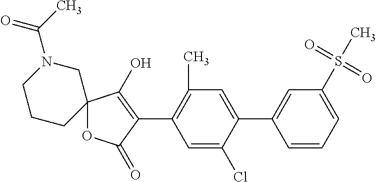 | Example 95 GWP8 55% | LC-MS (2) 1.57 ESI+: 490 (M + H)⁺ |
| 105 | 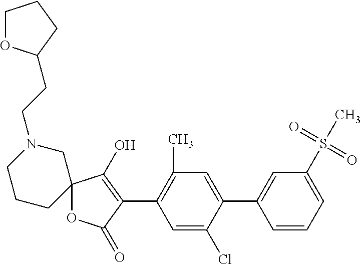 | Example 95 GWP8 95% | LC-MS (2) 1.77 ESI+: 574 (M + H)⁺ |
| 106 | 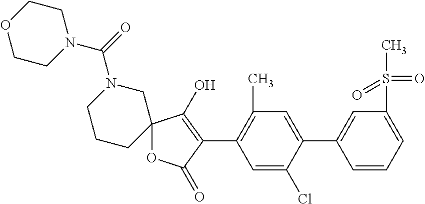 | Example 95 GWP10 33% | LC-MS (2) 1.68 ESI+: 561 (M + H)⁺ |
| 107 | 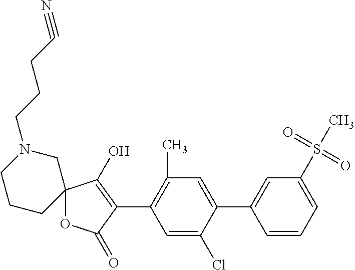 | Example 95 GWP8 98% | LC-MS (1) 1.84 ESI+: 543 (M + H)⁺ |
| 108 | 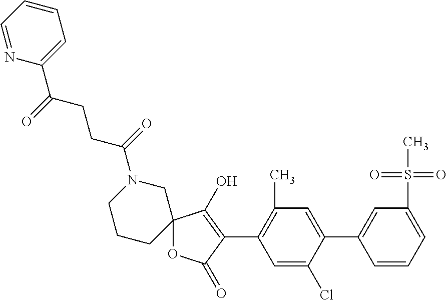 | Example 95 GWP8 80% | LC-MS (1) 1.94 ESI+: 609 (M + H)⁺ |
| 109 | 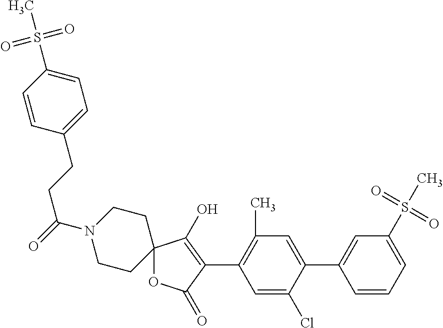 | Example 95 GWP8 89% | LC-MS (1) 1.91 ESI+: 658 (M + H)⁺ |
| 110 | 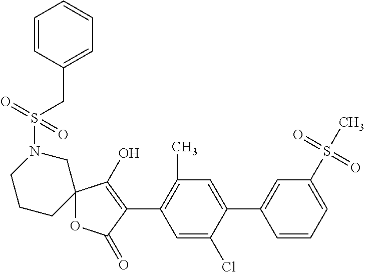 | Example 95 GWP9 60% | LC-MS (3) 2.55 ESI+: 602 (M + H)⁺ |
| 111 | 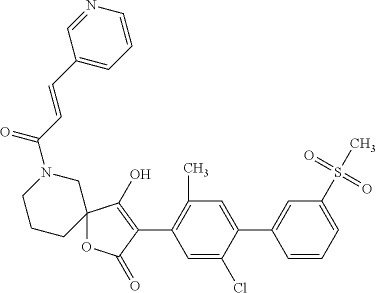 | Example 95 GWP8 22% | LC-MS (3) 1.78 ESI+: 579 (M + H)⁺ |
| 112 | 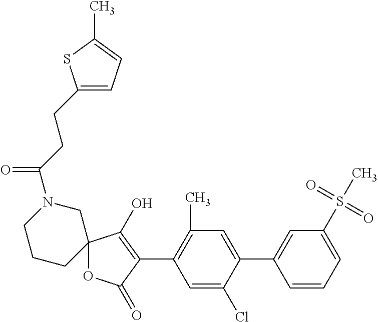 | Example 95 GWP8 70% | LC-MS (2) 2.33 ESI+: 619 (M + H)⁺ |
| 113 | 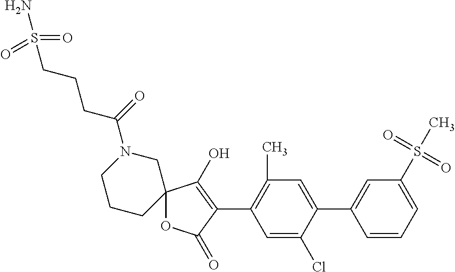 | Example 95 GWP8 95% | LC-MS (2) 1.53 ESI+: 597 (M + H)⁺ |
| 114 | 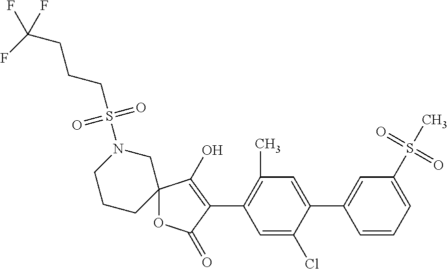 | Example 95 GWP9 21% | LC-MS (2) 2.24 ESI+: 622 (M + H)⁺ |
| 115 | 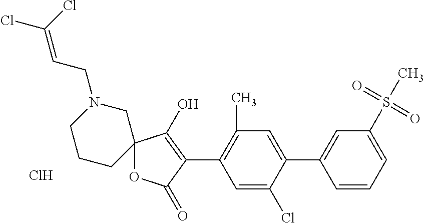 | Example 95 GWP7 80% | LC-MS (3) 1.85 ESI+: 555 (M + H)⁺ |
| 116 | 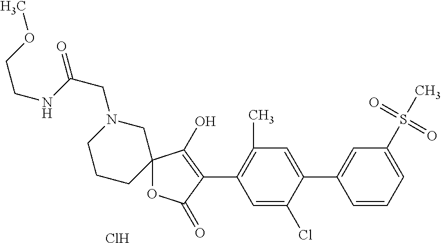 | Example 95 GWP7 95% | LC-MS (3) 1.64 ESI+: 563 (M + H)⁺ |
| 117 | 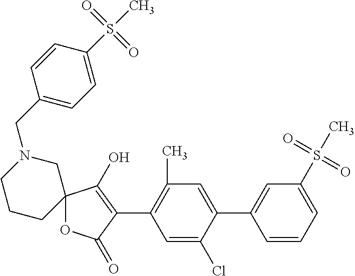 | Example 95 GWP7 62% | LC-MS (1) 1.68 ESI+: 616 (M + H)⁺ |
| 118 | 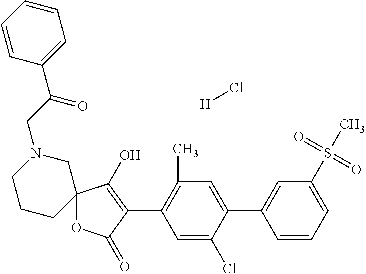 | Example 95 GWP7 37% | LC-MS (2) 1.64 ESI+: 566 (M + H)⁺ |
| 119 | 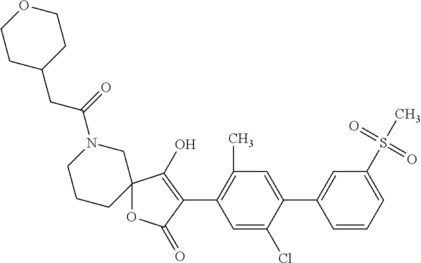 | Example 95 GWP8 32% | LC-MS (3) 2.03 ESI+: 574 (M + H)⁺ |
| 120 | 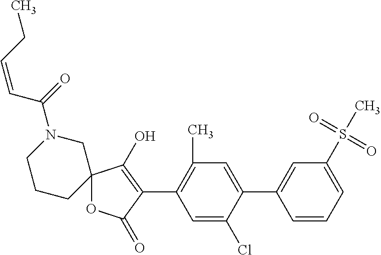 | Example 95 GWP8 29% | LC-MS (3) 2.24 ESI+: 530 (M + H)⁺ |
| 121 | 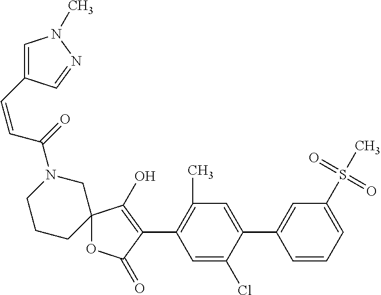 | Example 95 GWP8 14% | LC-MS (1) 1.87 ESI+: 582 (M + H)⁺ |
| 122 | 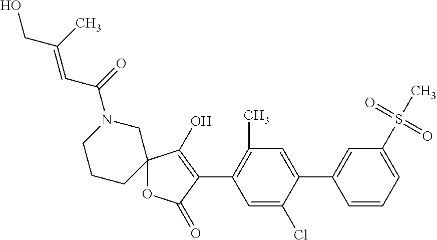 | Example 95 GWP8 16% | LC-MS (1) 1.75 ESI+: 546 (M + H)⁺ |
| 123 | 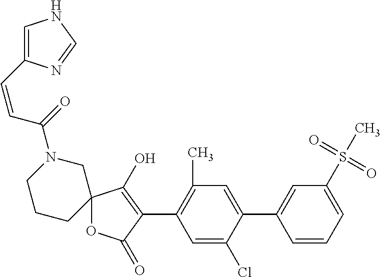 | Example 95 GWP8 75% | LC-MS (1) 1.52 ESI+: 568 (M + H)⁺ |
| 124 | 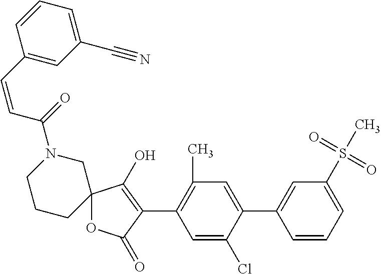 | Example 95 GWP8 37% | LC-MS (3) 2.35 ESI+: 603 (M + H)⁺ |
| 125 | 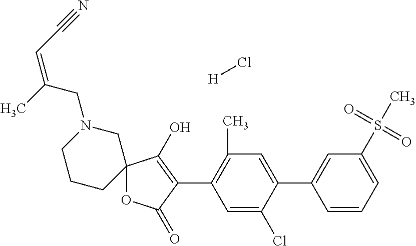 | Example 95 GWP7 74% | LC-MS (3) 1.76 ESI+: 527 (M + H)⁺ |
| 126 | 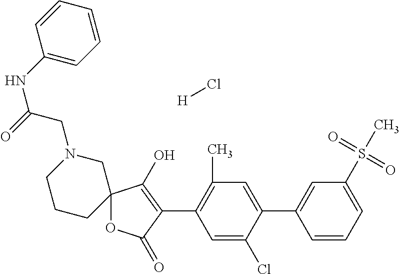 | Example 95 GWP7 85% | LC-MS (3) 1.93 ESI+: 581 (M + H)⁺ |
| 127 | 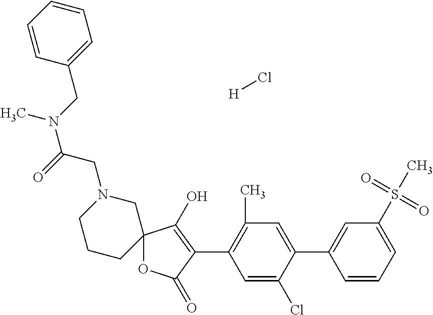 | Example 95 GWP7 74% | LC-MS (3) 1.96 ESI+: 609 (M + H)⁺ |
| 128 | 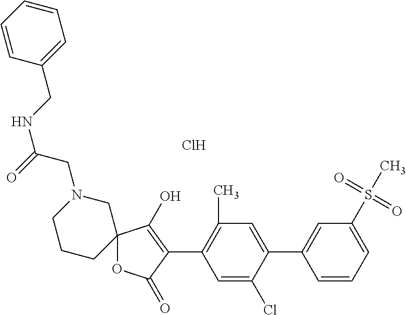 | Example 95 GWP7 78% | LC-MS (3) 1.92 ESI+: 595 (M + H)⁺ |
| 129 | 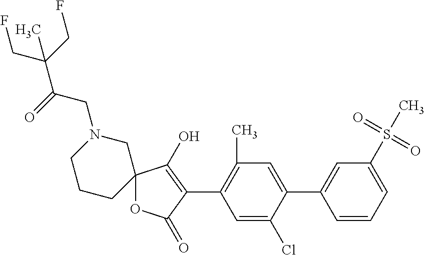 | Example 95 GWP7 22% | LC-MS (1) 1.77 ESI+: 582 (M + H)⁺ |
| 130 | 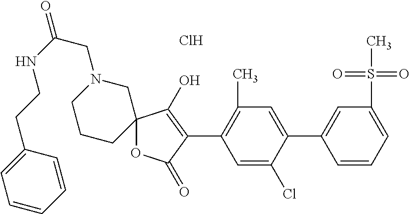 | Example 95 GWP7 83% | LC-MS (1) 1.86 ESI+: 609 (M + H)⁺ |
| 131 | 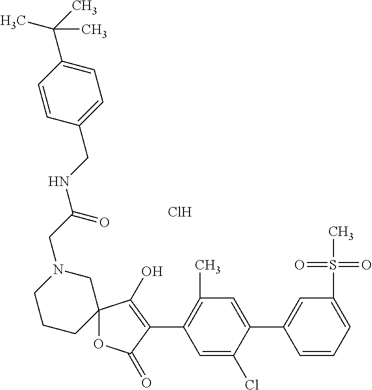 | Example 95 GWP7 66% | LC-MS (1) 2.18 ESI+: 651 (M + H)⁺ |
| 132 | 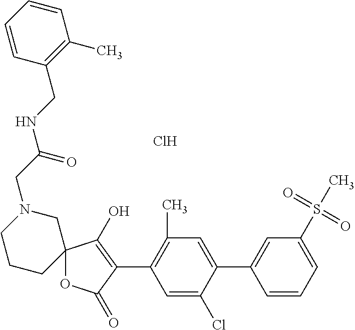 | Example 95 GWP7 72% | LC-MS (1) 1.90 ESI+: 609 (M + H)⁺ |
| 133 | 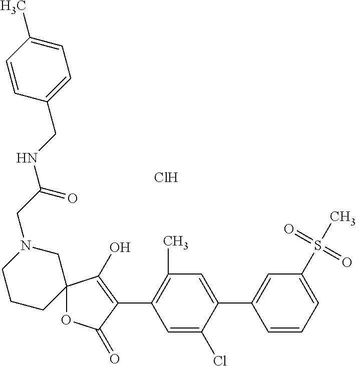 | Example 95 GWP7 74% | LC-MS (1) 1.92 ESI+: 609 (M + H)⁺ |
| 134 | 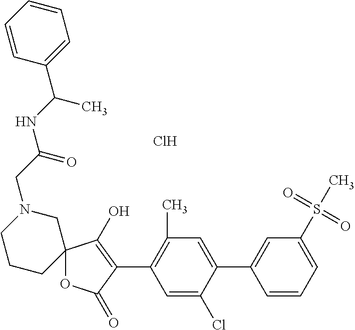 | Example 95 GWP7 87% | LC-MS (1) 1.88 ESI+: 609 (M + H)⁺ |
| 135 | 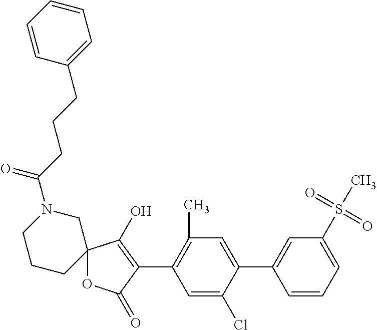 | Example 95 GWP8 64% | LC-MS (1) 2.47 ESI+: 594 (M + H)⁺ |
| 136 | 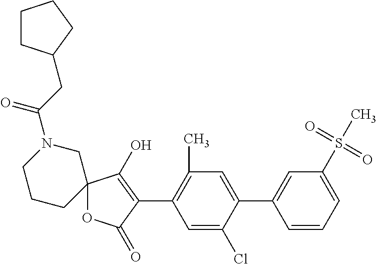 | Example 95 GWP8 52% | LC-MS (1) 2.42 ESI+: 558 (M + H)⁺ |
| 137 | 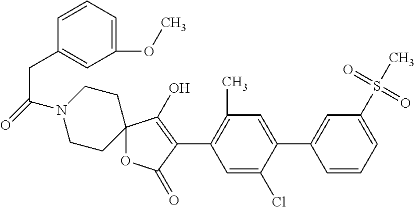 | Example 96 GWP9 7% | LC-MS (3) 2.33 ESI+: 596 (M + H)⁺ |
| 138 | 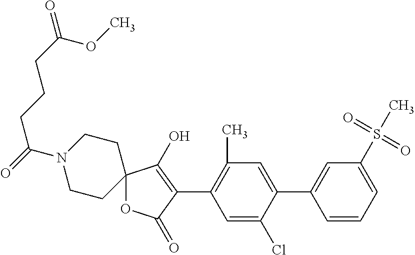 | Example 96 GWP9 45% | LC-MS (1) 1.96 ESI+: 576 (M + H)⁺ |
| 139 | 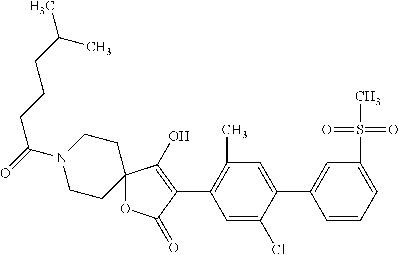 | Example 96 GWP8 61% | LC-MS (3) 2.52 ESI+: 560 (M + H)⁺ |
| 140 | 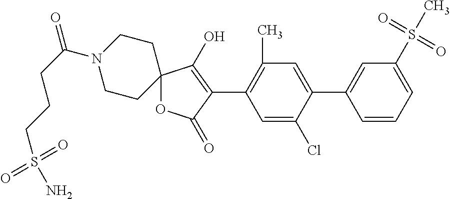 | Example 96 GWP8 26% | LC-MS (2) 1.58 ESI+: 597 (M + H)⁺ |
| 141 | 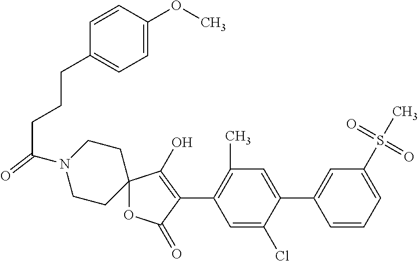 | Example 96 GWP8 78% | LC-MS (3) 2.47 ESI+: 624 (M + H)⁺ |
| 142 | 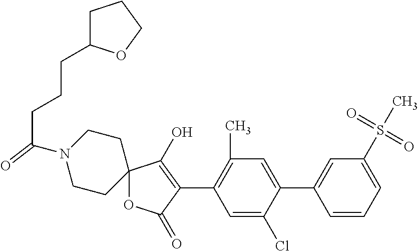 | Example 96 GWP8 69% | LC-MS (1) 1.97 ESI+: 574 (M + H)⁺ |
| 143 | 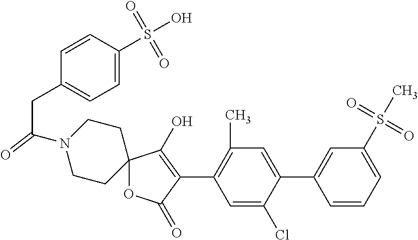 | Example 96 GWP9 28% | LC-MS (3) 2.25 ESI+: 646 (M + H)⁺ |
| 144 | 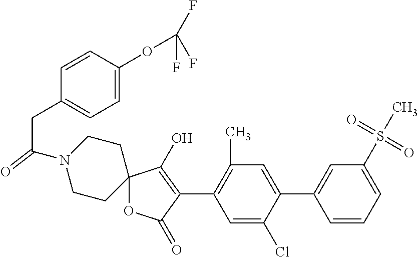 | Example 96 GWP9 18% | LC-MS (1) 2.33 ESI+: 650 (M + H)⁺ |
| 145 | 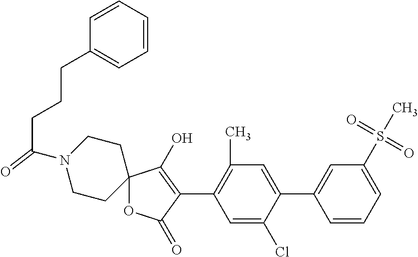 | Example 96 GWP8 79% | LC-MS (3) 2.48 ESI+: 594 (M + H)⁺ |
| 146 | 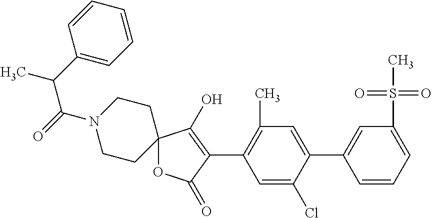 | Example 96 GWP9 66% | LC-MS (3) 2.43 ESI+: 580 (M + H)⁺ |
| 147 | 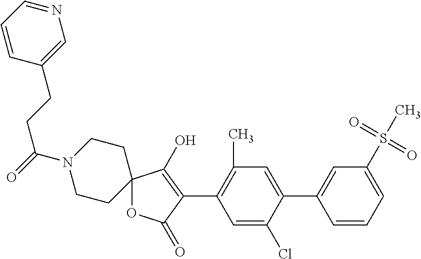 | Example 96 GWP8 50% | LC-MS (3) 1.70 ESI+: 581 (M + H)⁺ |
| 148 | 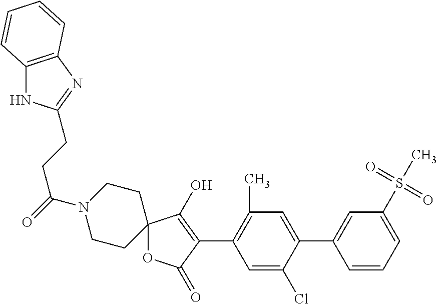 | Example 96 GWP8 18% | LC-MS (2) 1.48 ESI+: 620 (M + H)⁺ |
| 149 | 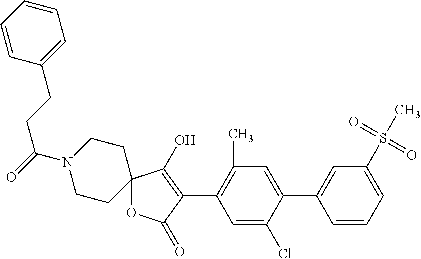 | Example 96 GWP8 90% | LC-MS (2) 2.16 ESI+: 580 (M + H)⁺ |
| 150 | 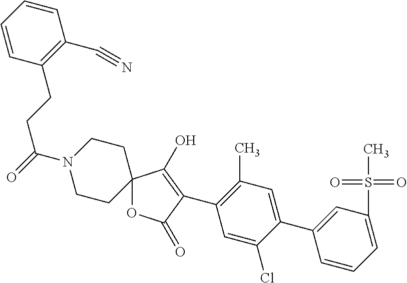 | Example 96 GWP8 40% | LC-MS (2) 2.07 ESI+: 605 (M + H)⁺ |
| 151 | 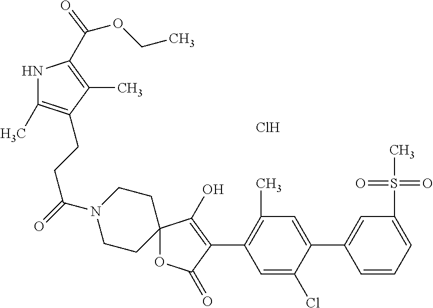 | Example 96 GWP8 92% | LC-MS (2) 2.15 ESI+: 669 (M + H)⁺ |
| 152 | 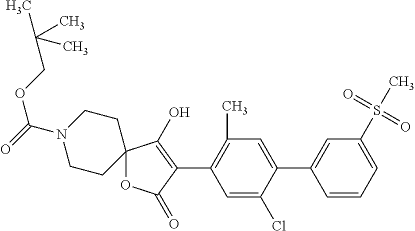 | Example 96 GWP10 89% | LC-MS (2) 2.34 ESI+: 562 (M + H)⁺ |
| 153 | 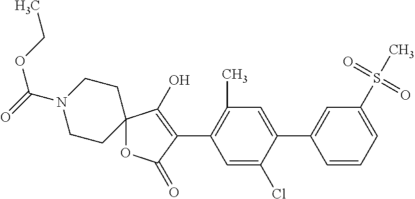 | Example 96 GWP9 44% | LC-MS (2) 1.98 ESI+: 520 (M + H)⁺ |
| 154 | 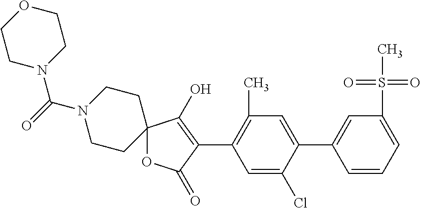 | Example 96 GWP10 84% | LC-MS (2) 1.75 ESI+: 561 (M + H)⁺ |
| 155 | 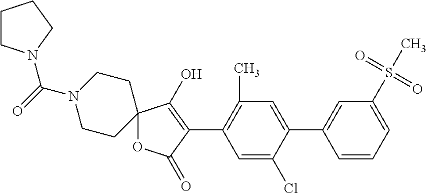 | Example 96 GWP10 74% | LC-MS (3) 2.27 ESI+: 545 (M + H)⁺ |
| 156 | 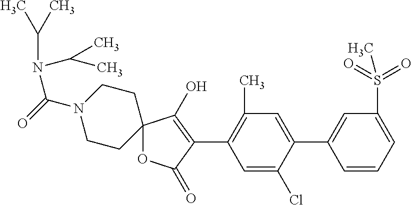 | Example 96 GWP10 60% | LC-MS (1) 2.27 ESI+: 575 (M + H)⁺ |
| 157 | 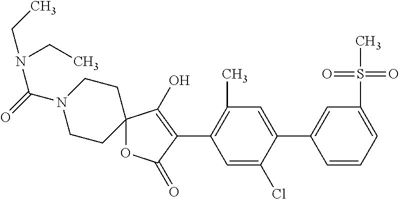 | Example 96 GWP10 90% | LC-MS (3) 2.39 ESI+: 547 (M + H)⁺ |
| 158 | 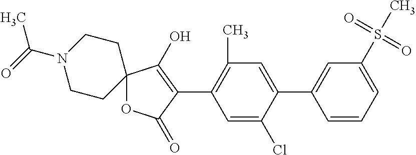 | Example 96 GWP8 14% | LC-MS (1) 1.81 ESI+: 490 (M + H)⁺ |
| 159 | 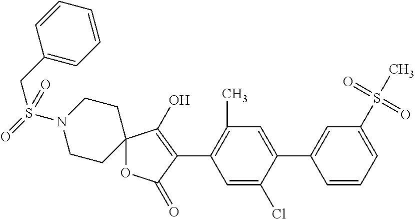 | Example 96 GWP9 48% | LC-MS (2) 2.28 ESI+: 602 (M + H)⁺ |
| 160 | 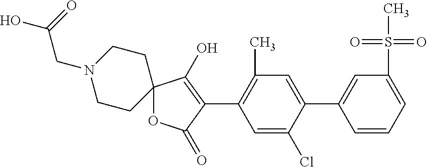 | Example 96 GWP7 68% | LC-MS (2) 1.25 ESI+: 506 (M + H)⁺ |

### Example 161

### 2-{3-[2-Chloro-5-methyl-3′-(methylsulfonyl)biphenyl-4-yl]-4-hydroxy-2-oxo-1-oxa-7-azaspiro[4.5]dec-3-en-7-yl}-N-(pyridin-4-ylmethyl)acetamide

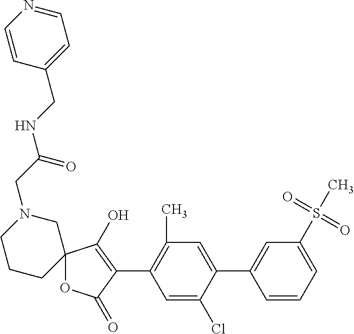

49.8 mg (0.313 mmol) of HATU, 0.061 mml (0.349 mmol) of N,N-diisopropylethylamine and 14.2 mg (0.313 mmol) of 4-(aminomethyl)pyridine are added to a solution of 47 mg (0.087 mmol) of {3-[2-chloro-5-methyl-3′-(methylsulfonyl)biphenyl-4-yl]-4-hydroxy-2-oxo-1-oxa-7-azaspiro[4.5]dec-3-en-7-yl}acetic acid from example 160 in 5 ml of DMF and the mixture is stirred overnight at room temperature. The reaction solution is quenched with 1 ml of 1 molar hydrochloric acid and separated by preparative HPLC. 15 mg (0.025 mmol, 28% th.) of product are obtained. LC-MS (Method 1): Rₜ=1.32 min. MS (ESIpos): m/z=596 (M+H)⁺

GWP11:

HATU (1.5 eq), N,N-diisopropylethylamine (4 eq.) and the corresponding amine (1.5 eq.) are added to a solution of the acid (1 eq.) in DMF and the mixture is stirred overnight at room temperature. The reaction solution is quenched with 1 molar hydrochloric acid and separated by preparative HPLC.

The following compounds are prepared in analogy to example 161 and GWP 11:

| | | | Analysis |
|---|---|---|---|
| | | | LC-MS (Method) |
| Ex. | | Prepared from | Rₜ [min] |
| No. | Structure | Yield [th.] | MS: m/z |
| 162 | 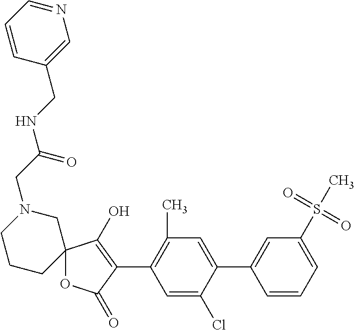 | Example 160 38% | LC-MS (3) 1.43 ESI+: 596 (M + H)⁺ |
| 163 | 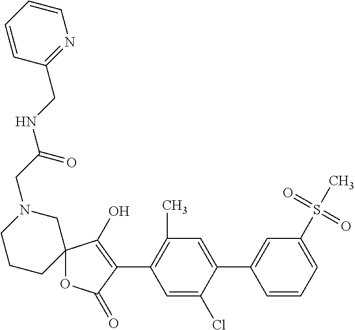 | Example 160 42% | LC-MS (3) 1.56 ESI+: 596 (M + H)⁺ |
| 164 | 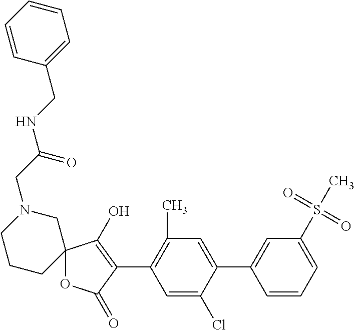 | Example 128 1. Stereoisomer 22% | HPLC chiral (1) 9.86 |
| 165 | 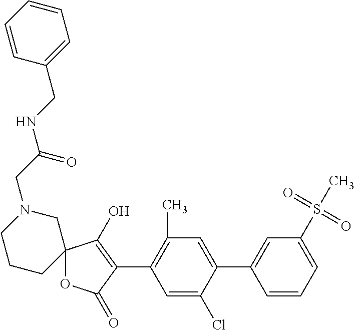 | Example 128 2. Stereoisomer 23% | HPLC chiral (1) 14.05 |
| 166 | 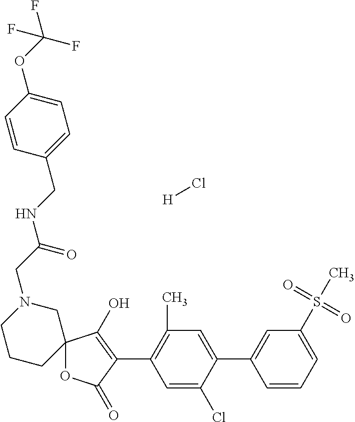 | Example 160 24% | LC-MS (1) 2.08 ESI+: 679 (M + H)⁺ |
| 167 | 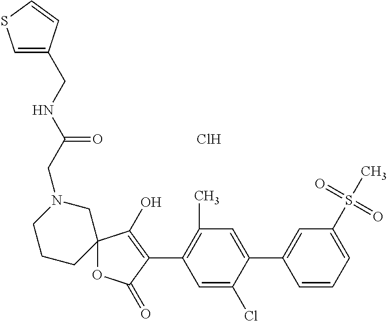 | Example 160 31% | LC-MS (3) 1.88 ESI+: 601 (M + H)⁺ |
| 168 | 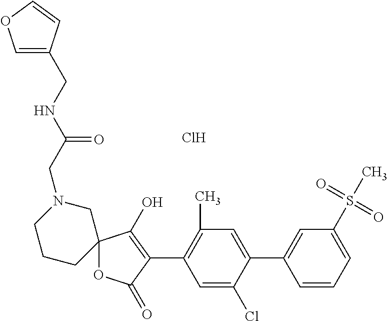 | Example 160 59% | LC-MS (3) 1.81 ESI+: 585 (M + H)⁺ |
| 169 | 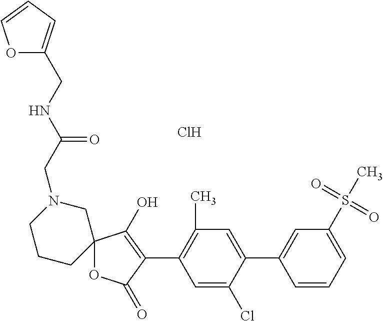 | Example 160 67% | LC-MS (3) 1.81 ESI+: 585 (M + H)⁺ |
| 170 | 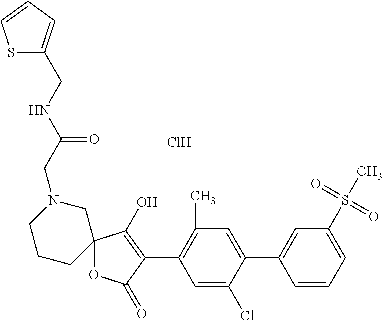 | Example 160 32% | LC-MS (3) 1.86 ESI+: 601 (M + H)⁺ |

### Example 171

### 3-(4′-Amino-2-chloro-5-methylbiphenyl-4-yl)-4-hydroxy-1-oxaspiro[4.5]dec-3-en-2-one

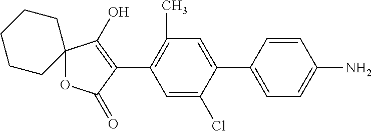

A solution of 145 mg (0.35 mmol) of 3-(2-chloro-5-methyl-4′-nitrobiphenyl-4-yl)-4-hydroxy-1-oxaspiro[4.5]dec-3-en-2-one from example 47 is provided in 15 ml of acetic acid and 136.9 mg (2.45 mmol) of iron powder are added. The reaction solution is stirred for 12 hours at 50° C. The suspension is filtered, washed with DMSO and the filtrate is concentrated on a rotary evaporator. The residue obtained is separated by preparative HPLC. 130 mg (0.34 mmol, 97% th.) of product are obtained. LC-MS (Method 2): Rₜ=2.05 min. MS (ESIpos): m/z=384 (M+H)⁺. ¹H NMR (300 MHz, DMSO-d₆): δ=7.52 8d, 2H), 7.38 (d, 2H), 7.31 (s, 2H), 2.18 (s, 3H), 2.05-1.87 (m, 2H), 1.8-1.43 (m, 7H), 1.37-1.16 (m, 1H).

GWP12:

The nitro compound (1 eq.) is provided in acetic acid and iron powder (7 eq.) is added. The reaction solution is stirred for 12 hours at 50° C. The suspension is filtered and the filtrate is concentrated on a rotary evaporator. The residue obtained is separated by preparative HPLC.

The following compounds are prepared in analogy to example 171 and GWP12:

| | | | Analysis |
|---|---|---|---|
| | | | LC-MS (Method) |
| Ex. | | Prepared from | Rₜ [min] |
| No. | Structure | Yield [th.] | MS: m/z |
| 172 | 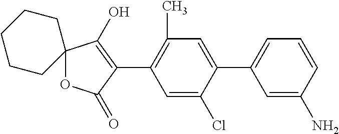 | Example 40 57% | LC-MS (3) 2.36 ESI+: 384 (M + H)⁺ |
| 173 | 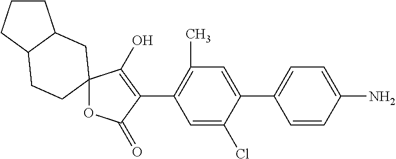 | Example 89 71% | LC-MS (3) 2.68 ESI+: 424 (M + H)⁺ |
| 174 | 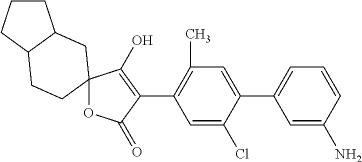 | Example 88 77% | LC-MS (3) 2.67 ESI+: 424 (M + H)⁺ |
| 175 | 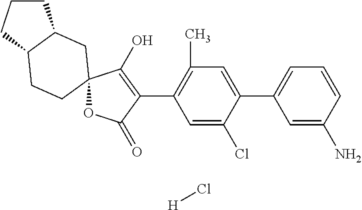 | Example 90 41% | LC-MS (3) 2.61 ESI+: 424 (M + H)⁺ |

### Example 176

### N-[2′-Chloro-4′-(4-hydroxy-2-oxo-1-oxaspiro[4.5]dec-3-en-3-yl)-5′-methylbiphenyl-3-yl]methanesulfonamide

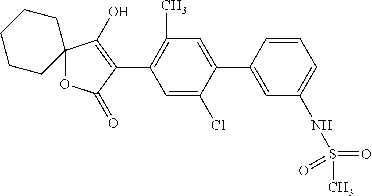

0.1 ml (1.27 mmol) of methanesulfonyl chloride is added to a solution of 445 mg (1.16 mmol) of 3-(3′-amino-2-chloro-5-methylbiphenyl-4-yl)-4-hydroxy-1-oxaspiro[4.5]dec-3-en-2-one from example 172 in 15 ml of pyridine and the mixture is stirred for 12 hours at 40° C. The solvent is evaporated on a rotary evaporator and the residue obtained separated by preparative HPLC. 386 mg (0.84 mmol, 72% th.) of product are obtained. LC-MS (Method 2): Rₜ=2.19 min. MS (ESIpos): m/z=462 (M+H)⁺. ¹H NMR (300 MHz, DMSO-d₆): δ=12.4 (s, 1H), 9.91 (s, 1H), 7.49-7.38 (m, 1H), 7.34-7.21 (m, 4H), 7.16 (d, 1H), 3.04 (s, 3H), 2.18 (s, 3H), 1.99-1.83 (m, 2H), 1.81-1.48 (m, 6H), 1.35-1.17 (m, 1H).

GWP13:

The amine (1 eq.) is dissolved in pyridine and the corresponding acid chloride is added. The reaction solution is stirred for 12 hours at 40° C. and after cooling separated by preparative HPLC.

GWP14:

The corresponding acid (1.6 eq.), HATU (1.5 eq.) and DMAP (4 eq.) are provided in DMF and the amine (1 eq.) is added. The mixture is stirred for 3 hours at room temperature and subsequently purified by preparative HPLC.

GWP15:

The amine (0.164 mmol, 1 eq.) is dissolved in pyridine and the corresponding sulfonyl chloride (3 eq.) is added and the mixture is stirred for 18 hours at 40° C. After cooling, the mixture is extracted and separated by preparative HPLC.

The following compounds are prepared in analogy to example 176 and the respective GWP13 and GWP14:

| | | | Analysis |
|---|---|---|---|
| | | | LC-MS (Method) |
| Ex. | | Prepared from | Rₜ [min] |
| No. | Structure | Yield [th.] | MS: m/z |
| 177 | 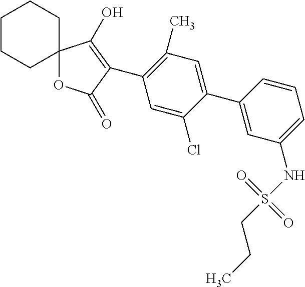 | Example 172 75% | LC-MS (3) 2.63 ESI+: 490 (M + H)⁺ |
| 178 | 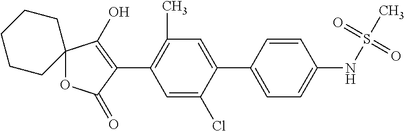 | Example 172 38% | LC-MS (3) 2.45 ESI+: 462 (M + H)⁺ |
| 179 | 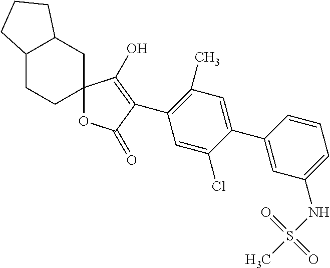 | Example 174 5% | LC-MS (3) 2.79 ESI+: 502 (M + H)⁺ |
| 180 | 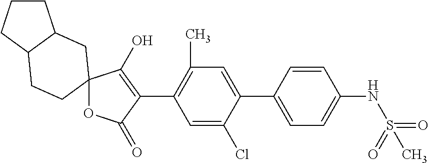 | Example 173 32% | LC-MS (3) 2.76 ESI+: 502 (M + H)⁺ |
| 181 | 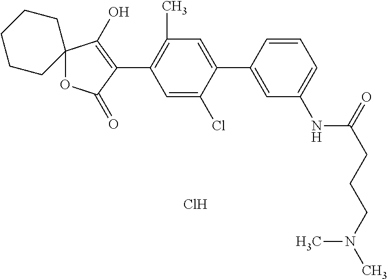 | Example 172 28% | LC-MS (3) 1.79 ESI+: 497 (M + H)⁺ |
| 182 | 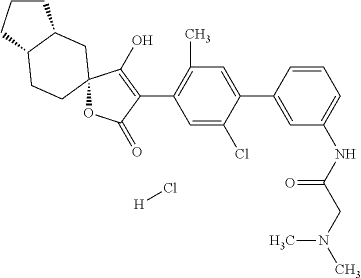 | Example 175 59% | LC-MS (4) 3.29 ESI+: 509 (M + H)⁺ |
| 183 | 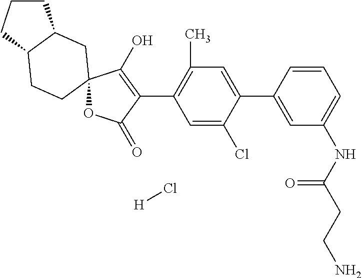 | Example 175 34% | LC-MS (1) 1.87 ESI+: 495 (M + H)⁺ |

### Example 184

### N-[2′-Chloro-4′-(4-hydroxy-2-oxo-1-oxaspiro[4.5]dec-3-en-3-yl)-4,5′-dimethylbiphenyl-3-yl]acetamide

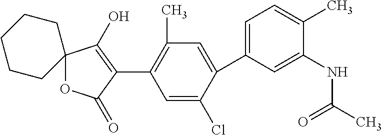

155 mg (0.308 mmol) of 3-(2-chloro-4′,5-dimethyl-3′-nitrobiphenyl-4-yl)-4-hydroxy-1-oxaspiro[4.5]-dec-3-en-2-one from Example 83 is provided in acetic acid and 120.4 mg (2.2 mmol) of iron powder are added. The mixture is stirred for 3 hours at 50° C. The reaction solution is concentrated on a rotary evaporator and the resulting intermediate is taken up in 4 ml of pyridine. 42 mg (0.369 mmol) of methanesulfonyl chloride is added and the mixture is stirred for 3 hours at 50° C. 69 mg (0.16 mmol, 48% th.) of product are obtained. LC-MS (Method 2): Rₜ=2.18 min. MS (ESIpos): m/z=440 (M+H)⁺. ¹H NMR (400 MHz, DMSO-d₆): δ=9.35 (s, 1H), 7.54 (s, 1H), 7.34-7.23 (m, 3H), 7.15 (d, 1H), 2.25 (s, 3H), 2.17 (s, 3H), 2.08 (s, 3H), 1.98-1.84 (m, 2H), 1.8-1.5 (m, 7H), 1.34-1.19 (m, 1H).

### Example 185

### N-[2′-Chloro-4′-(4-hydroxy-2-oxo-1-oxaspiro[4.5]dec-3-en-3-yl)-5′-methylbiphenyl-3-yl]-N-methylmethanesulfonamide

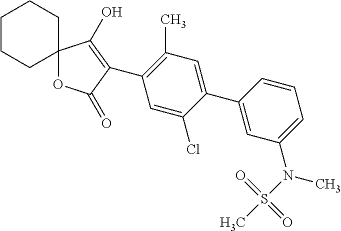

5.7 mg (0.143 mmol) of sodium hydride and 9.2 mg (0.065 mmol) of iodomethane are added to a solution of 30 mg (0.065 mmol) of N-[2′-chloro-4′-(4-hydroxy-2-oxo-1-oxaspiro[4.5]dec-3-en-3-yl)-5′-methylbiphenyl-3-yl]methanesulfonamide from example 176 in 2 ml DMF with the exclusion of oxygen and the reaction mixture is stirred for 4 hours at room temperature. The reaction mixture is separated by preparative HPLC. 14 mg (0.03 mmol, 45% th.) of product are obtained. LC-MS (Method 3): Rₜ=2.62 min. MS (ESIpos): m/z=476 (M+H)⁺. ¹H NMR (400 MHz, DMSO-d₆): δ=12.39 (s, 1H), 7.56-7.29 (m, 5H), 3.4-3.22 (m, 3H, partly masked by water), 2.97 (s, 3H), 2.19 (s, 3H), 1.96-1.84 (m, 2H), 1.8-1.66 (m, 3H), 1.66-1.5 (m, 4H), 1.33-1.18 (m, 1H).

### Example 186

### 5-{3-[2-Chloro-5-methyl-3′-(methylsulfonyl)biphenyl-4-yl]-4-hydroxy-2-oxo-1-oxa-8-azaspiro[4.5]-dec-3-en-8-yl}-5-oxopentanoic acid

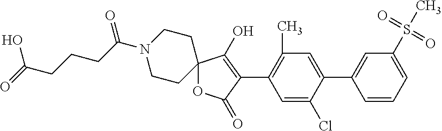

0.4 ml of a 50% sodium hydroxide solution are added to a solution of 60 mg (0.104 mmol) of methyl 5-{3-[2-chloro-5-methyl-3′-(methylsulfonyl)biphenyl-4-yl]-4-hydroxy-2-oxo-1-oxa-8-azaspiro[4.5]dec-3-en-8-yl}-5-oxopentanoate from example 138 in 2 ml of ethanol and 5 ml of THF and the mixture is stirred for 1 hour at room temperature. The mixture is acidified with 1 molar hydrochloric acid and concentrated on a rotary evaporator. The residue obtained is separated by preparative HPLC. 27 mg (0.05 mmol, 46% th.) of product are obtained. LC-MS (Method 2): Rₜ=1.60 min. MS (ESIpos): m/z=562 (M+H)⁺. ¹H NMR (400 MHz, DMSO-d₆): δ=12.06 (s, 1H), 8.03-7.94 (m, 2H), 7.89-7.74 (m, 2H), 7.42 (d, 2H), 4.51 (d, 1H), 3.98 (d, 1H), 3.3-3.27 (s, 3H, partly masked by water), 2.84 (t, 1H), 2.41 (t, 2H), 2.29 (t, 2H), 2.23 (s, 3H), 2.17-2.05 (m, 2H), 2.04-1.93 (m, 1H), 1.8-1.62 (m, 4H).

The following compounds are prepared in analogy to example 17, the respective GWP and the general preparative information:

| | | | Analysis |
|---|---|---|---|
| | | prepared from | LC-MS (Method) |
| Ex. | | by GWP | Rₜ [min] |
| No. | Structure | Yield [d. Th.] | MS: m/z |
| 187 | 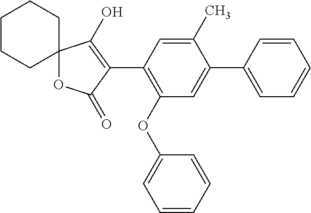 | Example 53A GWP5 33% | LC-MS (2) 2.81 ESI+: 446 (M + H)⁺ |
| 188 | 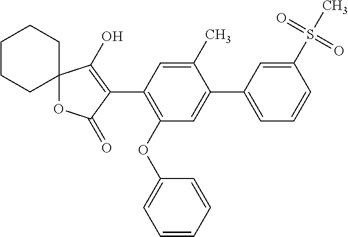 | Example 53A GWP5 7% | LC-MS (2) 2.45 ESI+: 524 (M + H)⁺ |
| 189 | 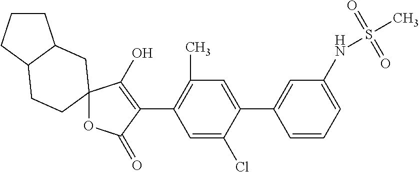 | Example 49A GWP5 49% | LC-MS (1) 2.73 ESI−: 500 (M − H)⁻ |

The following compounds are prepared in analogy to example 176 and GWP15:

| | | | Analysis |
|---|---|---|---|
| | | | LC-MS (Method) |
| Ex. | | Prepared from | Rₜ [min] |
| No. | Structure | Yield [th.] | MS: m/z |
| 190 | 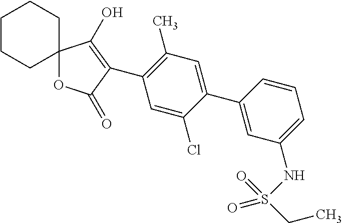 | Example 172 33% | LC-MS (1) 2.47 ESI−: 474 (M − H)⁻ |
| 191 | 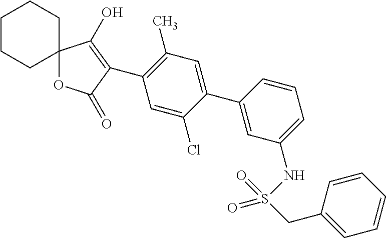 | Example 172 56% | LC-MS (1) 2.70 ESI−: 536 (M − H)⁻ |
| 192 | 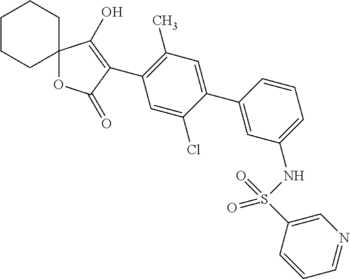 | Example 172 37% | LC-MS (1) 2.43 ESI+: 525 (M + H)⁺ |

### Example 193

### N-[2′-Chloro-4′-(4-hydroxy-2-oxo-1-oxaspiro[4.5]dec-3-en-3-yl)-5′-methylbiphenyl-3-yl]ethenesulfonamide

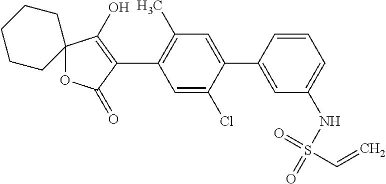

300 mg (0.657 mmol) of the compound from Example 172 are stirred for 4 h at 40° C. in 10 ml of THF with 60 mg (0.657 mmol) of DMAP, 0.45 ml (2.63 mmol) of DIPEA and 0.148 ml (1.41 mmol) of 2-chloroethylsulfonyl chloride. After this a further 0.45 ml (2.63 mmol) of DIPEA and 0.148 ml (1.41 mmol) of 2-chloroethylsulfonyl chloride are added and the mixture is stirred for 6 h at 60° C. 6.5 ml of 1 N hydrochloric acid and a saturated sodium chloride solution are added to the mixture and the mixture is extracted three times with ethyl acetate. The combined organic phases are dried over sodium sulfate, evaporated and the residue is purified by preparative HPLC. 101 mg (30% th.) of a solid are obtained. LC-MS (Method 2): Rₜ=2.28 min. MS (ESIpos): m/z=474 (M+H)⁺. ¹H NMR (300 MHz, DMSO-d₆): δ=12.4 (b, 1H), 10.18 (s, 1H), 7.45-7.38 (m, 1H), 7.33-7.10 (m, 5H), 6.87-6.78 (d, 1H), 6.18-6.06 (dd, 1H), 2.18 (s, 3H), 1.99-1.85 (m, 2H), 1.81-1.50 (m, 7H), 1.35-1.17 (m, 1H).

### Example 194

### 2-Amino-N-[2′-chloro-4′-(4-hydroxy-2-oxo-1-oxaspiro[4.5]dec-3-en-3-yl)-5′-methylbiphenyl-3-yl]ethanesulfonamide

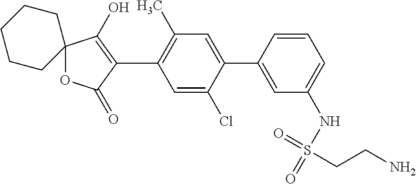

30 mg (0.056 mmol) of the compound from example 193 are left to stand for 3 weeks in 4 ml of 7 N methanolic ammonia at RT. The reaction mixture is evaporated to dryness and purified by preparative HPLC (gradient acetonitrile/water (10:90 to 90:10)) without the addition of acid. 12.5 mg (41% th.) of a solid are obtained. LC-MS (Method 3): Rₜ=1.75 min. MS (ESIpos): m/z=491 (M+H)⁺

### Example 195

### N-[2′-Chloro-4′-(4-hydroxy-2-oxo-1-oxaspiro[4.5]dec-3-en-3-yl)-5′-methylbiphenyl-3-yl]-2-(dimethylamino)ethanesulfonamide

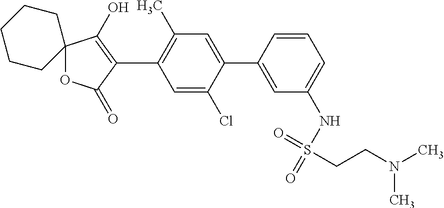

42.6 mg (0.079 mmol) of the compound from example 193 are dissolved in 0.5 ml of ethanol, 0.5 ml of a 40% aqueous solution of dimethylamine (3.95 mmol) are added and the mixture is stirred for 18 h at RT. The reaction mixture is concentrated to dryness and purified by preparative HPLC. 12 mg (29% th.) of the title compound are obtained. LC-MS (Method 3): Rₜ=1.85 min. MS (ESIpos): m/z=419 (M+H)⁺. ¹H-NMR (400 MHz, DMSO-d₆): δ=7.47-7.41 (m, 1H), 7.33 (s, 1H), 7.29 (s, 1H), 7.16-7.22 (m, 1H), 7.21-7.16 2 (m, 1H), 7.11 (m, 1H), 2.20 (s, 3H), 1.78-1.40 (m, 9H), 1.3-1.167.16-7.22 (m, 1H) (m, 1H).

The following compounds are prepared in analogy to the method for example 195:

| | | | Analysis |
|---|---|---|---|
| | | Starting material | LC-MS (Method) |
| Ex. | | Reagent | Rₜ [min] |
| No. | Structure | Yield [th.] | MS: m/z |
| 196 | 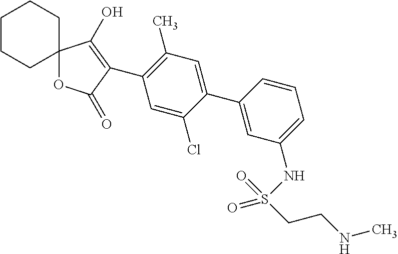 | Example 193 40% methylamine in water (54 eq.) 23% | LC-MS (1) 2.47 ESI−: 474 (M − H)⁻ |
| 197 | 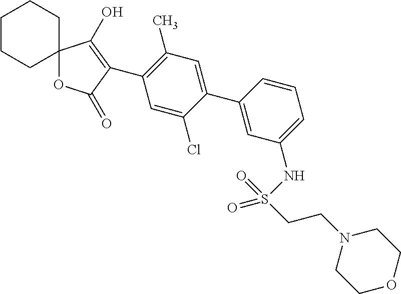 | Example 193 morpholine (4 eq.) 53% | LC-MS (1) 2.70 ESI−: 536 (M − H)⁻ |

The following compounds are prepared in analogy to the respective examples or GWP and the general preparative information:

| | | | Analysis |
|---|---|---|---|
| | | Prepared from | LC-MS (Method) |
| | | by GWP | Rₜ [min] |
| Ex. No. | Structure | Yield [th.] | MS: m/z |
| 198 | 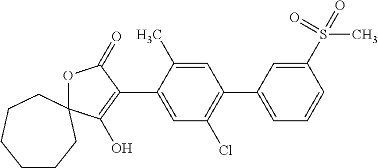 | Example 55A GWP5 76% | LC-MS (6) 2.79 ESI+: 461 (M + H)⁺ |
| 199 | 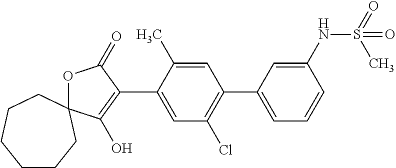 | Example 55A GWP5 83% | LC-MS (6) 2.76 ESI+: 476 (M + H)⁺ |
| 200 | 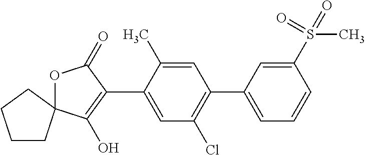 | Example 56A GWP4 23% | LC-MS (7) 3.21 ESI+: 433 (M + H)⁺ |
| 201 | 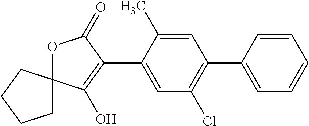 | Example 56A GWP4 34% | LC-MS (7) 3.65 ESI+: 355 (M + H)⁺ |
| 202 | 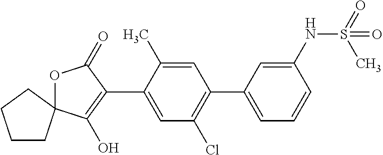 | Example 56A GWP5 28% | LC-MS (5) 3.40 ESI+: 448 (M + H)⁺ |
| 203 | 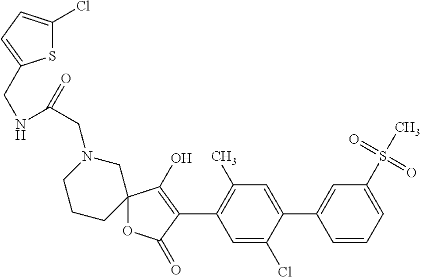 | Example 46A in Analogy to Ex. 94, GWP7, Ex. 161, GWP 5 39% | LC-MS (6) 2.09 ESI+: 635 (M + H)⁺ |

### B) EVALUATION OF THE PHYSIOLOGICAL ACTIVITY

The suitability of the compounds of the invention for the treatment of diseases caused by retroviruses can be shown by the following assay systems:

In Vitro Assays

Biochemical Protease Assay

For the determination of their in vitro activity on HIV proteases the test substances are dissolved in DMSO and serially diluted. In each case 0.5 μl of substance dilution, 20 μl of 0.2-1 nM HIV-1 protease wild type or mutant protein (e.g., multiresistant isolate “35513”: L10I, I15V, L19I, K20R, E35D, M36I, R41K, I54V, L63P, H69K, A71V, T74P, I84V, L89M, L90M, 193L, AscoProt Biotech, Prague, Czech Republic) in buffer 1 (50 mM sodium acetate pH 4.9, 0.02% BSA, 0.1 mM EDTA, 0.5 mM DTT) and 20 μl of 8 μM substrate (M1865 from Bachem, Bubendorf, Switzerland; Matayoshi et al., Science 1990, 247, 954-8) in buffer 1 are added successively to a 384 well microtiter plate (Greiner, Frickenhausen, Germany), incubated for 60-180 minutes at 32° C. and the fluorescence is measured (e.g., Tecan Safire, 340 nm extinction, 520 nm emission). IC₅₀ values are determined by graphical plotting the substance concentration against the percentage inhibition.

In this assay all exemplary embodiments have an IC₅₀ less than 10000 nM on HIV-1 protease wild type protein. The examples in Table 1 have an IC₅₀ value less than or equal to 100 nM.

TABLE 1
Ex. No.

9
10
11
12
13
14
15
17
18
19
21
22
23
24
26
27
30
31
32
33
34
35
36
37
38
40
41
42
43
44
47
48
49
50
51
52
53
54
55
56
57
58
59
60
61
62
69
70
71
72
75
77
79
82
84
85
86
87
88
89
91
92
93
97
99
100
101
102
104
105
106
107
108
109
110
111
112
113
114
116
120
121
122
123
124
126
128
129
130
132
133
134
135
136
138
139
140
141
142
143
144
145
147
152
153
155
156
157
158
161
162
163
164
165
166
167
168
169
170
171
175
176
177
178
180
181
182
183
184
185
188
189
190
191
192
193
198
199
200
201
202
203

Assembly Assay

The assembly assay records the late phase of HIV replication.

Day 1: 4×10e7 HEK293T cells of a logarithmically growing culture are seeded in 40 ml of medium (D-MEM with 4500 mg/l of glucose, 10% inactivated FKS, 2 mM glutamine, 100 μg/ml of penicillin/streptomycin) in a 225 cm² culture flask and incubated overnight in a cell culture incubator.

Day 2: The cells are co-transfected with each time 40 μg of pGJ3-RT K103N/Y181C and pcz-VSV-Gwt (provided by Jassoy) (according to Lipofectamine 2000 Protocol from Invitrogen). The transfection assay is incubated for 5 h in a cell incubator. The cells are then trypsinated and counted. The transfected cells are adjusted with fresh medium to 3×10e5 cells/ml and 40 μl of the cell suspension per well is seeded onto a white 384 MTP (Greiner) which is already charged with 10 μl/well of a test substance solution (test substances in medium without pen/strep). HEK293T cells of a logarithmically growing culture are adjusted to a concentration of 3.5×10e5 cells/ml with medium and 40 μl per well of this cell suspension are distributed onto a white 384 MTP and incubated overnight in a cell culture incubator.

Day 3: 24 h after seeding the transfected cells onto the substance plate 10 μl of supernatant are taken from each well with which the cells seeded the previous day are infected. The infected cells are incubated overnight in the cell incubator. The luciferase activity of the transfected cells on the substance plate is measured in a luminometer after the addition of 20 μl of luciferase/triton buffer.

Day 4: The luciferase activity of the infected cells is measured in a luminometer after the addition of 20 μl of luciferase/triton buffer.

The CC₅₀ value of a test substance is derived from the luciferase activity of the treated transfected cells in comparison to the untreated control cells.

The EC₅₀ value of a test substance is derived from the luciferase activity of the infected cells in comparison to the non-infected control cells.

HIV Infection in Cell Culture

The HIV test is carried out with modifications according to the method of Pauswels et al. [cf. Journal of Virological Methods 1988, 20, 309-321].

Primary human blood lymphocytes (PBLs) are enriched via Ficoll-Hypaque and stimulated in RPMI 1640 medium (from Gibco, Invitrogen Corporation, Karlsruhe, Germany), 20% fetal calf serum with phythaemagglutinin (90 μg/ml) and interleukin-2 (40 U/ml). For the infection with infectious HIV the PBLs are pelleted and the cell pellet is subsequently suspended in 1 ml of a suitably diluted HIV virus adsorption solution and incubated for one hour at 37° C. (pellet infection). Non-adsorbed virus is subsequently removed by centrifugation and the infected cells are transferred into test plates (e.g., 96 well microtiter plates) which contain the test substances in a suitable dilution.

Alternatively e.g., HIV susceptible, permanent H9 cells (ATCC or NIAIAD, USA) are used in place of normal human blood lymphocytes to test the antiviral effects of the compounds of the invention. Infected H9 cells are cultured in RPMI 1640 medium, 2% and/or 20% fetal calf serum for test purposes.

The virus adsorption solution is centrifuged and the infected cell pellet is taken up in growth medium so that it is adjusted to 1×10⁵ cells per ml. The cells infected in such a way are pipetted into the wells of 96 well microtiter plates at about 1×10⁴ cells/well (pellet infection). Alternatively the HIV is pipetted in separately after the preparation of the substance dilution in the microtiter plates and after the addition of the cells (supernatant infection).

The first vertical row of the microtiter plate contains only growth medium and cells that are not infected but are otherwise treated exactly as described above (cell control). The second vertical row of the microtiter plate contains only HIV infected cells in growth medium (virus control). The remaining wells contain the compounds of the invention in different concentrations, starting from wells of the 3rd vertical row of the microtiter plate from which on the test substances are diluted 2¹⁰ times in double steps.

Alternatively supernatant infections are carried out (see above) in which the cells are seeded into 96 well plates. The HIV virus is then added in a volume of 50 μl.

The test assays are incubated at 37° C. until the formation syncitia typical for HIV appears in the untreated virus control (between day 3 and 6 after infection), which are then evaluated either microscopically or by the p24 ELISA detection method (Vironostika, BioMerieux, The Netherlands) or photometrically or fluorometrically by Alamar Blue indicator dye. Under these test conditions about 20-100 syncitia result in the untreated virus control, whereas no syncitia appear in the untreated cell control. Correspondingly the ELISA Test shows values smaller than 0.1 for the cell controls and values between 0.1 and 2.9 for the virus controls. The photometric evaluation of the Alamar Blue treated cells shows extinctions smaller than 0.1 for the cells controls, whereas the virus controls have values between 0.1 and 3 at corresponding wavelengths.

The IC₅₀ values are determined as the concentration of the treated and infected cells at which 50% (about 20-100 syncitia) of the virus-induced syncitia are suppressed by the treatment with the compounds of the invention. The cut-off values are correspondingly set in the ELISA test and in the photometric or fluorometric determination with Alamar Blue. In addition to the determination of the antiviral effects the treated cell cultures are also investigated microscopically with respect to cytotoxic, cytostatic or cytological changes as well as with respect to solubility. Active compounds that show cell-changing, cytotoxic effects in the concentration range of activity are not assessed for their antiviral activity.

It is found that the compounds of the invention protect HIV-infected cells from virus-induced cell destruction.

In Vivo Assay

The antiviral activity of a substance, that is the ability to reduce the titer of the human immunodeficiency virus (HIV), is tested in the murine HIV model.

Human cells are infected with HIV in vitro. After the incubation the infected cells are transferred onto a collagen sponge (Gelfoam®) and transplanted subcutaneously onto the backs of immunodeficient mice. At least three groups each of 5-10 animals are used in the in vivo assay. One group represents the negative control group (placebo). One group is treated with a known antivirally active substance (e.g. Sustiva) and serves as positive control group. In further groups the substance with unknown activity is tested. For each additional test assay a group each of 5-10 animals is included. The animals are treated in different ways (e.g., orally twice daily) for a few days (e.g., 4 days). The animals are subsequently sacrificed. Blood and tissue samples can be taken for further analysis (e.g., pharmacokinetics). The collagen sponge is removed and enzymatically digested so that the cells remain. The RNA and DNA is isolated from these cells and the viral load determined, for example, by quantitative PCR.

The antiviral activity of a substance is determined relative to the activity in the placebo and positive control with the assistance of statistical methods.

### C) EXEMPLARY EMBODIMENTS FOR PHARMACEUTICAL COMPOSITIONS

The compounds of the invention can be converted into pharmaceutical preparations as follows:

Tablets:

Composition:

100 mg of the compound of example 1, 50 mg of lactose (monohydrate), 50 mg of corn starch (native), 10 mg of polyvinylpyrrolidone (PVP 25) (BASF, Ludwigshafen, Germany) and 2 mg of magnesium stearate. Tablet weight 212 mg. Diameter 8 mm, radius of curvature 12 mm.

Preparation:

The mixture of the compound of the invention, lactose and starch is granulated with a 5% solution (m/m) of the PVP in water. After drying, the granules are mixed with the magnesium stearate for 5 minutes. This mixture is compressed in a conventional tablet press (tablet format see above). A pressure of 15 kN is used as guideline for the compression.

Solution which can be Administered Orally:

Composition

500 mg of the compound from example 1, 2.5 g of polysorbate and 97 g of polyethyleneglycol 400. A single dose of 100 mg of the compound of the invention corresponds to 20 g of oral solution.

Preparation

The compound of the invention is suspended in the mixture of polyethyleneglycol and polysorbate with stirring. The stirring procedure is continued until the dissolution of the compound of the invention is complete.

I.V. Solution:

The compound of the invention is dissolved in a concentration below saturation in a physiologically acceptable solvent (e.g., isoton. saline, glucose solution 5%, PEG 400 solution 30%). The solution is sterilized by filtration and dispersed into sterile and pyrogen-free injection containers.

1. Compound of formula 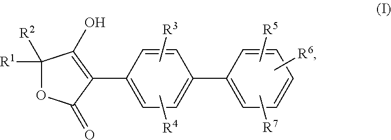
in which
R¹ and R² together with the carbon atom to which they are bonded form a group of formula 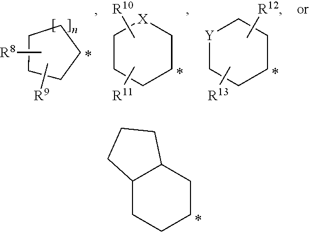
whereby
* represents the carbon atom to which R¹ and R² are bonded,
n represents the number 1, 2 or 3,
X represents an oxygen atom, a sulfur atom or NR¹⁴,
whereby
R¹⁴ represents C₁-C₆-alkyl, C₂-C₄-alkenyl, C₁-C₄-alkylsulfonyl, benzylsulfonyl, —(CH₂)ₒCOR¹⁶, —(CH₂)ₚCONR¹⁷R¹⁸, —(CH₂)_{q}NR²⁴COR²⁵ or —(CH₂)ᵥNR²⁶SO₂R²⁷,
whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄— alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²²,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
wherein phenyl, heterocyclyl and heteroaryl for their part can be substituted with 1 to 3 substituents whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
R²² represents C₃-C₇-cycloalkyl, phenyl, 5- to 10-membered heterocyclyl or 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
o represents a number 0, 1, 2 or 3,
p represents a number 0, 1, 2 or 3,
q represents a number 2 or 3,
v represents a number 2 or 3,
R¹⁶ represents C₁-C₆-alkyl, C₂-C₄-alkenyl, C₁-C₆-alkoxy, phenyl, benzyloxy or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R¹⁷ represents hydrogen, C₁-C₄-alkyl or phenyl,
whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R¹⁸ represents hydrogen or C₁-C₄-alkyl,
R²⁴ represents hydrogen or C₁-C₄-alkyl,
R²⁵ represents C₁-C₆-alkyl, C₂-C₄-alkenyl, C₁-C₆-alkoxy, phenyl, benzyloxy or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²⁶ represents hydrogen or C₁-C₄-alkyl,
R²⁷ represents C₁-C₆-alkyl, C₂-C₄-alkenyl, phenyl or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
Y represents an oxygen atom, a sulfur atom or NR¹⁵,
whereby
R¹⁵ represents C₁-C₆-alkyl, C₂-C₄-alkenyl, C₁-C₄-alkylsulfonyl, benzylsulfonyl, —(CH₂)ᵣCOR¹⁹, —(CH₂)ₛCONR²⁰R²¹, —(CH₂)ₜNR²⁸COR²⁹ or —(CH₂)_{w}NR³⁰SO₂R³¹,
whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²³,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
R²³ represents C₃-C₇-cycloalkyl, phenyl, 5- to 10-membered heterocyclyl or 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
r represents a number 0, 1, 2 or 3,
s represents a number 0, 1, 2 or 3,
t represents a number 2 or 3,
w represents a number 2 or 3,
R¹⁹ represents C₁-C₆-alkyl, C₂-C₄-alkenyl, C₁-C₆-alkoxy, phenyl, benzyloxy or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²⁰ represents hydrogen, C₁-C₄-alkyl or phenyl,
whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²¹ represents hydrogen or C₁-C₄-alkyl,
R²⁸ represents hydrogen or C₁-C₄-alkyl,
R²⁹ represents C₁-C₆-alkyl, C₂-C₄-alkenyl, C₁-C₆-alkoxy, phenyl, benzyloxy or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl und 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R³⁰ represents hydrogen or C₁-C₄-alkyl,
R³¹ represents C₁-C₆-alkyl, C₂-C₄-alkenyl, phenyl or 5- to 10-membered heterocyclyl,
whereby alkyl and alkenyl can be substituted with a substituent whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R⁸ represents hydrogen, oxo, trifluoromethyl, trifluoromethoxy, C₁-C₄-alkyl, C₁-C₄-alkoxy or C₁-C₄-alkylthio,
R⁹ represents hydrogen, C₁-C₄-alkyl or C₁-C₄-alkoxy,
R¹⁰ represents hydrogen or C₁-C₄-alkyl,
R¹¹ represents hydrogen or C₁-C₄-alkyl,
R¹² represents hydrogen or C₁-C₄-alkyl,
R¹³ represents hydrogen or C₁-C₄-alkyl,
R³ represents hydrogen, halogen, cyano, methyl, ethyl, methoxy, ethoxy or phenoxy
R⁴ represents hydrogen, halogen, methyl, ethyl, methoxy or ethoxy,
R⁵ represents hydrogen, halogen, cyano, nitro, hydroxy, amino, trifluoromethyl, trifluoromethoxy, hydroxycarbonyl, aminocarbonyl, hydroxymethyl, aminomethyl, C₁-C₄-alkyl, C₁-C₄-alkoxy, C₁-C₄-alkylamino, C₁-C₄-alkylthio, C₁-C₄-alkylcarbonyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonyl, C₁-C₄-alkylsulfonylamino, C₂-C₄-alkenylsulfonylamino, C₁-C₄-alkylsulfonyl(C₁-C₄-alkyl)amino, benzylsulfonylamino, 5- or 6-membered heteroarylsulfonylamino or 5- to 7-membered heterocyclyl,
whereby alkylaminocarbonyl, alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino,
R⁶ represents hydrogen, halogen, C₁-C₄-alkyl or C₁-C₄-alkoxy,
R⁷ represents hydrogen, halogen, C₁-C₄-alkyl or C₁-C₄-alkoxy,
or
R⁵ and R⁶ are bonded to neighboring carbon atoms, and together with the carbon atoms to which they are bonded form a 1,3-dioxolane,
or one of its salts, its solvates or the solvates of its salts,
for the treatment and/or prophylaxis of diseases.

2. Compound according to claim 1, characterized in that
R¹ and R² together with the carbon atom to which they are bonded form a group of formula 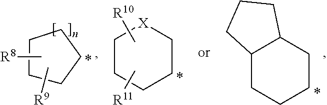
whereby
* represents the carbon atom to which R¹ and R² are bonded,
n represents the number 2,
X represents NR¹⁴,
whereby
R¹⁴ represents C₁-C₄-alkyl, C₂-C₄-alkenyl, benzylsulfonyl, —(CH₂)ₒCOR¹⁶ or —(CH₂)ₚCONR¹⁷R¹⁸,
whereby alkyl and alkenyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₃-C₇-cycloalkyl, phenyl, 5- to 10-membered heterocyclyl and 5- to 10-membered heteroaryl,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
whereby
o represents a number 1 or 2,
p represents a number 1 or 2,
R¹⁶ represents C₁-C₄-alkyl, C₁-C₄-alkoxy, phenyl or benzyloxy,
R¹⁷ represents hydrogen, C₁-C₄-alkyl or phenyl,
whereby alkyl can be substituted with a substituent, whereby the substituent is selected from the group consisting of methoxy, phenyl and 5- or 6-membered heteroaryl,
wherein phenyl for its part can be substituted with 1 to 3 substituents, whereby the substituents are selected independently of one another from the group consisting of halogen, cyano, trifluoromethyl, trifluoromethoxy and C₁-C₄-alkyl,
R¹⁸ represents hydrogen,
R⁸ represents hydrogen, C₁-C₄-alkyl or C₁-C₄-alkoxy,
R⁹ represents hydrogen or C₁-C₄-alkyl,
R¹⁰ represents hydrogen,
R¹¹ represents hydrogen,
R³ represents hydrogen, halogen, methyl, ethoxy or phenoxy,
R⁴ represents hydrogen, halogen or methyl,
R⁵ represents hydrogen, halogen, cyano, hydroxy, trifluoromethyl, trifluoromethoxy, hydroxycarbonyl, aminocarbonyl, hydroxymethyl, C₁-C₄-alkyl, C₁-C₄-alkoxy, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonyl, C₁-C₄-alkylsulfonylamino, C₂-C₄-alkenylsulfonylamino, C₁-C₄-alkylsulfonyl(C₁-C₄-alkyl)amino, benzylsulfonylamino or 5- or 6-membered heteroarylsulfonylamino,
whereby alkylaminocarbonyl, alkylcarbonylamino and alkylsulfonylamino can be substituted with a substituent, whereby the substituent is selected from the group consisting of amino, C₁-C₄-alkylamino, morpholinyl and pyrrolidinyl,
R⁶ represents hydrogen, halogen, C₁-C₄-alkyl or C₁-C₄-alkoxy,
R⁷ represents hydrogen,
or one of its salts, its solvates or the solvates of its salts,
for the treatment and/or prophylaxis of diseases.

3. Compound of formula 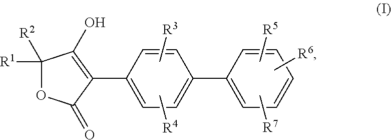
in which
R¹ and R² together with the carbon atom to which they are bonded form a group of formula 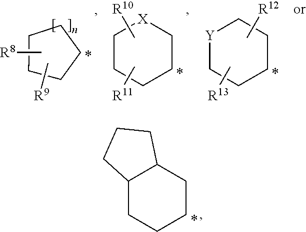
whereby
* represents the carbon atom to which R¹ and R² are bonded,
n represents the number 1, 2 or 3,
X represents an oxygen atom, a sulfur atom, or NR¹⁴,
whereby
R¹⁴ represents C₁-C₆-alkyl, C₂-C₄-alkenyl, C₁-C₄-alkylsulfonyl, benzylsulfonyl, —(CH₂)ₒCOR¹⁶, —(CH₂)ₚCONR¹⁷R¹⁸, —(CH₂)_{q}NR²⁴COR²⁵ or —(CH₂)ᵥNR²⁶SO₂R²⁷,
whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²²,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
R²² represents C₃-C₇-cycloalkyl, phenyl, 5- to 10-membered heterocyclyl or 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
o represents a number 0, 1, 2 or 3,
p represents a number 0, 1, 2 or 3,
q represents a number 2 or 3,
v represents a number 2 or 3,
R¹⁶ represents C₁-C₆-alkyl, C₂-C₄-alkenyl, C₁-C₆-alkoxy, phenyl, benzyloxy or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R¹⁷ represents hydrogen, C₁-C₄-alkyl or phenyl,
whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R¹⁸ represents hydrogen or C₁-C₄-alkyl,
R²⁴ represents hydrogen or C₁-C₄-alkyl,
R²⁵ represents C₁-C₆-alkyl, C₂-C₄-alkenyl, C₁-C₆-alkoxy, phenyl, benzyloxy or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²⁶ represents hydrogen or C₁-C₄-alkyl,
R²⁷ represents C₁-C₆-alkyl, C₂-C₄-alkenyl, phenyl or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
Y represents an oxygen atom, a sulfur atom or NR¹⁵,
whereby
R¹⁵ represents C₁-C₆-alkyl, C₂-C₄-alkenyl, C₁-C₄-alkylsulfonyl, benzylsulfonyl, —(CH₂)ᵣCOR¹⁹, —(CH₂)ₛCONR²⁰R²¹, —(CH₂)ₜNR²⁸COR²⁹ or —(CH₂)_{w}NR³⁰SO₂R³¹,
whereby alkyl, alkenyl and alkylsulfonyl can be substituted with 1 to 2 substituents, whereby the substituents are selected independently of one another from the group consisting of halogen, cyano, hydroxy, trifluoromethyl, hydroxycarbonyl, aminosulfonyl, C₁-C₄-alkoxy, C₁-C₄-alkoxycarbonyl, C₁-C₄-alkylaminocarbonyl, C₁-C₄-alkylaminosulfonyl, benzylaminosulfonyl, C₃-C₇-cycloalkyl, phenyl, 5- to 10-membered heterocyclyl, 5- to 10-membered heteroaryl and —OR²³,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl, and
wherein alkoxy can be substituted with a substituent selected from the group consisting of halogen, cyano, trifluoromethyl, hydroxy, hydroxycarbonyl, aminocarbonyl, aminosulfonyl, C₁-C₄-alkoxy, C₁-C₄-alkoxycarbonyl, C₁-C₄-alkylaminocarbonyl, C₁-C₄-alkylaminosulfonyl, C₃-C₇-cycloalkyl, phenyl, 5- to 10-membered heterocyclyl and 5- to 10-membered heteroaryl,
wherein phenyl, heterocyclyl and heteroaryl for their part can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
R²³ represents C₃-C₇-cycloalkyl, phenyl, 5- to 10-membered heterocyclyl or 5- to 10-membered heteroaryl,
wherein phenyl, heterocyclyl and heteroaryl for their part can be substituted with 1 to 3 substituents whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
r represents a number 0, 1, 2 or 3,
s represents a number 0, 1, 2 or 3,
t represents a number 2 or 3,
w represents a number 2 or 3,
R¹⁹ represents C₁-C₆-alkyl, C₂-C₄-alkenyl, C₁-C₆-alkoxy, phenyl, benzyloxy to 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²⁰ represents hydrogen, C₁-C₄-alkyl or phenyl,
whereby alkyl can be substituted with a substituent, whereby the substituent is selected from the group consisting of methoxy, C₃-C₇-cycloalkyl, phenyl, 5- to 10-membered heterocyclyl and 5- or 6-membered heteroaryl,
wherein phenyl, heterocyclyl and heteroaryl for their part can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R²¹ represents hydrogen or C₁-C₄-alkyl,
R²⁸ represents hydrogen or C₁-C₄-alkyl,
R²⁹ represents C₁-C₆-alkyl, C₂-C₄-alkenyl, C₁-C₆-alkoxy, phenyl, benzyloxy or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R³⁰ represents hydrogen or C₁-C₄-alkyl,
R³¹ represents C₁-C₆-alkyl, C₂-C₄-alkenyl, phenyl or 5- to 10-membered heterocyclyl,
whereby alkyl and alkenyl can be substituted with a substituent, whereby the substituent is selected from the group consisting of halogen, cyano, hydroxy, trifluoromethyl, hydroxycarbonyl, aminosulfonyl, C₁-C₄-alkoxycarbonyl, C₃-C₇-cycloalkyl, phenyl, phenoxy, 5- to 10-membered heterocyclyl, 5- to 10-membered heteroaryl and 5- or 6-membered heteroarylcarbonyl,
wherein phenyl, phenoxy and heteroaryl can be substituted with 1 to 3 substituents, whereby the substituents are selected independently of one another from the group consisting of halogen, cyano, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
R⁸ represents hydrogen, oxo, trifluoromethyl, trifluoromethoxy, C₁-C₄-alkyl, C₁-C₄-alkoxy or C₁-C₄-alkylthio,
R⁹ represents hydrogen, C₁-C₄-alkyl or C₁-C₄-alkoxy,
R¹⁰ represents hydrogen or C₁-C₄-alkyl,
R¹¹ represents hydrogen or C₁-C₄-alkyl,
R¹² represents hydrogen or C₁-C₄-alkyl,
R¹³ represents hydrogen or C₁-C₄-alkyl,
R³ represents hydrogen, halogen, cyano, methyl, ethyl, methoxy, ethoxy or phenoxy,
R⁴ represents hydrogen, halogen, methyl, ethyl, methoxy or ethoxy,
R⁵ represents hydroxy, amino, trifluoromethyl, trifluoromethoxy, hydroxycarbonyl, aminocarbonyl, hydroxymethyl, aminomethyl, C₁-C₄-alkylamino, C₁-C₄-alkylcarbonyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonylamino, C₂-C₄-alkenylsulfonylamino, C₁-C₄-alkylsulfonyl(C₁-C₄-alkyl)amino, benzylsulfonylamino, 5- or 6-membered heteroarylsulfonylamino or 5- to 7-membered heterocyclyl,
whereby alkylaminocarbonyl, alkylcarbonylamino and alkylsulfonylamino can be substituted with a substituent, whereby the substituent is selected from the group consisting of cyano, hydroxy, amino, hydroxycarbonyl, C₁-C₄-alkoxy, C₁-C₄-alkylamino, morpholinyl, piperidinyl, pyrrolidinyl and benzylamino,
R⁶ represents hydrogen, halogen, C₁-C₄-alkyl or C₁-C₄-alkoxy,
R⁷ represents hydrogen, halogen, C₁-C₄-alkyl or C₁-C₄-alkoxy,
or
R⁵ and R⁶ are bonded to neighboring carbon atoms and together with the carbon atoms to
which they are bonded from a 1,3-dioxolane,
or one of its salts, its solvates or the solvates of its salts.

4. Compound according to claim 3, characterized in that
R¹ and R² together with the carbon atom to which they are bonded form a group of formula 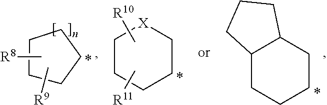
whereby
* represents the carbon atom to which R¹ and R² are bonded,
n represents the number 2,
X represents NR¹⁴,
whereby
R¹⁴ represents C₁-C₄-alkyl, C₂-C₄-alkenyl, benzylsulfonyl, —(CH₂)ₒCOR¹⁶ or —(CH₂)ₚCONR¹⁷R¹⁸,
whereby alkyl and alkenyl can be substituted with 1 to 2 substituents, whereby the substituents are selected independently of on another from the group consisting of halogen, cyano, hydroxy, trifluoromethyl, hydroxycarbonyl, aminosulfonyl, C₁-C₄-alkoxy, C₁-C₄-alkoxycarbonyl, C₃-C₇-cycloalkyl, phenyl, 5- to 10-membered heterocyclyl and 5- to 10-membered heteroaryl,
wherein phenyl, heterocyclyl and heteroaryl can be substituted with 1 to 3 substituents, whereby the substituents are selected independently of one another from the group consisting of halogen, cyano, oxo, trifluoromethyl, trifluoromethoxy, hydroxycarbonyl, hydroxysulfonyl, C₁-C₄-alkyl, C₁-C₄-alkoxy, C₁-C₄-alkylamino, C₁-C₄-alkylsulfonyl and C₁-C₄-alkoxycarbonyl,
and
whereby
o represents a number 1 or 2,
p represents a number 1 or 2,
R¹⁶ represents C₁-C₄-alkyl, C₁-C₄-alkoxy, phenyl or benzyloxy,
R¹⁷ represents hydrogen, C₁-C₄-alkyl or phenyl,
whereby alkyl can be substituted with a substituent, whereby the substituent is selected from the group consisting of methoxy, phenyl and 5- or 6-membered heteroaryl,
wherein phenyl for its part can be substituted with 1 to 3 substituents, whereby the substituents are selected independently of one another from the group consisting of halogen, cyano, trifluoromethyl, trifluoromethoxy and C₁-C₄-alkyl,
R¹⁸ represents hydrogen,
R⁸ represents hydrogen, C₁-C₄-alkyl or C₁-C₄-alkoxy,
R⁹ represents hydrogen or C₁-C₄-alkyl,
R¹⁰ represents hydrogen,
R¹¹ represents hydrogen,
R³ represents hydrogen, halogen, methyl, ethoxy or phenoxy,
R⁴ represents hydrogen, halogen or methyl,
R⁵ represents hydroxy, trifluoromethyl, trifluoromethoxy, hydroxycarbonyl, aminocarbonyl, hydroxymethyl, C₁-C₄-alkylaminocarbonyl, C₃-C₆-cycloalkylaminocarbonyl, C₁-C₄-alkylcarbonylamino, C₁-C₄-alkoxycarbonylamino, C₁-C₄-alkylsulfonylamino, C₂-C₄-alkenylsulfonylamino, C₁-C₄-alkylsulfonyl(C₁-C₄-alkyl)amino, benzylsulfonylamino or 5- or 6-membered heteroarylsulfonylamino,
whereby alkylaminocarbonyl, alkylcarbonylamino and alkylsulfonylamino can be substituted with a substituent, whereby the substituent is selected from the group consisting of amino, C₁-C₄-alkylamino, morpholinyl and pyrrolidinyl,
R⁶ represents hydrogen, halogen, C₁-C₄-alkyl or C₁-C₄-alkoxy,
R⁷ represents hydrogen,
or one of its salts, its solvates or the solvates of its salts.

5. Method for the preparation of a compound of formula (I) according to claim 1, characterized in that according to method
[A] a compound of formula 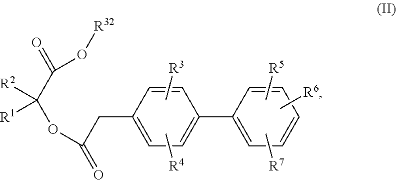
in which
R¹, R², R³, R⁴, R⁵, R⁶ and R⁷ have the meaning indicated in claim 1, and
R³² represents methyl or ethyl,
is reacted with a base,
or
[B] a compound of formula 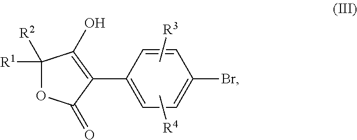
in which
R¹, R², R³ and R⁴ have the meaning indicated in claim 1,
is reacted under Suzuki coupling conditions with a compound of formula 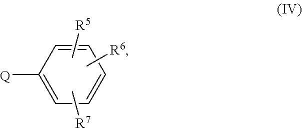
in which
R⁵, R⁶ and R⁷ have the meaning indicated in claim 1, and
Q represents —B(OH)₂, a boronic acid ester, preferably boronic acid pinacol ester, or —BF₃⁻K⁺.

6. Compound according to one of claims 3 or 4 for the treatment and/or prophylaxis of diseases.

7. Medicament comprising at least one compound according to one of claims 3 or 4 in combination with at least one inert, non-toxic, pharmaceutically acceptable excipient.

8. Use of a compound according to one of claims 3 or 4 for the manufacture of a medicament for the treatment and/or prophylaxis of diseases.

9. Medicament according to claim 7 for the treatment and/or prophylaxis of viral diseases.

10. Medicament according to claim 8 for the treatment and/or prophylaxis of HIV infections.

11. Method for controlling viral diseases in humans and animals by administration of an antivirally active amount of at least one compound according to one of claims 3 or 4, a medicament according to claim 7 or 9 or a medicament obtained according to claim 8.
